(19) United States
(12) Patent Application Publication
(10) Pub. No.: US 2008/0207554 A1
(43) Pub. Date: Aug. 28, 2008

(54) 2-5A Analogs and their Methods of Use

(75) Inventors: Leonid Beigelman, San Mateo, CA (US); Lawrence M. Blatt, San Francisco, CA (US); Harri Lonnberg, Turku (FI)

Correspondence Address: KNOBBE MARTENS OLSON & BEAR LLP, 2040 MAIN STREET, FOURTEENTH FLOOR, IRVINE, CA, 92614, US

(73) Assignee: Alios BioPharma, Inc., South San Francisco, CA (US)

(21) Appl. No.: 12/022,866

(22) Filed: Jan. 30, 2008

Related U.S. Application Data
(60) Provisional application No. 60/887,583, filed on Jan. 31, 2007.

(51) Int. Cl. A61K 31/7052; C07F 9/09; A61K 31/675; A61P 31/04; A61P 31/12; A61P 35/00; C07H 21/02

(52) U.S. Cl. 514/44; 544/244; 514/81; 544/243; 514/86; 536/25.6

(57) ABSTRACT

This invention relates to the fields of organic chemistry, pharmaceutical chemistry, biochemistry, molecular biology and medicine. In particular it relates to compounds that activate RNaseL, and to the use of the compounds for treating and/or ameliorating a disease or a condition, such as a viral infection.

This application claims priority to U.S. Provisional Application No. 60/887,583, entitled “2-5A ANALOGS AND THEIR METHODS OF USE,” filed on Jan. 31, 2007; which is incorporated herein by reference in its entirety, including any drawings

### BACKGROUND OF THE INVENTION

1. Field of the Invention

This invention relates to the fields of organic chemistry, pharmaceutical chemistry, biochemistry, molecular biology and medicine. In particular it relates to compounds that activate RNaseL, and to the use of the compounds for treating and/or ameliorating a disease or a condition, such as a viral infection.

2. Description of the Related Art

The interferon pathway is induced in mammalian cells in response to various stimuli, including viral infection. It is believed that this pathway induces the transcription of at least 200 molecules and cytokines, (immuno-regulatory substances that are secreted by cells of the immune system) involved in the defense against viral infections. These molecules and cytokines play a role in the control of cell proliferation, cell differentiation, and modulation of the immune responses.

The 2-5A system is one of the major pathways induced by the interferon pathway and has been implicated in some of its antiviral activities. This system has been described as comprising of three enzymatic activities, including 2-5A-synthetases, 2-5A-phosphodiesterase, and RNase L. 2-5A-synthetases are a family of four interferon-inducible enzymes which, upon activation by double-stranded RNA, convert ATP into the unusual series of oligomers known as 2-5A. The 2-5A-phosphodiesterase is believed to be involved in the catabolism of 2-5A from the longer oligomer. The 2-5A-dependent endoribonuclease L or RNase L is the effector enzyme of this system. RNaseL is normally inactive within the cell, so that it cannot damage the large amount of native RNA essential for normal cell function. Its activation by subnanomolar levels of 2-5A leads to the destruction of viral mRNA within the cell, and at the same time triggers the removal of the infected cell by inducing apoptosis (programmed cell death).

### SUMMARY OF THE INVENTION

Some embodiments disclosed herein relate to a compound of Formula (I) or a pharmaceutically acceptable salt, prodrug or prodrug ester thereof:

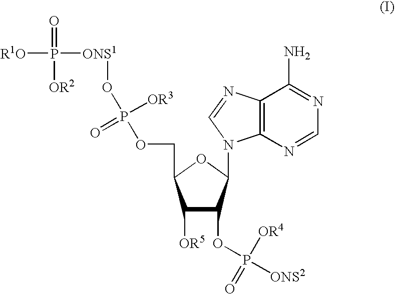

Other embodiments disclosed herein relate to a compound of Formula (Ia) or a pharmaceutically acceptable salt, prodrug or prodrug ester thereof:

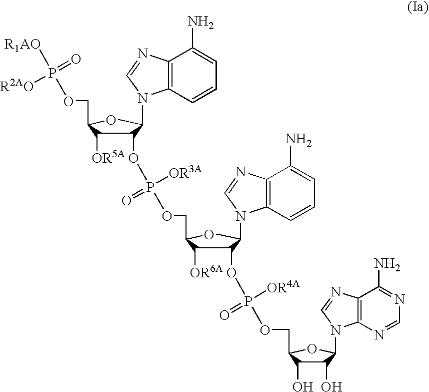

Some embodiments disclosed herein relate to methods of synthesizing a compound of Formula (I). Other embodiments disclosed herein relate to methods of synthesizing a compound of Formula (Ia).

Some embodiments disclosed herein relate to pharmaceutical compositions that can include one or more compounds of Formulae (I) and/or (Ia), and a pharmaceutically acceptable carrier, diluent, excipient or combination thereof.

Some embodiments disclosed herein relate to methods of ameliorating or treating a neoplastic disease that can include administering to a subject suffering from a neoplastic disease a therapeutically effective amount of one or more compound of Formulae (I) and/or (Ia) or a pharmaceutical composition that includes one or more compounds of Formulae (I) and/or (Ia).

Other embodiments disclosed herein relate to methods of inhibiting the growth of a tumor that can include administering to a subject having a tumor a therapeutically effective amount of one or more compound of Formulae (I) and/or (Ia) or a pharmaceutical composition that includes one or more compounds of Formulae (I) and/or (Ia).

Still other embodiments disclosed herein relate to methods of ameliorating or treating a viral infection that can include administering to a subject suffering from a viral infection a therapeutically effective amount of one or more compound of Formulae (I) and/or (Ia) or a pharmaceutical composition that includes one or more compounds of Formulae (I) and/or (Ia).

Yet still other embodiments disclosed herein relate to methods of ameliorating or treating a parasitic disease that can include administering to a subject suffering from a parasitic disease a therapeutically effective amount of one or more compound of Formulae (I) and/or (Ia) or a pharmaceutical composition that includes one or more compounds of Formulae (I) and/or (Ia).

### BRIEF DESCRIPTION OF THE DRAWINGS

FIG. 1 illustrates one method for synthesizing two exemplary phosphytiliating reagents, compounds 5 and 6.

FIG. 2 illustrates a method for synthesizing compound 15, an example of a 3-acyl building block.

FIG. 3 illustrates a method for synthesizing compound 25 and compound 26, an exemplary 3′-O-acyloxymethyl building block and an exemplary 2′-terminal building block, respectively.

FIG. 4 illustrates a method for synthesizing compound 31, an example of a 3′O-acyl protected trimer.

FIG. 5 illustrates a method for synthesizing compound 36, an exemplary 3′O-acyloxymethyl protected trimer.

FIG. 6 illustrates additional exemplary starting modified nucleosides.

FIG. 7 shows a plot of a 3′O-acyloxymethyl protected mono-nucleoside after 5 days of exposure to porcine liver esterase (PLE) in HEPES buffer.

FIG. 8 shows a plot of a 3′O-acyloxymethyl protected mono-nucleoside after 10 minutes in cell extract diluted with HEPES buffer.

FIG. 9 shows a plot of a 3′O-acyloxymethyl and phosphate protected dimer at time zero in cell extract diluted with HEPES buffer (1:10 cell extract:total volume).

FIG. 10 shows a plot of a 3′O-acyloxymethyl and phosphate protected dimer at 20 minutes in cell extract diluted with HEPES buffer (1:10 cell extract:total volume).

FIG. 11 shows plots of a 3′O-acyloxymethyl and phosphate protected dimer at 1 hour and 20 minutes and at 3 hours and 40 minutes in cell extract diluted with HEPES buffer (1:10 cell extract:total volume).

FIG. 12 shows plots of a 3′O-acyloxymethyl and phosphate protected dimer in cell at 22 hours and at 2 days in cell extract diluted with HEPES buffer (1:10 cell extract:total volume).

FIG. 13 shows a plot of a 3′O-acyloxymethyl and phosphate protected dimer at 7 days in cell extract diluted with HEPES buffer (1:10 cell extract:total volume).

FIG. 14 shows a plot of a 3′O-acyloxymethyl and phosphate protected dimer at 14 days in cell extract diluted with HEPES buffer (1:10 cell extract:total volume).

FIG. 15 shows a plot of a 3′O-acyloxymethyl and phosphate protected dimer at 15 days in cell extract diluted with HEPES buffer (3:10 cell extract:total volume).

FIG. 16 shows a plot of a 3′O-acyloxymethyl and phosphate protected dimer at 19 days in cell extract diluted with HEPES buffer (3:10 cell extract:total volume).

FIG. 17 shows a plot of a 3′O-acyloxymethyl and phosphate protected dimer at 28 days in cell extract diluted with HEPES buffer (3:10 cell extract:total volume)

FIG. 18 shows a plot of a 3′O-acyloxymethyl and phosphate protected dimer after 20 minutes of exposure PLE in HEPES buffer.

FIG. 19 shows a plot of a 3′O-acyloxymethyl and phosphate protected dimer after 2 hours of exposure PLE in HEPES buffer.

FIG. 20 shows a plot of a 3′O-acyloxymethyl and phosphate protected dimer after 20 hours of exposure PLE in HEPES buffer.

### DETAILED DESCRIPTION OF THE PREFERRED EMBODIMENT

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

As used herein, any “R” group(s) such as, without limitation, R¹, R^{1a} and R^{1b}, represent substituents that can be attached to the indicated atom. A non-limiting list of R groups include, but are not limited to, hydrogen, alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxy, alkoxy, aryloxy, acyl, ester, mercapto, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, and amino, including mono- and di-substituted amino groups, and the protected derivatives thereof. An R group may be substituted or unsubstituted. If two “R” groups are covalently bonded to the same atom or to adjacent atoms, then they may be “taken together” as defined herein to form a cycloalkyl, cycloalkenyl, cycloalkynyl, aryl, heteroaryl or heteroalicyclyl group. For example, without limitation, if Rₐ and R_{b} of an NRₐR_{b} group are indicated to be “taken together”, it means that they are covalently bonded to one another at their terminal atoms to form a ring that includes the nitrogen:

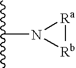

Whenever a group is described as being “optionally substituted” that group may be unsubstituted or substituted with one or more of the indicated substituents. Likewise, when a group is described as being “unsubstituted or substituted” if substituted, the substituent may be selected from one or more the indicated substituents.

The term “substituted” has its ordinary meaning, as found in numerous contemporary patents from the related art. See, for example, U.S. Pat. Nos. 6,509,331; 6,506,787; 6,500,825; 5,922,683; 5,886,210; 5,874,443; and 6,350,759; all of which are incorporated herein in their entireties by reference. Examples of suitable substituents include but are not limited to hydrogen, alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, ester, mercapto, alkylthio, arylthio, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, and amino, including mono- and di-substituted amino groups, and the protected derivatives thereof. Each of the substituents can be further substituted. The other above-listed patents also provide standard definitions for the term “substituted” that are well-understood by those of skill in the art.

As used herein, “Cₘ to Cₙ” in which “m” and “n” are integers refers to the number of carbon atoms in an alkyl, alkenyl or alkynyl group or the number of carbon atoms in the ring of a cycloalkyl, cycloalkenyl, cycloalkynyl, aryl, heteroaryl or heteroalicyclyl group. That is, the alkyl, alkenyl, alkynyl, ring of the cycloalkyl, ring of the cycloalkenyl, ring of the cycloalkynyl, ring of the aryl, ring of the heteroaryl or ring of the heteroalicyclyl can contain from “m” to “n”, inclusive, carbon atoms. Thus, for example, a “C₁ to C₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—. If no “m” and “n” are designated with regard to an alkyl, alkenyl, alkynyl, cycloalkyl cycloalkenyl, cycloalkynyl, aryl, heteroaryl or heteroalicyclyl group, the broadest range described in these definitions is to be assumed.

As used herein, “alkyl” refers to a straight or branched hydrocarbon chain that comprises a fully saturated (no double or triple bonds) hydrocarbon group. The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 10 carbon atoms. The alkyl group could also be a lower alkyl having 1 to 5 carbon atoms. The alkyl group of the compounds may be designated as “C₁-C₄ alkyl” or similar designations. By way of example only, “C₁-C₄ alkyl” indicates that there are one to four carbon atoms in the alkyl chain, i.e., the alkyl chain is selected from methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like.

The alkyl group may be substituted or unsubstituted. When substituted, the substituent group(s) is(are) one or more group(s) individually and independently selected from alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, ester, mercapto, alkylthio, arylthio, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, and amino, including mono- and di-substituted amino groups, and the protected derivatives thereof. Wherever a substituent is described as being “optionally substituted” that substitutent may be substituted with one of the above substituents.

As used herein, “alkenyl” refers to an alkyl group that contains in the straight or branched hydrocarbon chain one or more double bonds. An alkenyl group of this invention may be unsubstituted or substituted. When substituted, the substituent(s) may be selected from the same groups disclosed above with regard to alkyl group substitution unless otherwise indicated.

As used herein, “alkynyl” refers to an alkyl group that contains in the straight or branched hydrocarbon chain one or more triple bonds. An alkynyl group of this invention may be unsubstituted or substituted. When substituted, the substituent(s) may be selected from the same groups disclosed above with regard to alkyl group substitution unless otherwise indicated.

As used herein, “aryl” refers to a carbocyclic (all carbon) monocyclic or multicyclic aromatic ring system that has a fully delocalized pi-electron system. Examples of aryl groups include, but are not limited to, benzene, naphthalene and azulene. An aryl group of this invention may be substituted or unsubstituted. When substituted, hydrogen atoms are replaced by substituent group(s) that is(are) one or more group(s) independently selected from alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxy, alkoxy, aryloxy, acyl, ester, mercapto, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, and amino, including mono- and di-substituted amino groups, and the protected derivatives thereof, unless the substituent groups are otherwise indicated.

As used herein, “heteroaryl” refers to a monocyclic or multicyclic aromatic ring system (a ring system with fully delocalized pi-electron system) that contain(s) one or more heteroatoms, that is, an element other than carbon, including but not limited to, nitrogen, oxygen and sulfur. Examples of heteroaryl rings include, but are not limited to, furan, furazan, thiophene, benzothiophene, phthalazine, pyrrole, oxazole, benzoxazole, 1,2,3-oxadiazole, 1,2,4-oxadiazole, thiazole, 1,2,3-thiadiazole, 1,2,4-thiadiazole, benzothiazole, imidazole, benzimidazole, indole, indazole, pyrazole, benzopyrazole, isoxazole, benzoisoxazole, isothiazole, triazole, benzotriazole, thiadiazole, tetrazole, pyridine, pyridazine, pyrimidine, pyrazine, purine, pteridine, quinoline, isoquinoline, quinazoline, quinoxaline, cinnoline, and triazine. A heteroaryl group of this invention may be substituted or unsubstituted. When substituted, hydrogen atoms are replaced by substituent group(s) that is(are) one or more group(s) independently selected from alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxy, alkoxy, aryloxy, acyl, ester, mercapto, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, and amino, including mono- and di-substituted amino groups, and the protected derivatives thereof.

An “aralkyl” is an aryl group connected, as a substituent, via a lower alkylene group. The lower alkylene and aryl group of an aralkyl may be substituted or unsubstituted. Examples include but are not limited to benzyl, substituted benzyl, 2-phenylalkyl, 3-phenylalkyl, and naphtylalkyl.

A “heteroaralkyl” is heteroaryl group connected, as a substituent, via a lower alkylene group. The lower alkylene and heteroaryl group of heteroaralkyl may be substituted or unsubstituted. Examples include but are not limited to 2-thienylalkyl, 3-thienylalkyl, furylalkyl, thienylalkyl, pyrrolylalkyl, pyridylalkyl, isoxazollylalkyl, and imidazolylalkyl, and their substituted as well as benzo-fused analogs.

“Lower alkylene groups” are straight-chained tethering groups, forming bonds to connect molecular fragments via their terminal carbon atoms. Examples include but are not limited to methylene (—CH₂—), ethylene (—CH₂CH₂—), propylene (—CH₂CH₂CH₂—), and

As used herein, “cycloalkyl” refers to a completely saturated (no double bonds) mono- or multi-cyclic hydrocarbon ring system. When composed of two or more rings, the rings may be joined together in a fused, bridged or spiro-connected fashion. Cycloalkyl groups of this invention may range from C₃ to C₁₀, in other embodiments it may range from C₃ to C₈. A cycloalkyl group may be unsubstituted or substituted. Typical cycloalkyl groups include, but are in no way limited to, cyclopropyl, cyclobutyl, cyclopentyl, cyclohexyl, and the like. If substituted, the substituent(s) may be an alkyl or selected from those substituents indicated above with respect to substitution of an alkyl group unless otherwise indicated.

As used herein, “cycloalkenyl” refers to a cycloalkyl group that contains one or more double bonds in the ring although, if there is more than one, the double bonds cannot form a fully delocalized pi-electron system in the ring (otherwise the group would be “aryl,” as defined herein). When composed of two or more rings, the rings may be connected together in a fused, bridged or spiro-connected fashion. A cycloalkenyl group of this invention may be unsubstituted or substituted. When substituted, the substituent(s) may be an alkyl or selected from the substituents disclosed above with respect to alkyl group substitution unless otherwise indicated.

As used herein, “cycloalkynyl” refers to a cycloalkyl group that contains one or more triple bonds in the ring. When composed of two or more rings, the rings may be joined together in a fused, bridged or spiro-connected fashion. A cycloalkynyl group of this invention may be unsubstituted or substituted. When substituted, the substituent(s) may be an alkyl or selected from the substituents disclosed above with respect to alkyl group substitution unless otherwise indicated.

As used herein, “heteroalicyclic” or “heteroalicyclyl” refers to a stable 3- to 18 membered ring which consists of carbon atoms and from one to five heteroatoms selected from nitrogen, oxygen and sulfur. For the purpose of this invention, the “heteroalicyclic” or “heteroalicyclyl” may be monocyclic, bicyclic, tricyclic, or tetracyclic ring system, which may be joined together in a fused, bridged or spiro-connected fashion; and the nitrogen, carbon and sulfur atoms in the “heteroalicyclic” or “heteroalicyclyl” may be optionally oxidized; the nitrogen may be optionally quaternized; and the rings may also contain one or more double bonds provided that they do not form a fully delocalized pi-electron system throughout all the rings. Heteroalicyclyl groups may be unsubstituted or substituted. When substituted, the substituent(s) may be one or more groups independently selected from alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, ester, mercapto, alkylthio, arylthio, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, haloalkyl, haloalkoxy, trihalomethanesulfonyl, trihalomethanesulfonamido, and amino, including mono- and di-substituted amino groups, and the protected derivatives thereof. Examples of such “heteroalicyclic” or “heteroalicyclyl” include but are not limited to, azepinyl, acridinyl, carbazolyl, cinnolinyl, 1,3-dioxin, 1,3-dioxane, 1,4-dioxane, 1,2-dioxolanyl, 1,3-dioxolanyl, 1,4-dioxolanyl, 1,3-oxathiane, 1,4-oxathiin, 1,3-oxathiolane, 1,3-dithiole, 1,3-dithiolane, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, trioxane, hexahydro-1,3,5-triazine, imidazolinyl, imidazolidine, isoxazoline, isoxazolidine, oxazoline, oxazolidine, oxazolidinone, thiazoline, thiazolidine, morpholinyl, oxiranyl, piperidinyl N-Oxide, piperidinyl, piperazinyl, pyrrolidinyl, pyrrolidone, pyrrolidione, 4-piperidonyl, pyrazoline, pyrazolidinyl, 2-oxopyrrolidinyl, tetrahydropyran, 4H-pyran, tetrahydrothiopyran, thiamorpholinyl, thiamorpholinyl sulfoxide, thiamorpholinyl sulfone, and their benzo-fused analogs (e.g., benzimidazolidinone, tetrahydroquinoline, 3,4-methylenedioxyphenyl).

A “(heteroalicyclyl)alkyl” is a heterocyclic or a heteroalicyclylic group connected, as a substituent, via a lower alkylene group. The lower alkylene and heterocyclic or a heterocyclyl of a (heteroalicyclyl)alkyl may be substituted or unsubstituted. Examples include but are not limited tetrahydro-2H-pyran-4-yl)methyl, (piperidin-4-yl)ethyl, (piperidin-4-yl)propyl, (tetrahydro-2H-thiopyran-4-yl)methyl, and (1,3-thiazinan-4-yl)methyl.

As used herein, “alkoxy” refers to the formula —OR wherein R is an alkyl is defined as above, e.g. methoxy, ethoxy, n-propoxy, 1-methylethoxy (isopropoxy), n-butoxy, iso-butoxy, sec-butoxy, tert-butoxy, and the like. An alkoxy may be substituted or unsubstituted.

As used herein, “acyl” refers to a hydrogen, alkyl, alkenyl, alkynyl, or aryl connected, as substituents, via a carbonyl group. Examples include formyl, acetyl, propanoyl, benzoyl, and acryl. An acyl may be substituted or unsubstituted. An acyl may be substituted or unsubstituted.

As used herein, “hydroxyalkyl” refers to an alkyl group in which one or more of the hydrogen atoms are replaced by hydroxy group. Exemplary hydroxyalkyl groups include but are not limited to, 2-hydroxyethyl, 3-hydroxypropyl, 2-hydroxypropyl, and 2,2-dihydroxyethyl. A hydroxyalkyl may be substituted or unsubstituted.

As used herein, “haloalkyl” refers to an alkyl group in which one or more of the hydrogen atoms are replaced by halogen (e.g., mono-haloalkyl, di-haloalkyl and tri-haloalkyl). Such groups include but are not limited to, chloromethyl, fluoromethyl, difluoromethyl, trifluoromethyl and 1-chloro-2-fluoromethyl, 2-fluoroisobutyl. A haloalkyl may be substituted or unsubstituted.

As used herein, “haloalkoxy” refers to an alkoxy group in which one or more of the hydrogen atoms are replaced by halogen (e.g., mono-haloalkoxy, di-haloalkoxy and tri-haloalkoxy). Such groups include but are not limited to, chloromethoxy, fluoromethoxy, difluoromethoxy, trifluoromethoxy and 1-chloro-2-fluoromethoxy, 2-fluoroisobutoxy. A haloalkoxy may be substituted or unsubstituted.

As used herein, “aryloxy” and “arylthio” refers to RO— and RS—, in which R is an aryl, such as but not limited to phenyl. Both an aryloxy and arylthio may be substituted or unsubstituted.

A “sulfenyl” group refers to an “—SR” group in which R can be hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl. A sulfenyl may be substituted or unsubstituted.

A “sulfinyl” group refers to an “—S(═O)—R” group in which R can be the same as defined with respect to sulfenyl. A sulfinyl may be substituted or unsubstituted.

A “sulfonyl” group refers to an “SO₂R” group in which R can be the same as defined with respect to sulfenyl. A sulfonyl may be substituted or unsubstituted.

An “O-carboxy” group refers to a “RC(═O)O—” group in which R can be hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl, as defined herein. An O-carboxy may be substituted or unsubstituted.

A “C-carboxy” group refers to a “—C(═O)R” group in which R can be the same as defined with respect to O-carboxy. A C-carboxy may be substituted or unsubstituted.

A “thiocarbonyl” group refers to a “—C(═S)R” group in which R can be the same as defined with respect to O-carboxy. A thiocarbonyl may be substituted or unsubstituted.

A “trihalomethanesulfonyl” group refers to an “X₃CSO₂—” group wherein X is a halogen.

A “trihalomethanesulfonamido” group refers to an “X₃CS(O)₂ R_{A}N—” group wherein X is a halogen and R defined with respect to O-carboxy.

The term “amino” as used herein refers to a —NH₂ group.

As used herein, the term “hydroxy” refers to a —OH group.

A “cyano” group refers to a “—CN” group.

The term “azido” as used herein refers to a —N₃ group.

An “isocyanato” group refers to a “—NCO” group.

A “thiocyanato” group refers to a “—CNS” group.

An “isothiocyanato” group refers to an “—NCS” group.

A “mercapto” group refers to an “—SH” group.

A “carbonyl” group refers to a C═O group.

An “S-sulfonamido” group refers to a “—SO₂NR_{A}R_{B}” group in which R_{A} and R_{B} can be the same as R defined with respect to O-carboxy. An S-sulfonamido may be substituted or unsubstituted.

An “N-sulfonamido” group refers to a “RSO₂N(R_{A})—” group in which R and R_{A} can be the same as R defined with respect to O-carboxy. A sulfonyl may be substituted or unsubstituted.

A “trihalomethanesulfonamido” group refers to an “X₃CSO₂N(R)—” group with X as halogen and R can be the same as defined with respect to O-carboxy. A trihalomethanesulfonamido may be substituted or unsubstituted.

An “O-carbamyl” group refers to a “—OC(═O)NR_{A}R_{B}” group in which R_{A} and R_{B} can be the same as R defined with respect to O-carboxy. An O-carbamyl may be substituted or unsubstituted.

An “N-carbamyl” group refers to an “ROC(═O)NR_{A}—” group in which R and R_{A} can be the same as R defined with respect to O-carboxy. An N-carbamyl may be substituted or unsubstituted.

An “O-thiocarbamyl” group refers to a “—OC(═S)—NR_{A}R_{B}” group in which R_{A} and R_{B} can be the same as R defined with respect to O-carboxy. An O-thiocarbamyl may be substituted or unsubstituted.

An “N-thiocarbamyl” group refers to an “ROC(═S)NR_{A}—” group in which R and R_{A} can be the same as R defined with respect to O-carboxy. An N-thiocarbamyl may be substituted or unsubstituted.

A “C-amido” group refers to a “—C(═O)NR_{A}R_{B}” group in which R_{A} and R_{B} can be the same as R defined with respect to O-carboxy. A C-amido may be substituted or unsubstituted.

An “N-amido” group refers to a “RC(═O)NR_{A}—” group in which R and R_{A} can be the same as R defined with respect to O-carboxy. An N-amido may be substituted or unsubstituted.

An “ester” refers to a “—C(═O)OR” group in which R can be the same as defined with respect to O-carboxy. An ester may be substituted or unsubstituted.

As used herein, “alkylcarbonyl” refers to a group of the formula —C(═O)Rₐ wherein Rₐ can be an alkyl, such as a C₁₋₄ alkyl, as defined herein. An alkylcarbonyl can be substituted or unsubstituted.

The term “alkoxycarbonyl” as used herein refers to a group of the formula —C(═O)ORₐ wherein Rₐ can be the same as defined with respect to alkylcarbonyl. An alkoxycarbonyl can be substituted or unsubstituted.

As used herein, “alkylaminocarbonyl” refers to a group of the formula —C(═O)NH_{R}, wherein Rₐ can be an alkyl, such as a C₁₋₄ alkyl, as defined herein. An alkylaminocarbonyl can be substituted or unsubstituted.

As used herein, the term “levulinoyl” refers to a —C(═O)CH₂CH₂C(═O)CH₃ group.

The term “halogen atom,” as used herein, means any one of the radio-stable atoms of column 7 of the Periodic Table of the Elements, i.e., fluorine, chlorine, bromine, or iodine, with bromine and chlorine being preferred.

Where the numbers of substituents is not specified (e.g. haloalkyl), there may be one or more substituents present. For example “haloalkyl” may include one or more of the same or different halogens. As another example, “C₁-C₃ alkoxyphenyl” may include one or more of the same or different alkoxy groups containing one, two or three atoms.

As used herein, the abbreviations for any protective groups, amino acids and other compounds, are, unless indicated otherwise, in accord with their common usage, recognized abbreviations, or the IUPAC-IUB Commission on Biochemical Nomenclature (See, Biochem. 11:942-944 (1972)).

As used herein, the term “nucleoside” refers to a compound composed of any pentose or modified pentose moiety attached to a specific portion of a heterocyclic base or derivative thereof such as the 9-position of a purine, 1-position of a pyrimidine, or an equivalent position of a heterocyclic base derivative. In some instances, the nucleoside can be a nucleoside drug analog. As used herein, the term “nucleoside drug analog” refers to a compound composed of a nucleoside that has therapeutic activity (e.g., antiviral, anti-neoplastic, anti-parasitic and/or antibacterial activity).

As used herein, the term “nucleotide” refers to a phosphate ester substituted on the 5′-position of a nucleoside or an equivalent position on a derivative thereof.

As used herein, the terms “protected nucleoside” and “protected nucleoside derivative” refers to a nucleoside and nucleoside derivative, respectively, in which one or more hydroxy groups attached to the ribose or deoxyribose ring are protected with one or more protecting groups. An example of protected nucleoside is an adenosine in which the oxygen at the 3′-position is protected with a protecting group such as methyl group or a levulinoyl group.

As used herein, the term “heterocyclic base” refers to a purine, a pyrimidine and derivatives thereof. The term “purine” refers to a substituted purine, its tautomers and analogs thereof. Similarly, the term “pyrimidine” refers to a substituted pyrimidine, its tautomers and analogs thereof. Exemplary purines include, but are not limited to, purine, adenine, guanine, hypoxanthine, xanthine, theobromine, caffeine, uric acid and isoguanine. Examples of pyrimidines include, but are not limited to, cytosine, thymine, uracil, and derivatives thereof. An example of an analog of a purine is 1,2,4-triazole-3-carboxamide.

As used herein, the term “protected heterocyclic base” refers to a heterocyclic base in which one or more amino groups attached to the base are protected with one or more suitable protecting groups and/or one or more —NH groups present in a ring of the heterocyclic base are protected with one or more suitable protecting groups. When more than one protecting group is present, the protecting groups can be the same or different.

The terms “derivative,” “variant,” or other similar term refers to a compound that is an analog of the other compound.

The terms “protecting group” and “protecting groups” as used herein refer to any atom or group of atoms that is added to a molecule in order to prevent existing groups in the molecule from undergoing unwanted chemical reactions. Examples of protecting group moieties are described in T. W. Greene and P. G. M. Wuts, Protective Groups in Organic Synthesis, 3. Ed. John Wiley & Sons, 1999, and in J. F. W. McOmie, Protective Groups in Organic Chemistry Plenum Press, 1973, both of which are hereby incorporated by reference. The protecting group moiety may be chosen in such a way, that they are stable to the reaction conditions applied and readily removed at a convenient stage using methodology known from the art. A non-limiting list of protecting groups include benzyl; substituted benzyl; alkylcarbonyls (e.g., t-butoxycarbonyl (BOC)); arylalkylcarbonyls (e.g., benzyloxycarbonyl, benzoyl); substituted methyl ether (e.g. methoxymethyl ether); substituted ethyl ether; a substituted benzyl ether; tetrahydropyranyl ether; silyl ethers (e.g., trimethylsilyl, triethylsilyl, triisopropylsilyl, t-butyldimethylsilyl, or t-butyldiphenylsilyl); esters (e.g. benzoate ester); carbonates (e.g. methoxymethylcarbonate); sulfonates (e.g. tosylate, mesylate); acyclic ketal (e.g. dimethyl acetal); cyclic ketals (e.g., 1,3-dioxane or 1,3-dioxolanes); acyclic acetal; cyclic acetal; acyclic hemiacetal; cyclic hemiacetal; and cyclic dithioketals (e.g., 1,3-dithiane or 1,3-dithiolane).

“Leaving group” as used herein refers to any atom or moiety that is capable of being displaced by another atom or moiety in a chemical reaction. More specifically, in some embodiments, “leaving group” refers to the atom or moiety that is displaced in a nucleophilic substitution reaction. In some embodiments, “leaving groups” are any atoms or moieties that are conjugate bases of a strong acid. Non-limiting characteristics and examples of leaving groups can be found, for example in Organic Chemistry, 2d ed., Francis Carey (1992), pages 328-331; Introduction to Organic Chemistry, 2d ed., Andrew Streitwieser and Clayton Heathcock (1981), pages 169-171; and Organic Chemistry, 5^{th} ed., John McMurry (2000), pages 398 and 408; all of which are incorporated herein by reference in their entirety.

A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. Conventional procedures for the selection and preparation of suitable prodrug derivatives are described, for example, in Design of Prodrugs, (ed. H. Bundgaard, Elsevier, 1985), which is hereby incorporated herein by reference in its entirety.

The term “pro-drug ester” refers to derivatives of the compounds disclosed herein formed by the addition of any of several ester-forming groups that are hydrolyzed under physiological conditions. Examples of pro-drug ester groups include pivaloyloxymethyl, acetoxymethyl, phthalidyl, indanyl and methoxymethyl, as well as other such groups known in the art, including a (5-R-2-oxo-1,3-dioxolen-4-yl)methyl group. Other examples of pro-drug ester groups can be found in, for example, T. Higuchi and V. Stella, in “Pro-drugs as Novel Delivery Systems”, Vol. 14, A.C.S. Symposium Series, American Chemical Society (1975); and “Bioreversible Carriers in Drug Design: Theory and Application”, edited by E. B. Roche, Pergamon Press: New York, 14-21 (1987) (providing examples of esters useful as prodrugs for compounds containing carboxyl groups). Each of the above-mentioned references is herein incorporated by reference in their entirety.

The term “pharmaceutically acceptable salt” refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), sulfuric acid, nitric acid, phosphoric acid and the like. Pharmaceutical salts can also be obtained by reacting a compound with an organic acid such as aliphatic or aromatic carboxylic or sulfonic acids, for example acetic, succinic, lactic, malic, tartaric, citric, ascorbic, nicotinic, methanesulfonic, ethanesulfonic, p-toluensulfonic, salicylic or naphthalenesulfonic acid. Pharmaceutical salts can also be obtained by reacting a compound with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, C₁-C₇ alkylamine, cyclohexylamine, triethanolamine, ethylenediamine, and salts with amino acids such as arginine, lysine, and the like.

It is understood that, in any compound described herein having one or more chiral centers, if an absolute stereochemistry is not expressly indicated, then each center may independently be of R-configuration or S-configuration or a mixture thereof. Thus, the compounds provided herein may be enatiomerically pure or be stereoisomeric mixtures. In addition it is understood that, in any compound described herein having one or more double bond(s) generating geometrical isomers that can be defined as E or Z each double bond may independently be E or Z a mixture thereof. Likewise, all tautomeric forms are also intended to be included.

### Compounds

Some embodiments disclosed herein relates to a compound of Formula (I) as shown herein, or a pharmaceutically acceptable salt, prodrug or prodrug ester in which each R¹, R², R³ and R⁴ can be each independently absent, hydrogen or

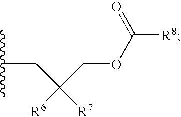

each R⁵ can be each independently selected from hydrogen, —C(═O)R⁹, and —C(R¹⁰)₂—O—C(═O)R¹¹; each R⁶ and each R⁷ can be each independently selected from —C≡N, an optionally substituted 1-oxoalkyl, an optionally substituted alkoxycarbonyl and an optionally substituted alkylaminocarbonyl; each R⁸, each R⁹, each R¹⁰ and each R¹¹ can be each hydrogen or an optionally substituted C₁₋₄-alkyl; NS¹ and NS² can be independently selected from a nucleoside, a protected nucleoside, a nucleoside derivative and a protected nucleoside derivative.

In some embodiments, R⁶ can be —C≡N. In some embodiment, R⁷ can be an optionally substituted alkoxycarbonyl. In an embodiment, the optionally substituted C₁₋₄ alkoxycarbonyl can be —C(═O)OCH₃. In other embodiments, R⁷ can be an optionally substituted alkylaminocarbonyl. In an embodiment, the optionally substituted C₁₋₄ alkylaminocarbonyl can be —C(═O)NHCH₂CH₃. In still other embodiments, R⁷ can be an optionally substituted 1-oxoalkyl. In an embodiment, the optionally substituted 1-oxoalkyl can be —C(═O)CH₃. In some embodiments, R⁸ can be an optionally substituted C₁₋₄-alkyl. Exemplary optionally substituted C₁₋₄-alkyl include methyl, ethyl, n-propyl, isopropyl, n-butyl, iso-butyl and tert-butyl.

In some embodiments, R⁵ can be —C(═O)R⁹. In an embodiment, R⁹ can be unsubstituted or substituted C₁₋₄-alkyl such as methyl, ethyl, n-propyl, isopropyl, n-butyl, iso-butyl and tert-butyl. In other embodiments, R⁵ can be —C(R¹⁰)₂—O—C(═O)R¹¹. In an embodiment, each R¹⁰ can be hydrogen. In some embodiments, R¹¹ can be unsubstituted or substituted C₁₋₄-alkyl, for example, a methyl.

Suitable,

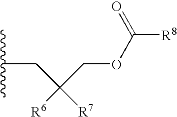

include, but are not limited to, the following:

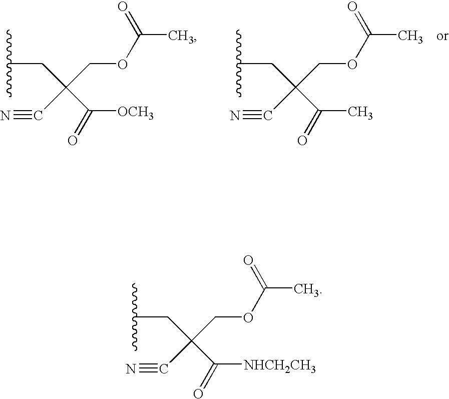

In some embodiments, NS¹ can be selected from an anti-neoplastic agent, an anti-viral agent and an anti-parasitic agent. The anti-viral agent can be activity against various viruses, including, but not limited to, one or more of the following: an adenovirus, an Alphaviridae, an Arbovirus, an Astrovirus, a Bunyaviridae, a Coronaviridae, a Filoviridae, a Flaviviridae, a Hepadnaviridae, a Herpesviridae, an Alphaherpesvirinae, a Betaherpesvirinae, a Gammaherpesvirinae, a Norwalk Virus, an Astroviridae, a Caliciviridae, an Orthomyxoviridae, a Paramyxoviridae, a Paramyxoviruses, a Rubulavirus, a Morbillivirus, a Papovaviridae, a Parvoviridae, a Picornaviridae, an Aphthoviridae, a Cardioviridae, an Enteroviridae, a Coxsackie virus, a Polio Virus, a Rhinoviridae, a Phycodnaviridae, a Poxviridae, a Reoviridae, a Rotavirus, a Retroviridae, an A-Type Retrovirus, an Immunodeficiency Virus, a Leukemia Viruses, an Avian Sarcoma Viruses, a Rhabdoviruses, a Rubiviridae and/or a Togaviridae. When NS¹ is an anti-neoplastic agent, in some embodiments, the compound of Formula (I) can have activity against cancer, tumors (e.g., solid tumors) and the like. Similarly, when NS¹ is an anti-parasitic agent, in an embodiment, the compound of Formula (I) can have activity against Chagas' disease.

An exemplary structure of NS¹ is:

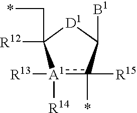

in which A¹ can be selected from C (carbon), O (oxygen) and S (sulfur); B¹ can be an optionally substituted heterocyclic base or a derivative thereof; D¹ can be C═CH₂ or O (oxygen); R¹² can be selected from hydrogen, azido, —CN, an optionally substituted C₁₋₄ alkyl and an optionally substituted C₁₋₄ alkoxy; R¹³ can be absent or selected from hydrogen halogen, hydroxy and an optionally substituted C₁₋₄ alkyl; R¹⁴ can be absent or selected from hydrogen, halogen, azido, amino, hydroxy, —OC(═O)R¹⁶, and —OC(R¹⁷)₂—O—C(═O)R¹⁸; R¹⁵ can be selected from hydrogen, halogen, hydroxy, —CN, —NC, an optionally substituted C₁₋₄ alkyl, an optionally substituted haloalkyl and an optionally substituted hydroxyalkyl; each R¹⁶, each R¹⁷ and each R¹⁸ can be independently hydrogen or an optionally substituted C₁₋₄-alkyl; and * represents a point of attachment.

In some embodiments, R¹⁴ can be —OC(═O)R¹⁶. In some embodiments, R¹⁶ can be an unsubstituted or substituted C₁₋₄ alkyl. In an embodiment, R¹⁴ can be —OC(═O)CH₃. In other embodiments, R¹⁴ can be —OC(R¹⁷)₂—O—C(═O)R¹⁸. In an embodiment, each R¹⁷ can be hydrogen. In some embodiments, R¹⁸ can be an unsubstituted or substituted C₁₋₄ alkyl. In an embodiment, R¹⁴ can be —OCH₂—O—C(═O)CH₃, —OCH₂—O—C(═O)(n-butyl) or —OCH₂—O—C(═O)(t-butyl).

In some embodiments, the heterocyclic base or derivative thereof represented by B¹ can be selected from:

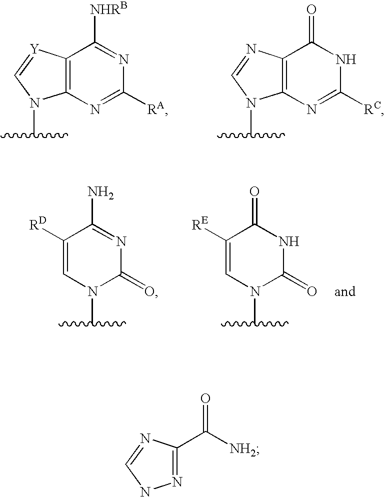

in which R^{A} can be hydrogen or halogen; R_{B} can be hydrogen, an optionally substituted C₁₋₄ alkyl, or an optionally substituted C₃₋₈ cycloalkyl; R^{C} can be hydrogen or amino; R^{D} can be hydrogen or halogen; R^{E} can be hydrogen or an optionally substituted C₁₋₄ alkyl; and Y can be N (nitrogen) or CR^{F}, wherein R^{F} hydrogen, halogen or an optionally substituted C₁₋₄ alkyl.

Examples of suitable NS¹ groups include, but are not limited to, the following:

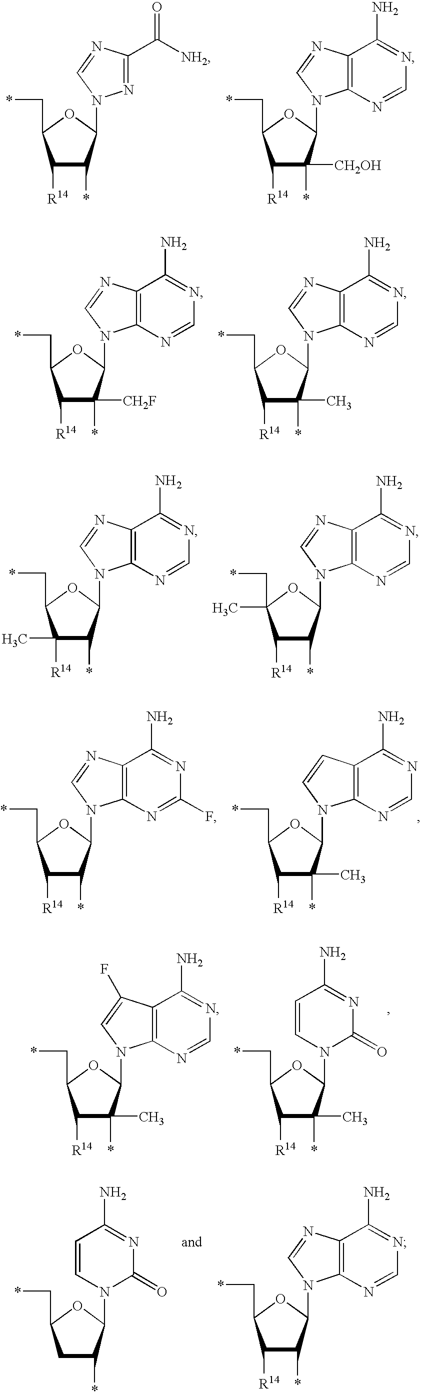

in which R¹⁴ can be absent or selected from hydrogen, halogen, azido, amino, hydroxy, —OC(═O)R¹⁶, and —OC(R¹⁷)₂—O—C(═O)R¹⁸, wherein R¹⁶, each R¹⁷ and R¹⁸ can be independently hydrogen or an optionally substituted C₁₋₄-alkyl; and * represents a point of attachment. In some embodiments, R¹⁴ can be —OC(═O)R¹⁶. In some embodiments, R¹⁶ can be an unsubstituted or substituted C₁₋₄ alkyl. In an embodiment, R¹⁴ can be —OC(═O)CH₃. In other embodiments, R¹⁴ can be —OC(R¹⁷)₂—O—C(═O)R¹⁸. In an embodiment, each R¹⁷ can be hydrogen. In some embodiments, R¹⁸ can be an unsubstituted or substituted C₁₋₄ alkyl. In an embodiment, R¹⁴ can be —OCH₂—O—C(═O)CH₃, —OCH₂—O—C(═O)(n-butyl) or —OCH₂—O—C(═O)(t-butyl).

Similar to NS¹, in some embodiments, NS² can be selected from an anti-neoplastic agent, an anti-viral agent and an anti-parasitic agent. An exemplary structure of NS² is:

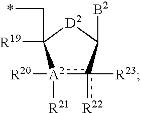

in which A² can be selected from of C (carbon), O (oxygen) and S (sulfur); B² can be an optionally substituted heterocyclic base or a derivative thereof; D² can be C═CH₂ or O (oxygen); R¹⁹ can be selected from hydrogen, azido, —CN, an optionally substituted C₁₋₄ alkyl and an optionally substituted C₁₋₄ alkoxy; R²⁰ can be absent or selected from hydrogen, halogen, hydroxy and an optionally substituted C₁₋₄ alkyl; R²¹ can be absent or selected from hydrogen, halogen, azido, amino and hydroxy; R²² can be selected from hydrogen, halogen, hydroxy, —CN, —NC, an optionally substituted C₁₋₄ alkyl and an optionally substituted C₁₋₄ alkoxy; R²³ can be selected from hydrogen, halogen, hydroxy, —CN, —NC, an optionally substituted C₁₋₄ alkyl, an optionally substituted haloalkyl and an optionally substituted hydroxyalkyl, or when the bond to R²² indicated by [symbol] is a double bond, then R²² and R²³ can be taken together to form a C₁₋₄ alkenyl; and * represents a point of attachment.

In some embodiments, the optionally substituted heterocyclic base or a derivative thereof, B″, can be selected from one of the following:

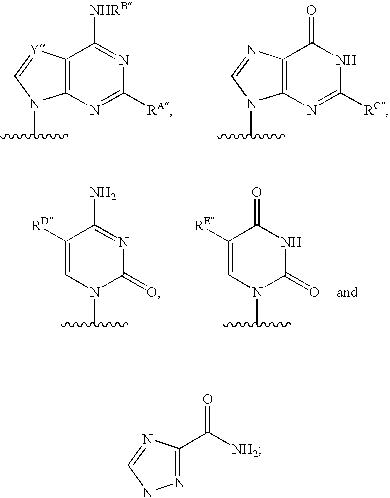

in which R^{A″} can be hydrogen or halogen; R^{B″} can be hydrogen, an optionally substituted C₁₋₄ alkyl, or an optionally substituted C₃₋₈ cycloalkyl; R^{C″} can be hydrogen or amino; R^{D″} can be hydrogen or halogen; R^{E″} can be hydrogen or an optionally substituted C₁₋₄ alkyl; and Y can be N (nitrogen) or CR^{F″}, wherein R^{F″} hydrogen, halogen or an optionally substituted C₁₋₄ alkyl.

Suitable examples of NS² include, but are not limited to, the following:

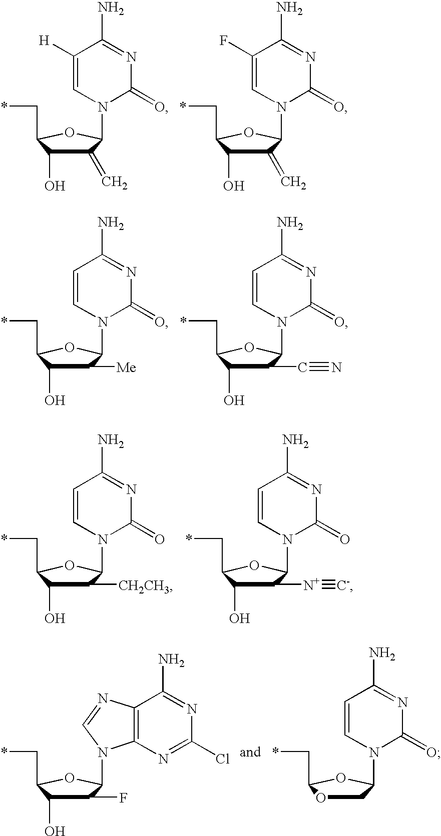

wherein * represents a point of attachment.

Additional examples of NS² include the following:

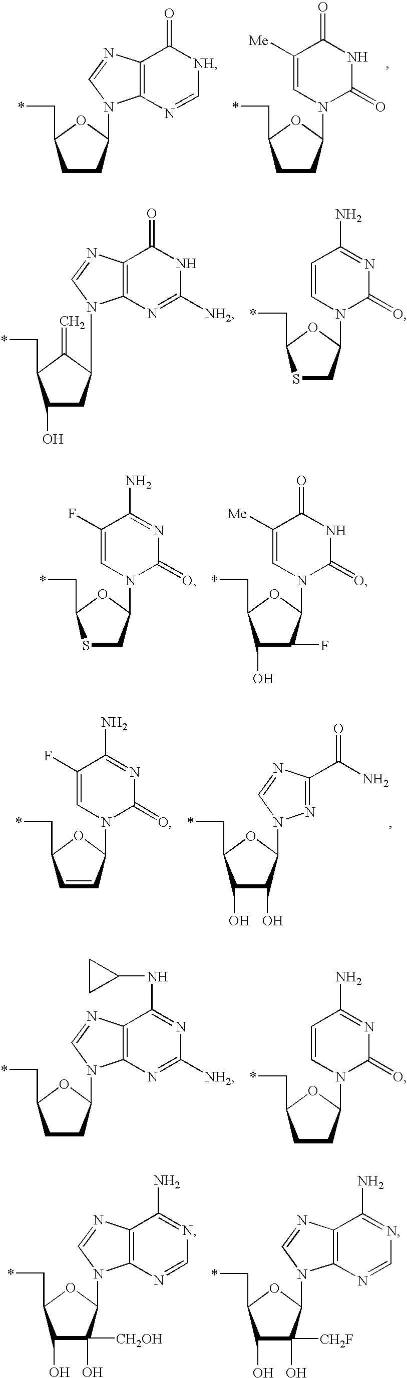

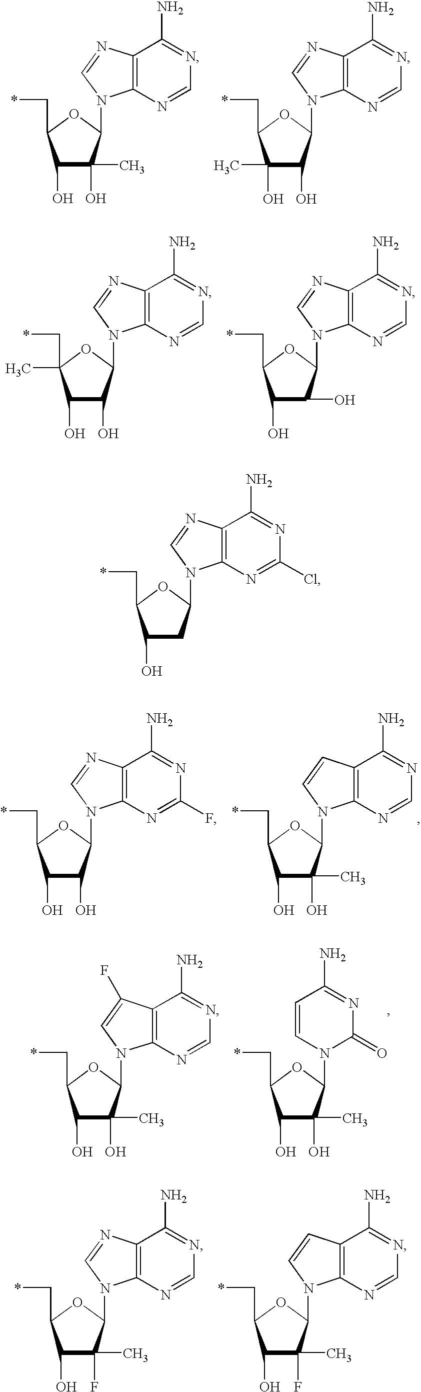

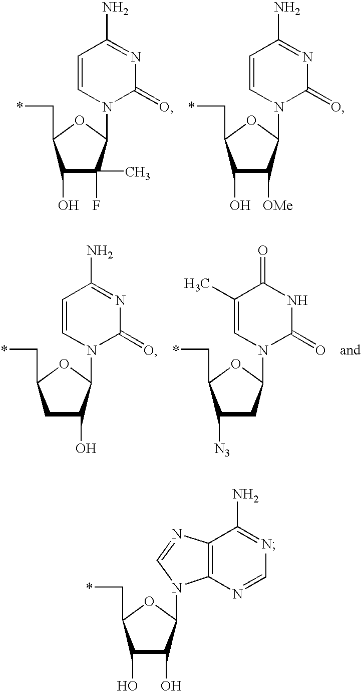

wherein * represents a point of attachment.

As previously stated, NS¹ and/or NS² can be an anti-viral agent, an anti-neoplastic agent and/or an anti-parasitic agent. In an embodiment, the anti-viral agent, anti-neoplastic agent and anti-parasitic agent can be selected to target a particular virus, tumor or parasite, thereby providing a dual mode of action. Upon administration of one or more compounds of Formula (I) to an animal, such as a human, a non-human mammal, a bird, or another animal, the full molecule can activate RNaseL, producing a general anti-viral response, and upon degradation of the compound in vivo, the nucleoside(s) is released, thus generating the particular (generally more specific) therapeutic action (e.g., anti-viral, anti-neoplastic and/or anti-parasitic action) of that moiety. Further, upon release of the nucleoside(s), the intracellular cleavage releases not a nucleoside, but its active, phosphorylated form. This not only makes the nucleoside(s) more immediately available in the intracellular environment, but also bypasses some potential resistance mechanisms such as those described herein. One mechanism that is bypassed is the need for kinase-mediated phosphorylation that both reduces the efficacy of nucleosides in general, but also provides a potential resistance mechanism. This dual-mode of action can provide a powerful benefit in addressing difficult neoplasms, viral infections and/or parasitic infections.

Other embodiments disclosed herein relates to a compound of Formula (Ia) as shown herein, or a pharmaceutically acceptable salt, prodrug or prodrug ester in which R^{1A}, R^{2A}, R^{3A} and R^{4A} can each be

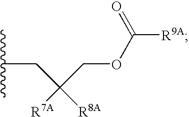

R^{5A} and R^{6A} can be independently selected from hydrogen, —C(═O)R^{10A}, and —C(R^{11A})₂—O—C(═O)R^{12A}; each R^{7A} and each R^{8A} can each be independently selected from—C≡N, an optionally substituted 1-oxoalkyl, an optionally substituted alkoxycarbonyl and an optionally substituted alkylaminocarbonyl; each R^{9A}, each R^{10A}, each R^{11A} and each R^{12A} can each be hydrogen or an optionally substituted C₁₋₄-alkyl; and wherein R^{1A}, R^{2A}, R^{3A} and R^{4A} can be the same or different from each other.

In some embodiments, R^{7A} can be —C≡N. In some embodiments, R^{8A} can be an optionally substituted alkoxycarbonyl, for example, —C(═O)OCH₃. In other embodiments, R^{8A} can be an optionally substituted alkylaminocarbonyl. In an embodiment, R^{8A} can be —C(═O)NHCH₂CH₃. In still other embodiments, R^{8A} can be an optionally substituted 1-oxoalkyl. In an embodiment, the optionally substituted 1-oxoalkyl can be —C(═O)CH₃. In some embodiments, R^{9A} can be an optionally substituted C₁₋₄-alkyl such as methyl, ethyl, n-propyl, isopropyl, n-butyl, iso-butyl and tert-butyl.

In some embodiments, R^{1A}, R^{2A}, R^{3A} and R^{4A} can each be

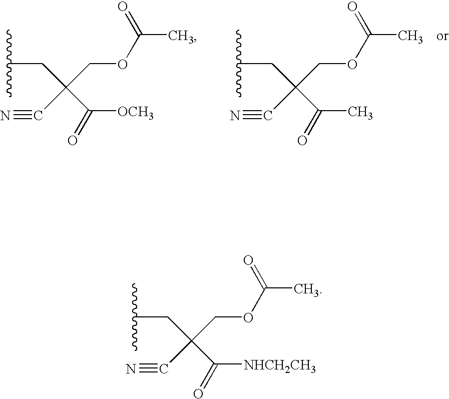

In an embodiment, R^{5A} and R^{6A} can be —C(═O)R^{10A}. In some embodiment, R^{10A} can be unsubstituted or substituted C₁₋₄-alkyl, for example, methyl, ethyl, n-propyl, isopropyl, n-butyl, iso-butyl and tert-butyl. In another embodiment, R^{5A} and R^{6A} can be —C(R^{11A})₂—O—C(═O)R^{12A}. In an embodiment, each R^{11A} can be hydrogen. In some embodiments, R^{12A} can be an unsubstituted or substituted C₁₋₄ alkyl. In an embodiment, R^{12A} can be methyl. In another embodiment, R^{12A} can be n-butyl. In still another embodiment, R^{12A} can be tert-butyl.

In an embodiment, the compound of Formulae (I) and/or (Ia) can be selected from the following:

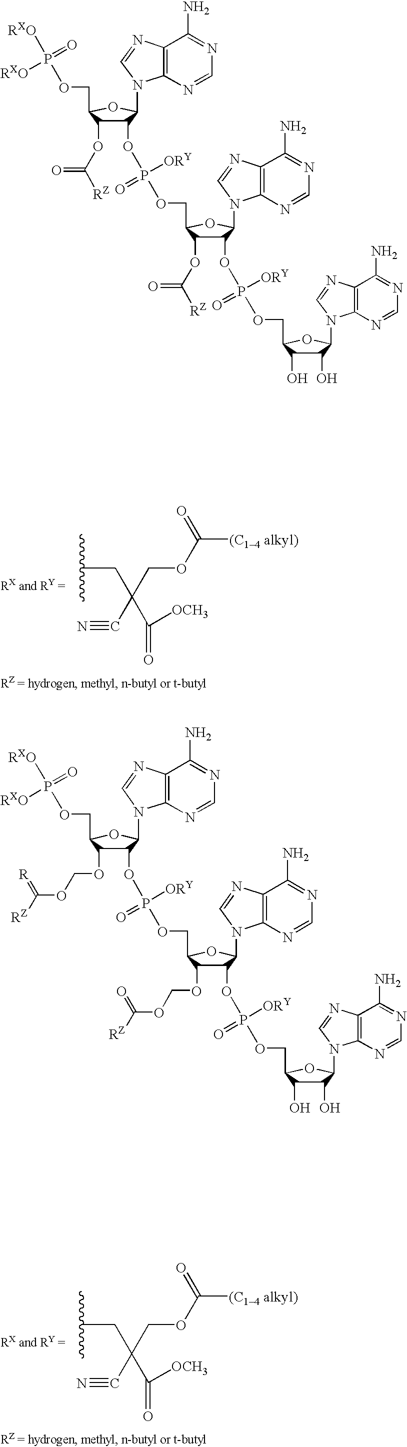

Without asking to be bound by any particular theory, it is believed that neutralizing the charge on the phosphate group facilitates the penetration of the cell membrane by compounds of Formulae (I) and (Ia) by making the compound more lipophilic. Furthermore, it is believed that the 2,2-disubstituted-3-acyloxypropyl groups; for example

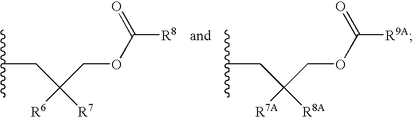

attached to the phosphate impart increased plasma stability to the compounds of Formulae (I) and (Ia) by inhibiting the degradation of the compound. Once inside the cell, the 2,2-disubstituted-3-acyloxypropyl groups attached to the phosphate can be easily removed by esterases via enzymatic hydrolysis of the acyl group. The remaining portions of the group on the phosphate can then be removed by elimination. The general reaction scheme is shown below in Scheme 1. Upon removal of the 2,2-disubstituted-3-acyloxypropyl group, the resulting nucleotide analog possesses a monophosphate. Thus, in contrast to use of trinucleoside compounds, the necessity of an initial intracellular phosphorylation is no longer a prerequisite to obtaining the biologically active phosphorylated form.

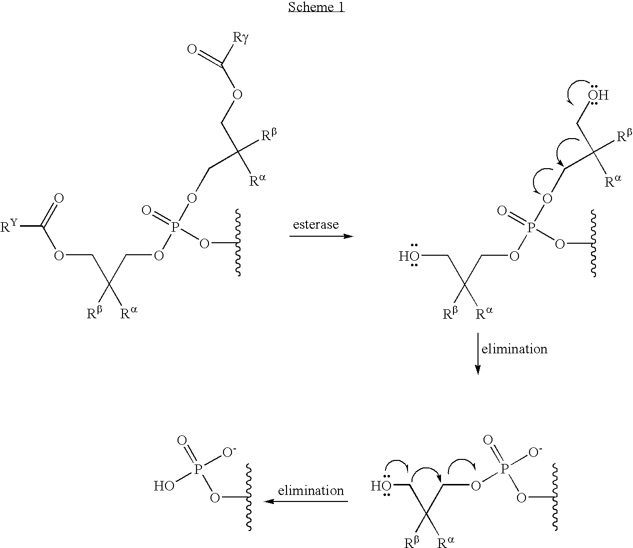

A further advantage of the 2,2-disubstituted-3-acyloxypropyl groups described herein is the rate of elimination of the remaining portion of the 2,2-disubstituted-3-acyloxypropyl group is modifiable. Depending upon the identity of the groups attached to the 2-carbon, shown in Scheme 1 as R^{α} and R^{β}, the rate of elimination may be adjusted from several seconds to several hours. As a result, the removal of the remaining portion of the 2,2-disubstituted-3-acyloxypropyl group can be retarded, if necessary, to enhance cellular uptake but, readily eliminated upon entry into the cell.

When the group on the 3′-position on the middle residue is protected with an acyl or acyloxyalkyl group, the acyl or acyloxyalkyl group can also be removed by esterases via enzymatic hydrolysis of the acyl group followed by elimination of any remaining portion of the group. By varying the group at the 3′-position of the middle residue, the rate of elimination can be modified. It is believed that protecting the 3′-position minimizes and/or inhibits the isomerization of the phosphate on the 2′-position to the 3′-position. Additionally, protection of the 3′-position can reduce the likelihood that the phosphate will be prematurely cleaved off before entry into the cell.

Similarly, when the 3′-position of the 5′-terminal residue is protected, isomerization and premature cleavage of the neighboring 2′-phosphate can be minimized and/or inhibited. Also, when the 3′-position on the 5′-terminal residue is protected, the rate of removal can be modified similarly as discussed above with respect to the 3′-position on the middle residue.

As noted above, the rate of elimination of the groups on the 3′-positions and the phosphates can be adjusted, thus, in some embodiments, the identity of the groups on the phosphates and the 3′-positions can be chosen such that one or more groups on the phosphates are removed before the groups on the 3′-positions. In other embodiments, the identity of the groups on the phosphates and the 3′-positions can be chosen such that at least one group on the phosphates is removed after the groups on the 3′-positions. In an embodiment, the identity of the groups on the phosphates and the 3′-positions can be chosen such that the groups on the internal phosphates attached to the middle and 2′-terminal residues are removed before the groups on the 3′-positions of the middle and 5′-terminal residues. In another embodiment, the identity of the groups on the phosphates and the 3′-positions can be chosen such that the groups on the internal phosphates attached to the middle and 2′-terminal residues are removed before at least one group on the 5′-terminal phosphate and at least one group on the 5′-terminal residue is removed before the groups on the 3′-positions of the middle and 5′-terminal residues. In still another embodiment, the identity of the groups on the phosphates and the 3′-positions can be chosen such that the groups on the internal phosphates attached to the middle and 2′-terminal residues are removed before the groups on the 5′-terminal phosphate which in turn are removed before the groups on the 3′-positions of the middle and 5′-terminal residues.

While not wanting to be bound by any particular theory, it is believed that by protecting the phosphate groups and the 3′-positions of the middle and 5′-terminal residues, the breakdown of the trimer can be adjusted. This in turn can enhance cellular uptake and assist in maintaining the balance between unwanted viral RNA and native cellular RNA.

### Synthesis

Compounds of Formulae (I) and (Ia) and those described herein may be prepared in various ways. General synthetic routes to the compounds of Formulae (I) and (Ia), and the starting materials used to synthesize the compounds of Formulae (I) and (Ia) are shown in Schemes 2a-2f. The routes shown are illustrative only and are not intended, nor are they to be construed, to limit the scope of this invention in any manner whatsoever. Those skilled in the art will be able to recognize modifications of the disclosed synthesis and to devise alternate routes based on the disclosures herein; all such modifications and alternate routes are within the scope of this invention.

The hydroxy precursors,

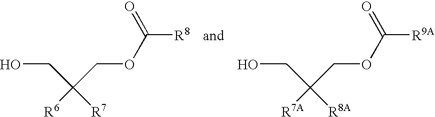

in which R⁶, R⁷, R⁸, R^{7A}, R^{8A} and R^{9A} are the same as described herein, of the 2,2-disubstituted-3-acyloxypropyl groups can be synthesized according in a manner similar to those described in the following articles. Ora, et al., J. Chem. Soc. Perkin Trans. 2, 2001, 6, 881-5; Poijärvi, P. et al., Helv. Chim. Acta. 2002, 85, 1859-76; Poijärvi, P. et al., Lett. Org. Chem., 2004, 1, 183-88; and Poijärvi, P, et al., Bioconjugate Chem., 2005 16(6), 1564-71, all of which are hereby incorporated by reference in their entireties.

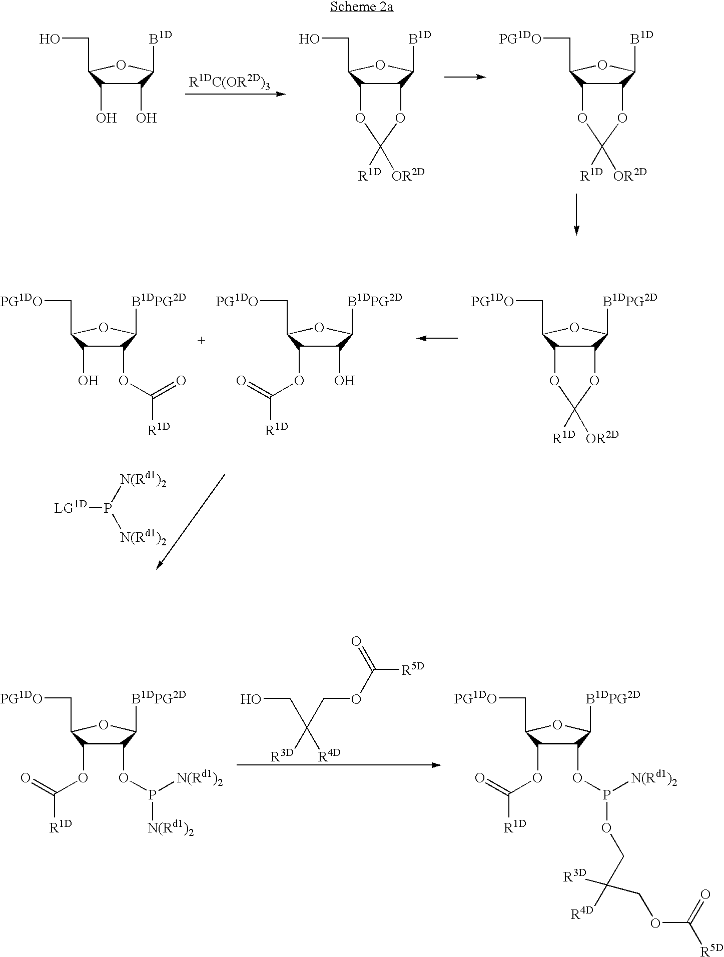

One example for synthesizing a nucleoside compound in which the 3′-position has an oxyacyl group, for example, —OC(═O)R⁹ and —OC(═O)R¹⁶, is shown in Scheme 2a. A R^{1D}C(OR^{2D})₃ moiety, in which R^{1D} can be hydrogen or an optionally substituted C₁₋₄ alkyl and R^{2D} can be an optionally substituted C₁₋₄ alkyl, can be added to a nucleoside using the methods described in Griffin et al., Tetrahedron (1967), 23 2301-13, which is hereby incorporated by reference in its entirety. The 5′-OH of the nucleoside can be protected with an appropriate protecting group. One suitable group is a silyl ether protecting group. Exemplary silyl ether protecting groups are described herein. The heterocyclic base or heterocyclic base derivative, represented by B^{1D}, on the nucleoside can also be protected using an appropriate protecting group. An exemplary protecting group for the heterocyclic base or heterocyclic base derivative is a triarylmethyl protecting group such as those described herein. The di-ether ring can be opened using methods known to those skilled in the art, for example, using an acid. The ring opening can lead to two isomers shown above in which the oxycarbonylalkyl group is on either the 2′- or 3′-position. If desired, these isomers can be separated using methods known to those skilled in the art. Alternatively, a compound having the structure:

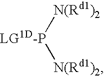

can be added to the free 3′-OH or 2′-OH positions. In the compound having the structure:

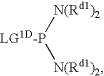

R^{d1} can be an optionally substituted C₁₋₄ alkyl; and LG^{1D} can be an appropriate leaving group such as a halogen. After addition of the compound having the structure:

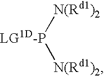

the resulting two isomers having a phosphoamidite at either the 2′- or 3′-position can be separated using methods known to those skilled in the art. A hydroxy precursor having the structure:

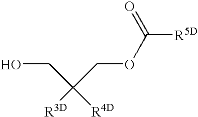

can be added to the phosphoamidite to form the desired nucleoside compound with a 3′-position having an oxycarbonylalkyl group. R^{3D} and R^{4D} of the hydroxy precursor can be each independently selected from-C≡N, an optionally substituted 1-oxoalkyl, an optionally substituted alkoxycarbonyl and an optionally substituted alkylaminocarbonyl; and R^{5D} can be hydrogen or an optionally substituted C₁₋₄-alkyl. If desired, an activator such as those described herein can be used to facilitate the reaction.

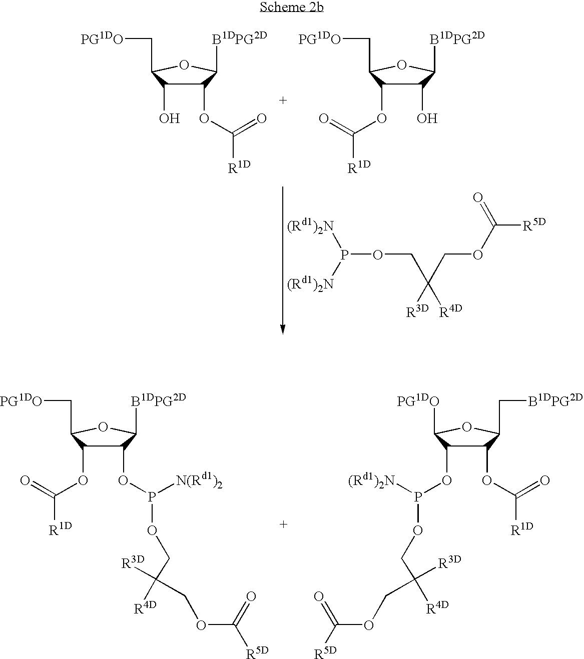

Another example for synthesizing a nucleoside compound in which the 3′-position has an oxyacyl group, for example, —OC(═O)R⁹ and —OC(═O)R¹⁶, is shown in Scheme 2b. The two isomers formed after the di-ether ring opening step in Scheme 2a can be reacted with a compound having the structure of

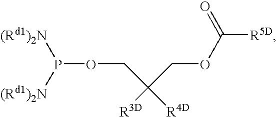

wherein R^{3D}, R^{4D}, R^{5D} and R^{d1} can be the same as described in Scheme 2a. The two resulting isomers can be separated and the desired nucleoside compound with the 3′-position having an oxycarbonylalkyl group can be isolated using methods known to those skilled in the art.

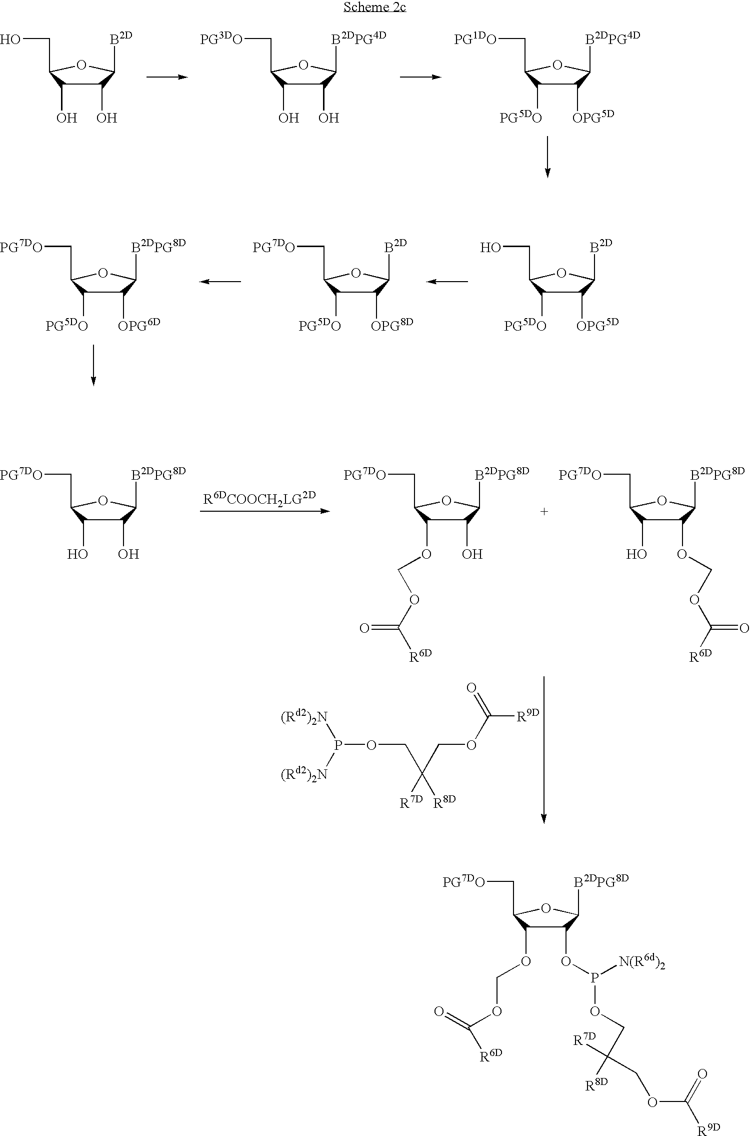

In Scheme 2c, an example for synthesizing a nucleoside compound in which the 3′-position has an oxyalkyloxyacyl group, for example, —OC(R¹⁰)₂—O—C(═O)R¹¹ and —OC(R^{11A})₂—O—C(═O)R^{12A}, is shown. The 5′-OH and the heterocyclic base or heterocyclic base derivative, represented by B^{2D}, on the nucleoside can be protected using appropriate protecting groups, for example, triarylmethyl protecting groups. Exemplary triarylmethyl protecting groups are described herein. The protecting groups on the 5′-OH and the heterocyclic base or heterocyclic base derivative can be the same or different. The 2′-OH and 3′-OH can also be protected with protecting groups. In some embodiments, the protecting groups used on the 2′-OH and 3′-OH can be different from those on the 5′-OH and the heterocyclic base or heterocyclic base derivative. In an embodiment, the 2′-OH and 3′-OH can be protected with levulinoyl groups. The protecting groups on the 5′-OH and the heterocyclic base or heterocyclic base derivative can then be removed using methods known to those skilled in the art. For example, if the protecting groups on the 5′-OH and the heterocyclic base or heterocyclic base derivative are both triarylmethyl protecting groups, both can be removed using an appropriate acid (e.g., acetic acid) or a zinc dihalide. The 5′-OH can be then reprotected with another protecting group. The protecting group can be the same or different from the first protecting group on the 5′-OH. In an embodiment, PG^{7D} can be a silyl ether protecting group, such as those described herein. In some embodiment, PG^{1D} can be a triarylmethyl protecting group and PG^{7D} can be a silyl ether protecting group. The heterocyclic base or heterocyclic base derivative, represented by B^{2D} can also be reprotected with an appropriate protecting group. The protecting group can be the same or different from the first protecting group on the heterocyclic base or heterocyclic base derivative. In an embodiment, PG^{8D} can be triarylmethyl protecting group such as those described herein. In some embodiments, PG^{2D} and PG^{8D} can both be a triarylmethyl protecting group. The protecting groups on the 2′- and 3′-positions can then be removed using methods known to those skilled in the art. In an embodiment, PG^{5D} and PG^{6D} can be levulinoyl groups that can be removed with an appropriate reagent. One exemplary reagent is using hydrazinium acetate. After removal of the levulinoyl groups, a compound of formula R^{6D}COOCH₂LG^{2D}, wherein R^{6D} can be hydrogen or an optionally substituted C₁₋₄ alkyl and LG^{2D} can be an appropriate leaving group, can be added non-selectively as shown above in Scheme 2c. If desired, the two resulting isomers can be separated using methods known to those skilled in the art. Alternatively, a compound of Formula

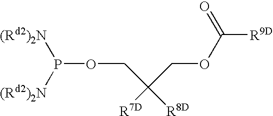

can be added to the free 2′-OH and 3′-OH groups. In the compound of Formula

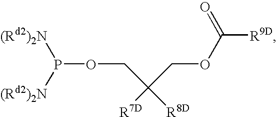

R^{7D} and R^{8D} can be each independently selected from-C“N, an optionally substituted 1-oxoalkyl, an optionally substituted alkoxycarbonyl and an optionally substituted alkylaminocarbonyl; R^{9D} can be hydrogen or an optionally substituted C₁₋₄-alkyl; and each R^{d2} can be an optionally substituted C₁₋₄-alkyl. To facilitate the reaction, an activator can be used. Suitable activators are described herein. The resulting two isomers can be separated and the desired nucleoside compound with the 3′-position having an oxyalkyloxyacyl group can be isolated using methods known to those skilled in the art.

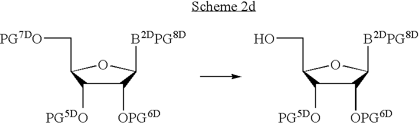

One method for synthesizing a nucleoside compound with a free 5′-OH is shown in Scheme 2d. A nucleoside with a protected heterocyclic base or protected heterocyclic base derivative, and with the 2′-, 3′- and 5′-positions protected can be formed as described above in Scheme 2c. The protecting group on the 5′-position can be removed using one or methods known to those skilled in the art. For example, if the protecting group represented by PG^{7D} is a silyl ether protecting group, the silyl ether protecting group can be removed using a tetra(alkyl)ammonium halide (e.g., tetra(t-butyl)ammonium fluoride). The protecting groups on the nucleoside compound can be chosen such that PG^{7D} can be removed without removing one or more protecting group selected from PG^{5D}, PG^{6D} and PG^{8D}.

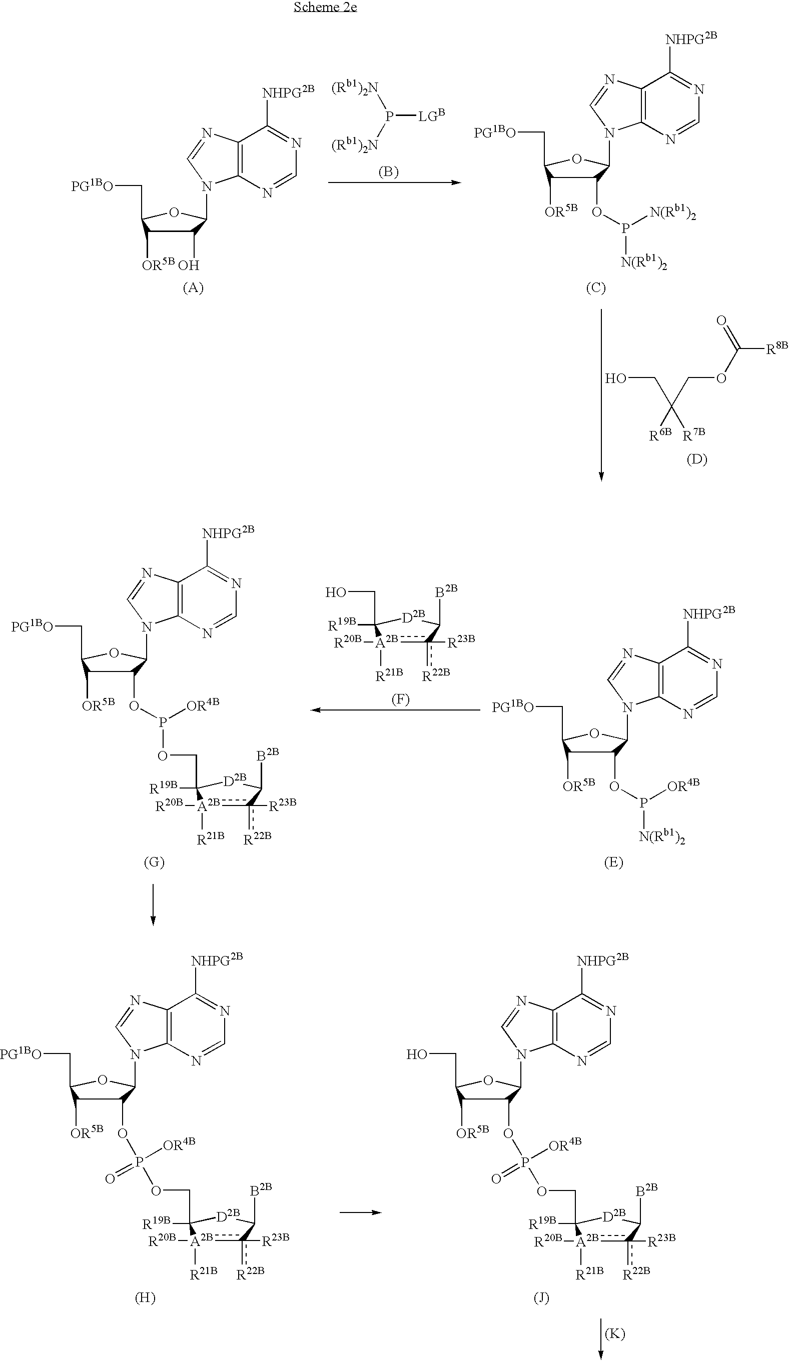

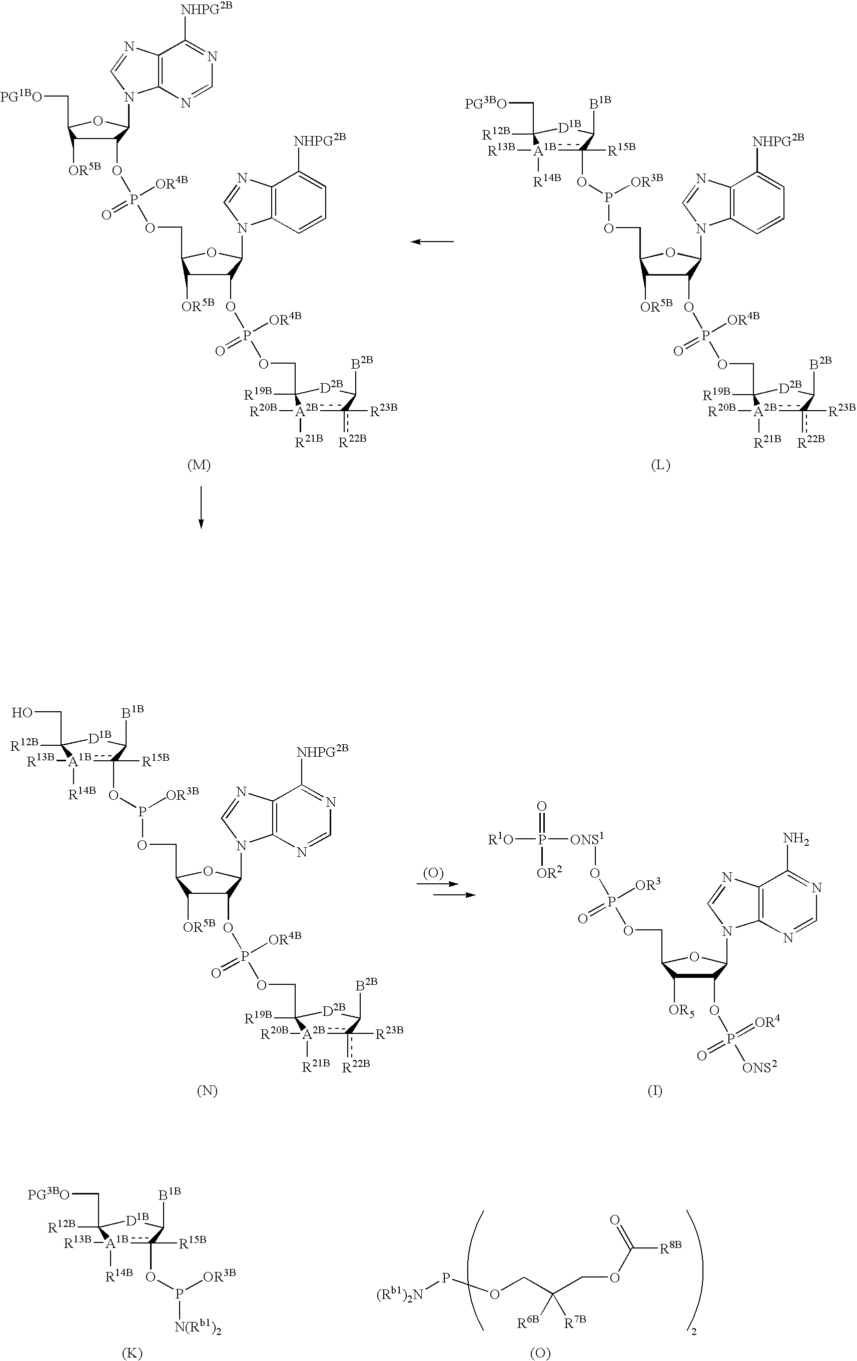

One embodiment disclosed herein relates to a method of synthesizing a compound of Formula (I) that includes the transformations shown in Scheme 2e. In Scheme 2e, R^{1B}, R^{2B}, R^{3B}, R^{4B}, R^{5B}, R^{6B}, R^{7B}, R^{8B}, R^{9B}, R^{10B} and R^{11B} can be the same as R¹, R², R³, R⁴, R⁵, R⁶, R⁷, R⁸, R⁹, R¹⁰ and R¹¹, respectively, as described above with respect to a compound of Formula (I). PG^{1B}, PG^{2B} and PG^{3B} represent appropriate protecting groups. In some embodiments, PG^{1B} can be a silyl ether. Exemplary silyl ethers include, but are not limited to, trimethylsilyl (TMS), tert-butyldimethylsilyl (TBDMS), triisopropylsilyl (TIPS) and tert-butyldiphenylsilyl (TBDPS). In an embodiment, PG^{2B} can be a triarylmethyl protecting group. Examples of suitable triarylmethyl protecting groups, include but are not limited to, trityl, monomethoxytrityl (MMTr), 4,4′-dimethoxytrityl (DMTr), 4,4′,4″-trimethoxytrityl (TMTr), 4,4′,4″-tris-(benzoyloxy)trityl (TBTr), 4,4′,4″-tris (4,5-dichlorophthalimido) trityl (CPTr), 4,4′,4″-tris (levulinyloxy) trityl (TLTr), p-anisyl-1-naphthylphenylmethyl, di-o-anisyl-1-naphthylmethyl, p-tolyldipheylmethyl, 3-(imidazolylmethyl)-4,4′-dimethoxytrityl, 9-phenylxanthen-9-yl (Pixyl), 9-(p-methoxyphenyl) xanthen-9-yl (Mox), 4-decyloxytrityl, 4-hexadecyloxytrityl, 4,4′-dioctadecyltrityl, 9-(4-octadecyloxyphenyl)xanthen-9-yl, 1,1′-bis-(4-methoxyphenyl)-1′-pyrenylmethyl, 4,4′,4″-tris-(tert-butylphenyl)methyl (TTTr) and 4,4′-di-3,5-hexadienoxytrityl.

A compound of Formula C can be produced by forming a phosphoamidite at the 2′-position of a compound of Formula A by reacting a compound of Formula B with the 2′-OH of a compound of Formula A to form a compound of Formula C. In an embodiment, each R^{b1} can be independently an optionally substituted C₁₋₄ alkyl, and LG^{B} can be a suitable leaving group. In an embodiment, the leaving group on a compound of Formula B can be a halogen. One benefit of having the other hydroxy groups on a compound of Formula A and any amino groups attached to the heterocyclic base or derivative thereof and/or a NH group(s) present in a ring of the heterocyclic base or derivative thereof protected is that the addition of a compound of Formula B can be directed to the 2′-position of a compound of Formula A. Furthermore, the protecting groups on the hydroxy groups and any amino groups attached to the heterocyclic base or derivative thereof and/or a NH group(s) present in a ring of the heterocyclic base or derivative thereof can block undesirable side reactions that may occur during later synthetic transformations. Minimization of unwanted side compound can assist in the separation and isolation of the desired compound(s).

A R^{4B} moiety can be added to a compound of Formula C by reacting a compound of Formula C with a compound of Formula D to form a compound of Formula E. As shown in Scheme 2e, the R^{4B} moiety can add to the phosphoamidite of a compound of Formula C. In some embodiments, an activator can be used to facilitate the addition of the R^{4B} moiety. An exemplary activator is a tetrazole such as benzylthiotetrazole. The tetrazole can protonate the nitrogen of the phosphoamidite making it susceptible to nucleophilic attack by the R^{4B} moiety. Additional activators that can be used are disclosed in Nurminen, et al., J. Phys. Org. Chem., 2004, 17, 1-17 and Michalski, J. et al., Stated of the Art. Chemical Synthesis of Biophosphates and their Analogues via P^{III} Derivatives, Springer Berlin (2004) vol. 232, pages 43-47; which is hereby incorporated by reference for their disclosure of additional activators.

A nucleoside, a nucleoside analog a protected nucleoside or a protected nucleoside analog can be added to a compound of Formula E by reacting a compound of Formula E with a nucleoside, a nucleoside analog a protected nucleoside or a protected nucleoside analog to form a compound of Formula G. The nucleoside, the nucleoside analog the protected nucleoside or the protected nucleoside analog can add to the phosphorous on a compound of Formula E through its free 5′-OH or equivalent free hydroxy group. In some embodiments, the nucleoside, the nucleoside analog, the protected nucleoside or the protected nucleoside analog can have the structure of a compound of Formula F in which R^{19B} can be selected from hydrogen, azido, —CN, an optionally substituted C₁₋₄ alkyl and an optionally substituted C₁₋₄ alkoxy; R^{20B} can be absent or selected from hydrogen, halogen, hydroxy and an optionally substituted C₁₋₄ alkyl; R^{21B} can be absent or selected from hydrogen, halogen, azido, amino, hydroxy and —OPG^{4B}; R^{22B} can be selected from hydrogen, halogen, hydroxy, —CN, —NC, an optionally substituted C₁₋₄ alkyl, an optionally substituted C₁₋₄ alkoxy and —OPG^{5B}; R^{23B} can be selected from hydrogen, halogen, hydroxy, —CN, —NC, an optionally substituted C₁₋₄ alkyl, an optionally substituted haloalkyl and an optionally substituted hydroxyalkyl, or when the bond to R^{22B} indicated by [symbol] is a double bond, then R^{22B} and R^{23B} can be taken together to form a C₁₋₄ alkenyl; A^{2B} can be selected from C (carbon), O (oxygen) and S (sulfur); D^{2B} can be C═CH₂ or O (oxygen); B^{2B} can be selected from an optionally substituted heterocyclic base, an optionally substituted heterocyclic base derivative, an optionally substituted protected heterocyclic base, and an optionally substituted protected heterocyclic base derivative; and PG^{4B} and PG^{5B} can each be a protecting group. To facilitate the reaction, an activator, such as those previously described, can be used. In some embodiments, PG^{4B} can be a levulinoyl group. In some embodiments, PG^{5B} can be a levulinoyl group.

The phosphite of a compound of Formula G can be oxidized to a phosphate moiety to form a compound of Formula H. In an embodiment, the oxidation can be carried out using iodine as the oxidizing agent and water as the oxygen donor.

The protecting group moiety, PG^{1B}, can be removed to form a compound of Formula J. In an embodiment, PG^{1B} can be a silyl ether which can be removed with a tetra(alkyl)ammonium halide such as tetra(t-butyl)ammonium fluoride. In some embodiments, PG^{1B} can be selectively removed such that PG^{1B} is removed without removing PG^{2B} and/or any protecting groups on the amino groups attached to the heterocyclic base or derivative thereof and/or on the NH group(s) present in a ring of the heterocyclic base or derivative thereof. For example, PG^{1B} can be removed using a reagent such as a tetra(alkyl)ammonium halide that does not remove PG^{2B} and/or any protecting groups on the amino groups attached to the heterocyclic base or derivative thereof and/or on the NH group(s) present in a ring of the heterocyclic base or derivative thereof.

A nucleoside, a nucleoside analog, a protected nucleoside or a protected nucleoside analog can be added to a compound of Formula J by reacting a compound of Formula J with a nucleoside, a nucleoside analog, a protected nucleoside or a protected nucleoside analog to form a compound of Formula L. In some embodiments, the nucleoside, the nucleoside analog, the protected nucleoside or the protected nucleoside analog can have the structure of a compound of Formula K in which R^{12B} can be selected from hydrogen, azido, —CN, an optionally substituted C₁₋₄ alkyl and an optionally substituted C₁₋₄ alkoxy; R^{13B} can be absent or selected from hydrogen, halogen, hydroxy and an optionally substituted C₁₋₄ alkyl; R^{14B} can be absent or selected from hydrogen, halogen, azido, amino, hydroxy, —OC(═O)R^{16B}, and —OC(R^{17B})₂—O—C(═O)R^{18B}; R^{15B} can be selected from hydrogen, halogen, hydroxy, —CN, —NC, an optionally substituted C₁₋₄ alkyl, an optionally substituted haloalkyl and an optionally substituted hydroxyalkyl; each R^{16B}, each R^{17B} and each R^{18B} can be independently hydrogen or an optionally substituted C₁₋₄-alkyl; A^{1B} can be selected from C (carbon), O (oxygen) and S (sulfur); D^{1B} can be C═CH₂ or O (oxygen); B^{1B} can be selected from an optionally substituted heterocyclic base, an optionally substituted heterocyclic base derivative, an optionally substituted protected heterocyclic base, and an optionally substituted protected heterocyclic base derivative; R^{3B} can be the same as R³ as described with respect to a compound of Formula (I), each R^{b1} can be an optionally substituted C₁₋₄ alkyl and PG^{3B} can be a protecting group. The addition of the nucleoside, the nucleoside analog, the protected nucleoside and the protected nucleoside analog can be facilitated by using activator such as those described above. In some embodiments, PG^{3B} can be a silyl ether group.

In an embodiment, B^{1B} and B^{2B} can be each independently selected from

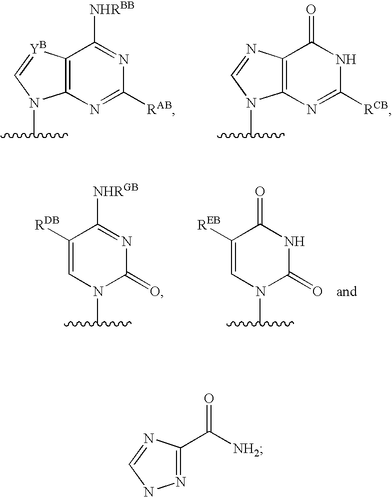

in which R^{AB} can be hydrogen or halogen; R^{BB} can be hydrogen, an optionally substituted C₁₋₄ alkyl, an optionally substituted C₃₋₈ cycloalkyl or a protecting group; R^{CB} can be hydrogen or amino; R^{DB} can be hydrogen or halogen; R^{EB} can be hydrogen or an optionally substituted C₁₋₄ alkyl; Y^{B} can be N (nitrogen) or CR^{FB}, wherein R^{FB} hydrogen, halogen or an optionally substituted C₁₋₄ alkyl; and R^{GB} can be a protecting group. In an embodiment, one or both of R^{BB} and R^{GB} can be a triarylmethyl protecting group such as those described previously. In an embodiment, B^{1B} and B^{2B} can be the same. In another embodiment, B^{1B} and B^{2B} can be different.

The phosphite of a compound of Formula L can be oxidized to a phosphate to form a compound of Formula M. In some embodiments, the oxidation can be carried out using iodine as the oxidizing agent and water as the oxygen donor.

The protecting group represented by PG^{3B} can be removed using methods known to those skilled in the art to form a compound of Formula N: For example, in some embodiments, when PG^{3B} is a silyl ether group, PG^{3B} can be removed using a tetra(alkyl)ammonium halide. One exemplary tetra(alkyl)ammonium halide is tetra(t-butyl)ammonium fluoride. In some embodiments, PG^{3B} can be selectively removed such that PG^{3B} is removed without removing PG^{2B} and/or any protecting groups on the amino groups attached to the heterocyclic base or derivative thereof and/or on the NH group(s) present in a ring of the heterocyclic base or derivative thereof. For example, PG^{3B} can be removed using a reagent such as a tetra(alkyl)ammonium halide that does not remove PG^{2B} and/or any protecting groups on the amino groups attached to the heterocyclic base or derivative thereof and/or on the NH group(s) present in a ring of the heterocyclic base or derivative thereof.

A compound of Formula O can be added to the 5′-OH on a compound of Formula N. In some embodiments, each R^{b1} can be independently an optionally substituted C₁₋₄ alkyl; and each R^{6B}, each R^{7B} and each R^{8B} can be the same as R⁶, R⁷ and R⁸ as described herein with respect to a compound of Formula (I).

The protecting group represented by PG^{2B}, any additional protecting groups present attached to the heterocyclic bases of NS^{1B} and NS^{2B}, and any protecting group on the oxygens attached as hydroxy groups to the 2′ and 3′-positions of NS^{1B} and NS^{2B} can be removed using methods known to those skilled in the art to form a compound of Formula (I). In an embodiment, PG^{2B} can be removed with an acid such as acetic acid or a zinc dihalide, such as ZnBr₂. In some embodiments, the heterocyclic bases or heterocyclic base derivaties such as B^{1B} and B^{2B} can be protected with triarylmethyl protecting groups which can removed with an acid (e.g., acetic acid). For example, any amino groups attached to one of the rings of the heterocyclic base or heterocyclic base derivative can be protected with one or more protecting groups such as triarylmethyl protecting groups. In some embodiment, levulinoyl protecting groups can be attached to one or more oxygens of NS^{2B}. In an embodiment, the levulinoyl protecting groups can be removed with hydrazinium acetate. In other embodiment, silyl ether protecting groups can be attached to one or more oxygens of NS^{2B}. In an embodiment, the silyl ether groups can be removed using a tetraalkylammonium halide (e.g., tetrabutylammonium fluoride). In some embodiments, the protecting groups on the oxygens attached to the 2′ and 3′-positions of NS^{2B}, if present, can be removed selectively. For example, protecting groups on the oxygens attached to the 2′ and 3′-positions can be removed without removing any protecting groups attached to the heterocyclic bases or the heterocyclic base derivatives of NS^{1B} and NS^{2B}. Alternatively, any protecting groups on the heterocyclic bases or heterocyclic base derivatives of NS^{1B} and NS^{2B} can be selectively removed such that the protecting groups on the heterocyclic bases or heterocyclic base derivatives of NS^{1B} and NS^{2B} can be removed without removing any protecting groups on the oxygens attached to the 2′ and 3′-positions of NS^{2B}. In an embodiment, protecting groups on the oxygens attached to the 2′ and 3′-positions of NS^{2B}, if present, can be removed before removing any protecting groups on the heterocyclic bases or heterocyclic base derivatives of NS^{1B} and NS^{2B}. In another embodiment, protecting groups on the oxygens attached to the 2′ and 3′-positions of NS^{2B}, if present, can be removed after removing any protecting groups on the heterocyclic bases or heterocyclic base derivatives of NS^{1B} and NS^{2B}. In some embodiments, protecting groups on the oxygens attached to the 2′ and 3′-positions of NS^{2B}, if present, can be removed almost simultaneously. In other embodiments, protecting groups on the oxygens attached to the 2′ and 3′-positions of NS^{2B}, if present, can be removed sequentially. In some embodiments, protecting groups on the heterocyclic bases or heterocyclic base derivatives of NS^{1B} and NS^{2B} can be removed almost simultaneously. In other embodiments, protecting groups on the heterocyclic bases of NS^{1B} and NS^{2B} can be removed sequentially.

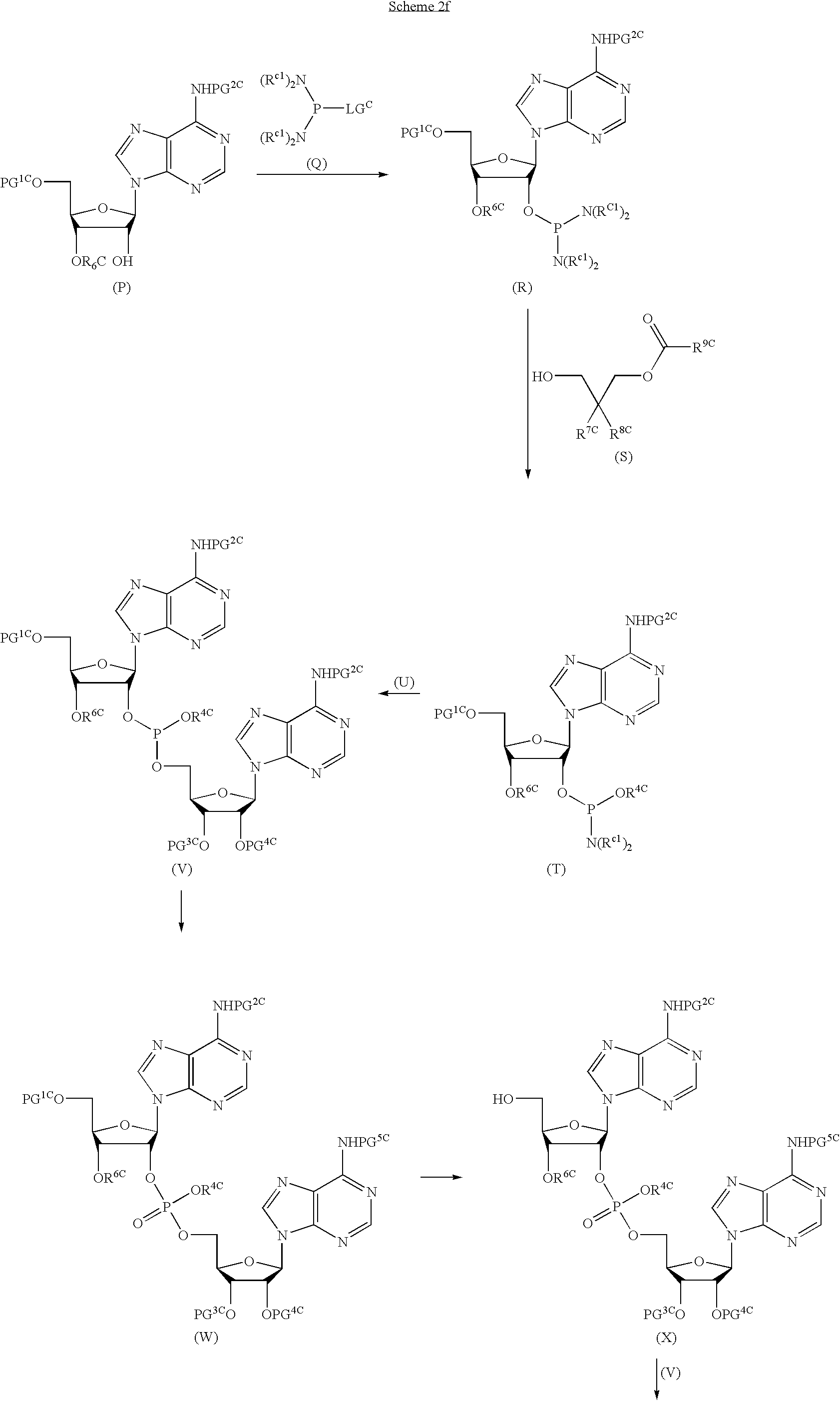

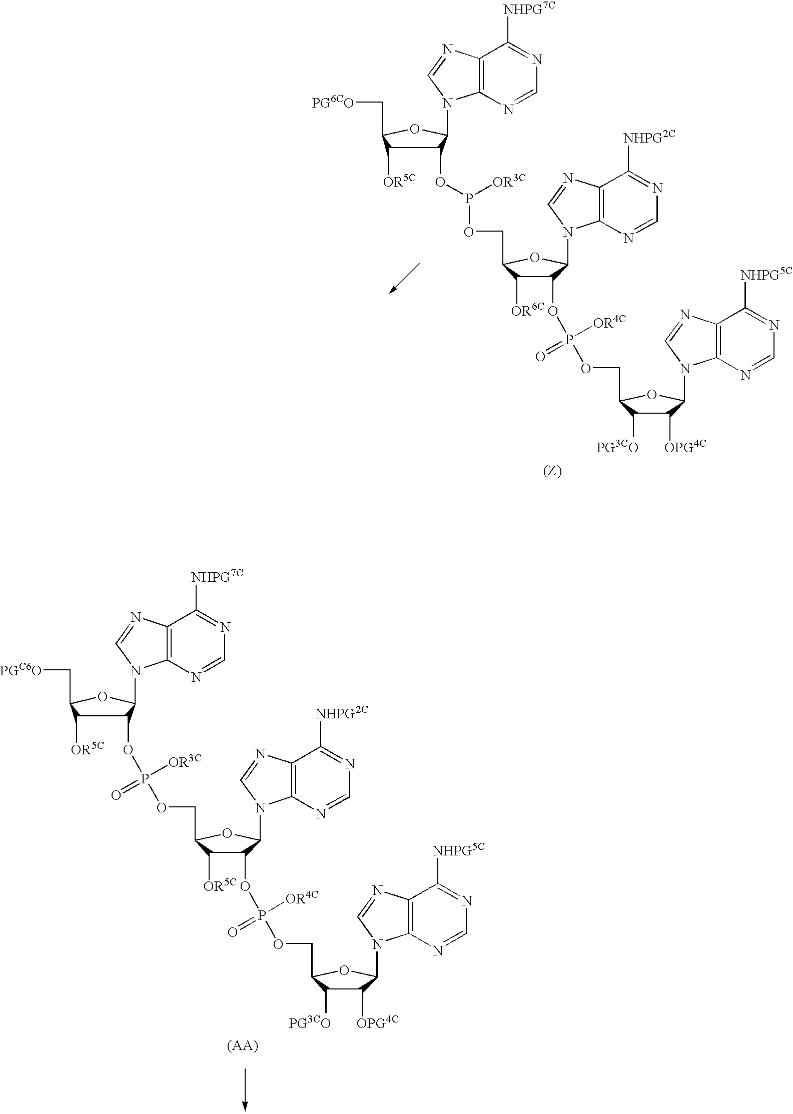

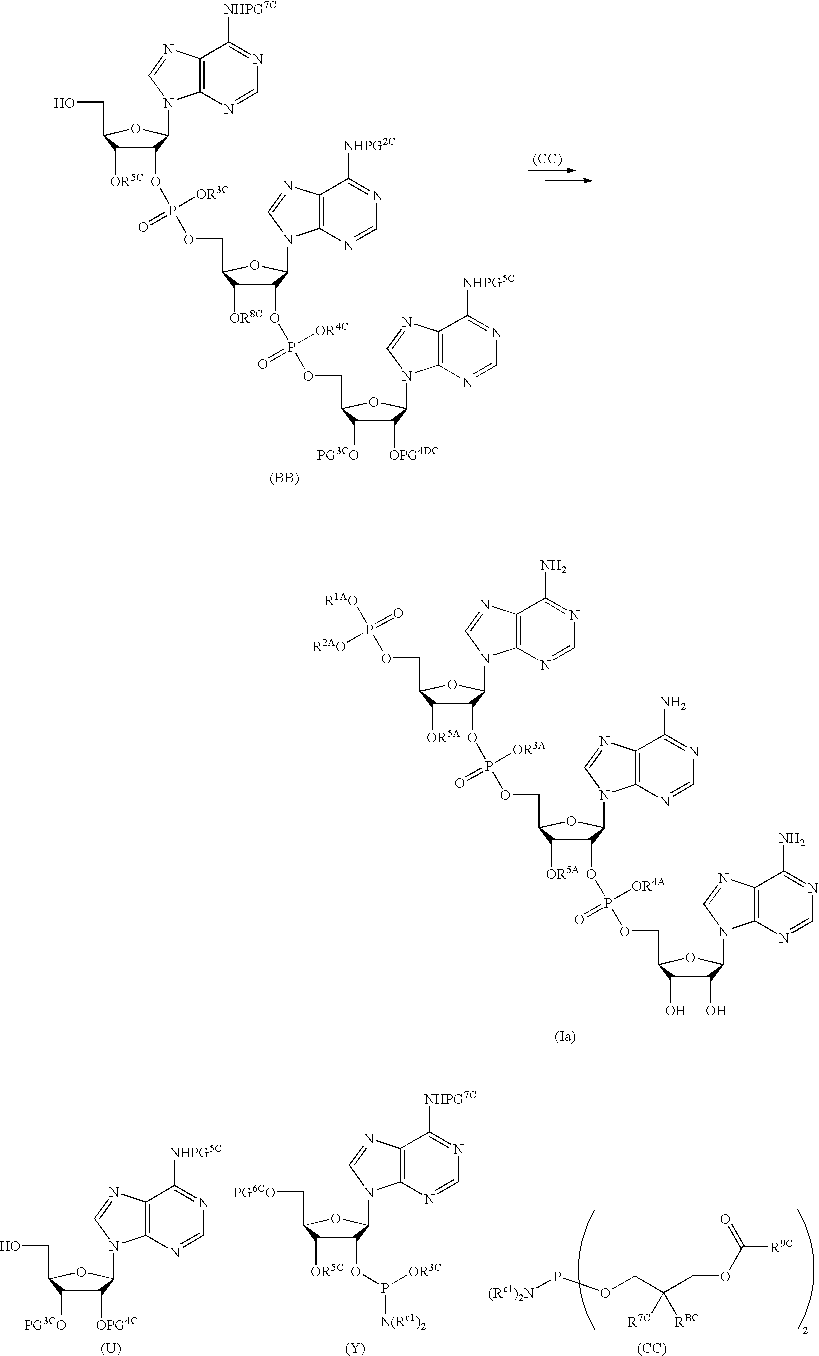

An embodiment described herein relates to a method of synthesizing a compound of Formula (Ia) as shown in Scheme 2f. In Scheme 2f, R^{1C}, R^{2C}, R^{3C}, R^{4C}, R^{5C}, R^{6C}, R^{7C}, R^{8C}, R^{9C}, R^{10C}, R^{11C} and R^{12C} can be the same as R^{1A}, R^{2A}, R^{3A}, R^{4A}, R^{5A}, R^{6A}, R^{7A}, R^{8A}, R^{9A}, R^{10A}, R^{11A} and R^{12A} respectively, as described above with respect a compound of Formula (Ia). PG^{1C}, PG^{2C}, PG^{3C}, PG^{4C}, PG^{5C}, PG^{6C} and PG^{7C} represent appropriate protecting groups. In some embodiments, PG^{1C} can be a silyl ether. Examples of suitable silyl ethers are described herein. In an embodiment, PG^{2C} can be a triarylmethyl protecting groups. Exemplary triarylmethyl protecting groups are disclosed herein.

As shown in Scheme 2f, a phosphoamidite can be formed at the 2′-position of a compound of Formula P by reacting a compound of Formula Q with the 2′-OH of a compound of Formula P to form a compound of Formula R. In an embodiment, each R^{c1} can be independently an optionally substituted C₁₋₄ alkyl, and LG^{C} can be a suitable leaving group. In some embodiments, LG^{C} can be a halogen. Benefits of having PG^{1C} and PG^{2C} present include, but are not limited, the addition of a compound of Formula Q can be directed to the 2′-position of a compound of Formula P and the number of undesirable side reactions that may occur during later synthetic transformations can be minimized. As a result, the separation and isolation of the desired compound(s) can be made easier.

A R^{4C} moiety can be added to the phosphoamidite on a compound of Formula R by reacting a compound of Formula R with a compound of Formula S to form a compound of Formula T. In some embodiments, an activator such as those described can be used to facilitate the addition of a compound of Formula S to a compound of Formula R.

A compound of Formula U can be added to a compound of Formula T to form a compound of Formula V. As shown in Scheme 2f, a compound of Formula U can be added to a compound of Formula T through its free 5′-OH group. If desired, an activator can be used to facilitate this reaction. In some embodiments, PG^{3C} on a compound of Formula U can be a levulinoyl group. In some embodiments, PG^{4C} on a compound of Formula U can be a levulinoyl group. In an embodiment, PG^{5C} can be a triarylmethyl protecting group. A non-limiting list of triarylmethyl protecting groups is provided herein.

The phosphite of a compound of Formula V can be oxidized to a phosphate. The phosphite can be oxidized using methods known to those skilled in the art. One exemplary method is using iodine as an oxidizing agent and water as the oxygen source.

The protecting group, PG^{1C}, can be removed using methods known to those skilled in the art to form a compound of Formula X. For example, when PG^{1C} is a silyl ether group, PG^{1C} can be removed using a tetra(alkyl)ammonium halide such as tetra(t-butyl)ammonium fluoride. In some embodiments, PG^{1C} can be selectively removed such that PG^{1C} is removed without removing one or more selected from PG^{2C}, PG^{3C}, PG^{4C} and PG^{5C}. For example, PG^{1C} can be removed using a reagent such as a tetra(alkyl)ammonium halide that does not remove PG^{2C}, PG^{3C}, PG^{4C} and/or PG^{5C}.

A compound of Formula Y can be added to a compound of Formula X to form a compound of Formula Z. As shown in Scheme 2f, a compound of Formula Y can be added to a compound of Formula X through the phosphorous on the compound of Formula Y. As in previous steps, in some embodiments, an activator can be used to facilitate the reaction. A compound of Formula Y can have the structure shown herein wherein R^{3C} can be the same as R^{3A} as described with respect to a compound of Formula (Ia), each R^{c1} can be an optionally substituted C₁₋₄ alkyl; and PG^{6C} and PG^{7C} can each be a protecting group. In some embodiments, PG^{6C} can be a silyl ether group such as those described herein. In an embodiment, PG^{7C} can be a triarymethyl protecting group. Exemplary triarylmethyl protecting groups are described herein.

The phosphite of a compound of Formula Z can be oxidized to a phosphate. Suitable methods known to those skilled in the art and methods described herein can be used to perform the oxidation of the phosphite to a phosphate.

Using methods known to those skilled in the art, PG^{6C} can be removed from a compound of Formula AA to form a compound of Formula BB. As an example, if PG^{6C} is silyl ether protecting group, it can be removed using a tetra(alkyl)ammounium halide. In some embodiments, PG^{6C} can be selectively removed such that PG^{1C} is removed without removing one or more selected from PG^{2C}, PG^{3C}, PG^{4C}, PG^{5C} and PG^{7C}. For example, PG^{6C} can be removed using a reagent such as a tetra(alkyl)ammonium halide that does not remove PG^{2C}, PG^{3C}, PG^{4C}, PG^{5C} and/or PG^{7C}.

A compound of Formula CC can then be added to the 5′-OH of the 5′-terminal residue of a compound of Formula BB. In some embodiments, an activator can be used to promote the reaction. In an embodiment, each R^{c1} can be an optionally substituted C₁₋₄ alkyl; and each R^{7C}, each R^{8C} and each R^{9C} can be the same as R^{7B}, R^{8B} and R^{9B} as described herein with respect to a compound of Formula (Ia).

The protecting groups represented by PG^{2C}, PG^{3C}, PG^{4C}, PG^{5C} and PG^{7C} can be removed using methods known to those skilled in the art to form a compound of Formula (Ia). In some embodiments, protecting groups on the oxygens attached to the 2′ and 3′-positions of the 2-terminal residue represented by PG^{3C} and PG^{4C} can be removed selectively. For example, the protecting groups can be removed without removing any protecting groups selected from PG^{2C}, PG^{5C} and PG^{7C}. Alternatively, the protecting groups PG^{2C}, PG^{5C} and PG^{7C} can be selectively removed such that PG^{2C}, PG^{5C} and PG^{7C} can be removed without removing any protecting groups on the oxygens attached to the 2′ and 3′-positions such as PG^{3C} and PG^{4C}. In an embodiment, PG^{3C} and PG^{4C} can be removed before removing one or more selected from PG^{2C}, PG^{5C} and PG^{7C}. In another embodiment, PG^{3C} and PG^{4C} can be removed after removing one or more selected from PG^{2C}, PG^{5C} and PG^{7C}. In some embodiments, PG^{3C} and PG^{4C} can be removed almost simultaneously. In other embodiments, PG^{3C} and PG^{4C} can be removed sequentially. In some embodiments, PG^{2C}, PG^{5C} and PG^{7C} can be removed almost simultaneously. In other embodiments, PG^{2C}, PG^{5C} and PG^{7C} can be removed sequentially.

The methods of synthesis described above in Schemes 2a, 2b, 2c, 2d, 2e and 2f can be used to synthesize any of the compounds and any embodiments described herein such as those of Formulae (I) and/or (Ia).

### Pharmaceutical Compositions

An embodiment described herein relates to a pharmaceutical composition, that can include a therapeutically effective amount of one or more compounds described herein (e.g., a compound of Formula (I) and/or a compound of Formula (Ia)) and a pharmaceutically acceptable carrier, diluent, excipient or combination thereof.

The term “pharmaceutical composition” refers to a mixture of a compound disclosed herein with other chemical components, such as diluents or carriers. The pharmaceutical composition facilitates administration of the compound to an organism. Multiple techniques of administering a compound exist in the art including, but not limited to, oral, intramuscular, intraocular, intranasal, intravenous, injection, aerosol, parenteral, and topical administration. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like. Pharmaceutical compositions will generally be tailored to the specific intended route of administration.

The term “physiologically acceptable” defines a carrier, diluent or excipient that does not abrogate the biological activity and properties of the compound.

As used herein, a “carrier” refers to a compound that facilitates the incorporation of a compound into cells or tissues. For example, without limitation, dimethyl sulfoxide (DMSO) is a commonly utilized carrier that facilitates the uptake of many organic compounds into cells or tissues of a subject.

As used herein, a “diluent” refers to an ingredient in a pharmaceutical composition that lacks pharmacological activity but may be pharmaceutically necessary or desirable. For example, a diluent may be used to increase the bulk of a potent drug whose mass is too small for manufacture or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

As used herein, an “excipient” refers to an inert substance that is added to a pharmaceutical composition to provide, without limitation, bulk, consistency, stability, binding ability, lubrication, disintegrating ability etc., to the composition. A “diluent” is a type of excipient.

The pharmaceutical compositions described herein can be administered to a human patient per se, or in pharmaceutical compositions where they are mixed with other active ingredients, as in combination therapy, or carriers, diluents, excipients or combinations thereof. Proper formulation is dependent upon the route of administration chosen. Techniques for formulation and administration of the compounds described herein are known to those skilled in the art.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes. Additionally, the active ingredients are contained in an amount effective to achieve its intended purpose. Many of the compounds used in the pharmaceutical combinations disclosed herein may be provided as salts with pharmaceutically compatible counterions.

Suitable routes of administration may, for example, include oral, rectal, topical transmucosal, or intestinal administration; parenteral delivery, including intramuscular, subcutaneous, intravenous, intramedullary injections, as well as intrathecal, direct intraventricular, intraperitoneal, intranasal, intraocular injections or as an aerosol inhalant.

One may also administer the compound in a local rather than systemic manner, for example, via injection of the compound directly into the infected area, often in a depot or sustained release formulation. Furthermore, one may administer the compound in a targeted drug delivery system, for example, in a liposome coated with a tissue-specific antibody. The liposomes will be targeted to and taken up selectively by the organ.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions that can include a compound described herein formulated in a compatible pharmaceutical carrier may also be prepared, placed in an appropriate container, and labeled for treatment of an indicated condition.

### Methods of Use

One embodiment disclosed herein relates to a method of treating and/or ameliorating a disease or condition that can include administering to a subject a therapeutically effective amount of one or more compounds described herein, such as a compound of Formula (I) and/or a compound of Formula (Ia), or a pharmaceutical composition that includes a compound described herein.

Some embodiments disclosed herein relate to a method of ameliorating or treating a neoplastic disease that can include administering to a subject suffering from a neoplastic disease a therapeutically effective amount of one or more compounds described herein (e.g., a compound of Formula (I) and/or a compound of Formula (Ia)) or a pharmaceutical composition that includes one or more compounds described herein. In an embodiment, the neoplastic disease can be cancer. In some embodiments, the neoplastic disease can be a tumor such as a solid tumor. In an embodiment, the neoplastic disease can be leukemia. Exemplary leukemias include, but are not limited to, acute lymphoblastic leukemia (ALL), acute myeloid leukemia (AML) and juvenile myelomonocytic leukemia (JMML).

An embodiment disclosed herein relates to a method of inhibiting the growth of a tumor that can include administering to a subject having a tumor a therapeutically effective amount of one or more compounds described herein or a pharmaceutical composition that includes one or more compounds described herein.

Other embodiments disclosed herein relates to a method of ameliorating or treating a viral infection that can include administering to a subject suffering from a viral infection a therapeutically effective amount of one or more compounds described herein or a pharmaceutical composition that includes one or more compounds described herein. In an embodiment, the viral infection can be caused by a virus selected from an adenovirus, an Alphaviridae, an Arbovirus, an Astrovirus, a Bunyaviridae, a Coronaviridae, a Filoviridae, a Flaviviridae, a Hepadnaviridae, a Herpesviridae, an Alphaherpesvirinae, a Betaherpesvirinae, a Gammaherpesvirinae, a Norwalk Virus, an Astroviridae, a Caliciviridae, an Orthomyxoviridae, a Paramyxoviridae, a Paramyxoviruses, a Rubulavirus, a Morbillivirus, a Papovaviridae, a Parvoviridae, a Picornaviridae, an Aphthoviridae, a Cardioviridae, an Enteroviridae, a Coxsackie virus, a Polio Virus, a Rhinoviridae, a Phycodnaviridae, a Poxviridae, a Reoviridae, a Rotavirus, a Retroviridae, an A-Type Retrovirus, an Immunodeficiency Virus, a Leukemia Viruses, an Avian Sarcoma Viruses, a Rhabdoviruses, a Rubiviridae and/or a Togaviridae.

One embodiment disclosed herein relates to a method of ameliorating or treating a parasitic disease that can include administering to a subject suffering from a parasitic disease a therapeutically effective amount of one or more compounds described herein or a pharmaceutical composition that includes one or more compounds described herein. In an embodiment, the parasite disease can be Chagas' disease.

As used herein, a “subject” refers to an animal that is the object of treatment, observation or experiment. “Animal” includes cold- and warm-blooded vertebrates and invertebrates such as fish, shellfish, reptiles and, in particular, mammals. “Mammal” includes, without limitation, mice, rats, rabbits, guinea pigs, dogs, cats, sheep, goats, cows, horses, primates, such as monkeys, chimpanzees, and apes, and, in particular, humans.

As used herein, the terms “treating,” “treatment,” “therapeutic,” or “therapy” do not necessarily mean total cure or abolition of the disease or condition. Any alleviation of any undesired signs or symptoms of a disease or condition, to any extent can be considered treatment and/or therapy. Furthermore, treatment may include acts that may worsen the patient's overall feeling of well-being or appearance.

The term “therapeutically effective amount” is used to indicate an amount of an active compound, or pharmaceutical agent, that elicits the biological or medicinal response indicated. For example, a therapeutically effective amount of compound can be the amount need to prevent, alleviate or ameliorate symptoms of disease or prolong the survival of the subject being treated This response may occur in a tissue, system, animal or human and includes alleviation of the symptoms of the disease being treated. Determination of a therapeutically effective amount is well within the capability of those skilled in the art, especially in light of the detailed disclosure provided herein. The therapeutically effective amount of the compounds disclosed herein required as a dose will depend on the route of administration, the type of animal, including human, being treated, and the physical characteristics of the specific animal under consideration. The dose can be tailored to achieve a desired effect, but will depend on such factors as weight, diet, concurrent medication and other factors which those skilled in the medical arts will recognize.

As will be readily apparent to one skilled in the art, the useful in vivo dosage to be administered and the particular mode of administration will vary depending upon the age, weight, the severity of the affliction, and mammalian species treated, the particular compounds employed, and the specific use for which these compounds are employed. (See e.g., Fingl et al. 1975, in “The Pharmacological Basis of Therapeutics”, which is hereby incorporated herein by reference in its entirety, with particular reference to Ch. 1, p. 1). The determination of effective dosage levels, that is the dosage levels necessary to achieve the desired result, can be accomplished by one skilled in the art using routine pharmacological methods. Typically, human clinical applications of products are commenced at lower dosage levels, with dosage level being increased until the desired effect is achieved. Alternatively, acceptable in vitro studies can be used to establish useful doses and routes of administration of the compositions identified by the present methods using established pharmacological methods.

Although the exact dosage will be determined on a drug-by-drug basis, in most cases, some generalizations regarding the dosage can be made. The daily dosage regimen for an adult human patient may be, for example, an oral dose of between 0.01 mg and 3000 mg of each active ingredient, preferably between 1 mg and 700 mg, e.g. 5 to 200 mg. The dosage may be a single one or a series of two or more given in the course of one or more days, as is needed by the patient. In some embodiments, the compounds will be administered for a period of continuous therapy, for example for a week or more, or for months or years.

In instances where human dosages for compounds have been established for at least some condition, the present invention will use those same dosages, or dosages that are between about 0.1% and 500%, more preferably between about 25% and 250% of the established human dosage. Where no human dosage is established, as will be the case for newly-discovered pharmaceutical compositions, a suitable human dosage can be inferred from ED₅₀ or ID₅₀ values, or other appropriate values derived from in vitro or in vivo studies, as qualified by toxicity studies and efficacy studies in animals.

In cases of administration of a pharmaceutically acceptable salt, dosages may be calculated as the free base. As will be understood by those of skill in the art, in certain situations it may be necessary to administer the compounds disclosed herein in amounts that exceed, or even far exceed, the above-stated, preferred dosage range in order to effectively and aggressively treat particularly aggressive diseases or infections.

Dosage amount and interval may be adjusted individually to provide plasma levels of the active moiety which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compositions should be administered using a regimen which maintains plasma levels above the MEC for 10-90% of the time, preferably between 30-90% and most preferably between 50-90%. In cases of local administration or selective uptake, the effective local concentration of the drug may not be related to plasma concentration.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above may be used in veterinary medicine.

In non-human animal studies, applications of potential products are commenced at higher dosage levels, with dosage being decreased until the desired effect is no longer achieved or adverse side effects disappear. The dosage may range broadly, depending upon the desired effects and the therapeutic indication. Alternatively dosages may be based and calculated upon the surface area of the patient, as understood by those of skill in the art.

Compounds disclosed herein can be evaluated for efficacy and toxicity using known methods. For example, the toxicology of a particular compound, or of a subset of the compounds, sharing certain chemical moieties, may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, or monkeys, may be determined using known methods. The efficacy of a particular compound may be established using several recognized methods, such as in vitro methods, animal models, or human clinical trials. Recognized in vitro models exist for nearly every class of condition, including but not limited to cancer, cardiovascular disease, and various immune dysfunction. Similarly, acceptable animal models may be used to establish efficacy of chemicals to treat such conditions. When selecting a model to determine efficacy, the skilled artisan can be guided by the state of the art to choose an appropriate model, dose, and route of administration, and regime. Of course, human clinical trials can also be used to determine the efficacy of a compound in humans.

### EXAMPLES

Embodiments of the present invention are disclosed in further detail in the following examples, which are not in any way intended to limit the scope of the invention.

### Example 1

### Synthesis of Compounds 5 and 6

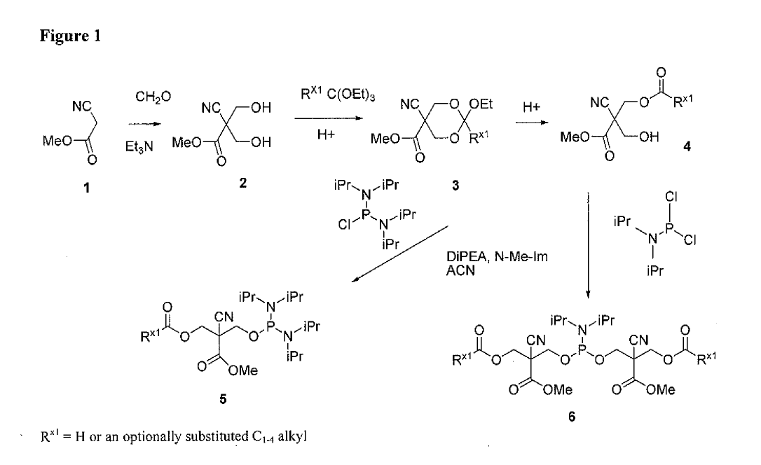

Compounds 5 and 6 are prepared according to the general scheme illustrated in FIG. 1 as follows:

Cyanoacetate 1 is bis-hydroxymethylated by treatment with formaldehyde in the presence of tertiary amine (e.g, Et₃N) to provide the bis-hydroxymethyl derivative 2. See Gizaev et al., Synthesis (1997), 1281-4, which is hereby incorporated by reference in its entirety. Acetal formation by treatment of diol 2 with orthoester R^{x1}C(OEt)₃ in acidic media (e.g., TFA/THF) leads to intermediate 3. Compound 3 is hydrolysed subsequently to alcohol 4 by treatment with TFA/H₂O/THF. The intermediate 4 is then converted to phosphoramidite 5 by standard phosphytilation with CIP(Ni(Pr)₂)₂ in the presence of DiPEA/N-Me-Im or into phosphoramidite 6 using Cl₂P(Ni(Pr)₂ as the phosphytilating reagent.

### Example 2

### Synthesis of Compound 7

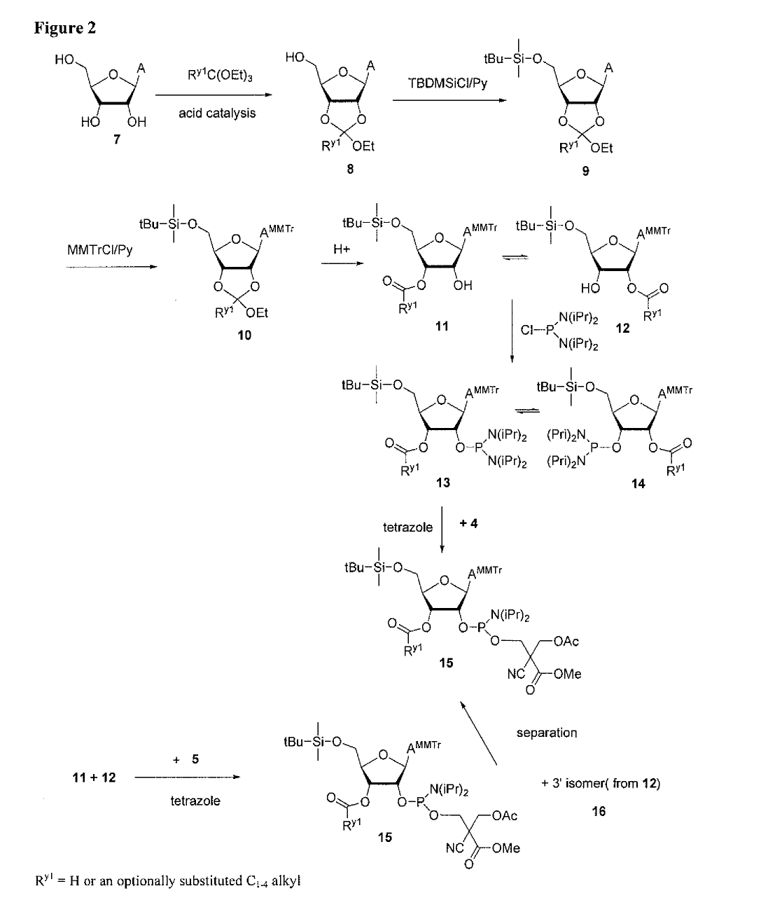

One synthetic route to form compound 15 is shown in the general scheme illustrated in FIG. 2.

Standard protection of the cis diol function in riboadenosine 7 using the procedure set forth in Griffin et al., Tetrahedron (1967), 23, 2301, which is hereby incorporated by reference in its entirety, leads to intermediate 8. Compound 8 is protected at 5′-OH by the introduction of a silyl protecting group (e.g., TBDMSiCl/Py), The N⁶ amino functional group is protected by a MMTr group which is introduced by treating nucleoside 9 with MMTrCl/Py. Mild acid treatment of nucleoside 10 results in hydrolysis of cyclic 2′,3′ ortho ester providing mixture of the protected nucleoside 11 with 2′ acyl isomer 12. If desired, compounds 11 and 12 can be separated by proceeding to the next step. Compound 11 and 12 are phosphytilated under standard conditions (e.g., using ClP(N(iPr)₂)₂ followed by in situ condensation with alcohol 4 in the presence of a condensation reagent (e,g., tetrazole or a derivative thereof). Compound 15 is obtained after separation from related 3′ isomer 16. Alternatively, a mixture of 2′ and 3′ acyl isomers 11 and 12 is subjected to condensation with reagent 5 in presence of tetrazole or a derivative thereof. If desired, the resulting phosphoramidites 15 and 16 can be separated.

### Example 3

### Synthesis of Compound 25 and 26

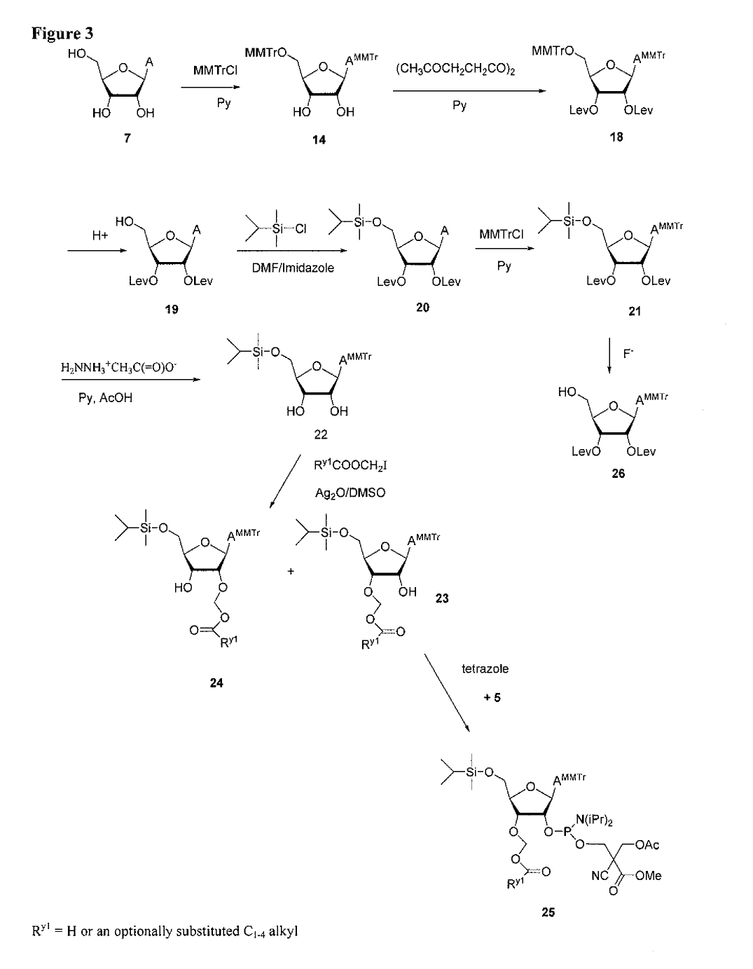

One synthetic route to form compounds 25 and 26 is shown in the general scheme illustrated in FIG. 3.

Selective protection of primary 5′-OH and N⁶-aminno group in riboadenosine by treatmeant with MMTrCl/Py followed by introduction of levulinyl protecting groups at 2′ and 3′ OH (e.g., using Lev₂O/Py) leads to fully protected nucleoside 18. Removal of acid labile MMTr groups from 18 and selective protection of 5′-OH by silyl protecting group (e.g, using iPrSiCl/DMF/omidazole) leads to intermediate 20. MMTr can be selectively added at N⁶ amino group (e.g, using MMTrCl/py) of compound 20 to form compound 21. Removal of 5′ silyl group from intermediate 21 provides 2′ terminal building block 26 whereas removal of 2′,3′cis diol protecting levulinyl groups (e.g., using H₂NNH₃-acetate/Py/AcOH) from the same compound provides nucleoside 22. See Jeker et al., Helv. Chim. Acta. (1988), 71, 1895, which is hereby incorporated by reference in its entirety. Acyloxymethyl group can be added to compound 22 by alkylation with R^{y1}COOCH₂X, wherein X is a leaving group (e.g., generated in situ from relative Cl derivative in presence of NaI) in presence of Ag₂O in DMSO. Separation of 2′ and 3′ isomers 24 and 25 followed by phosphytilation with reagent 5 in presence of tetrazole forms compound 25.

### Example 4

### Synthesis of 3′-O Acyl Trimer and 3′-O Acyloxymethyl Trimer

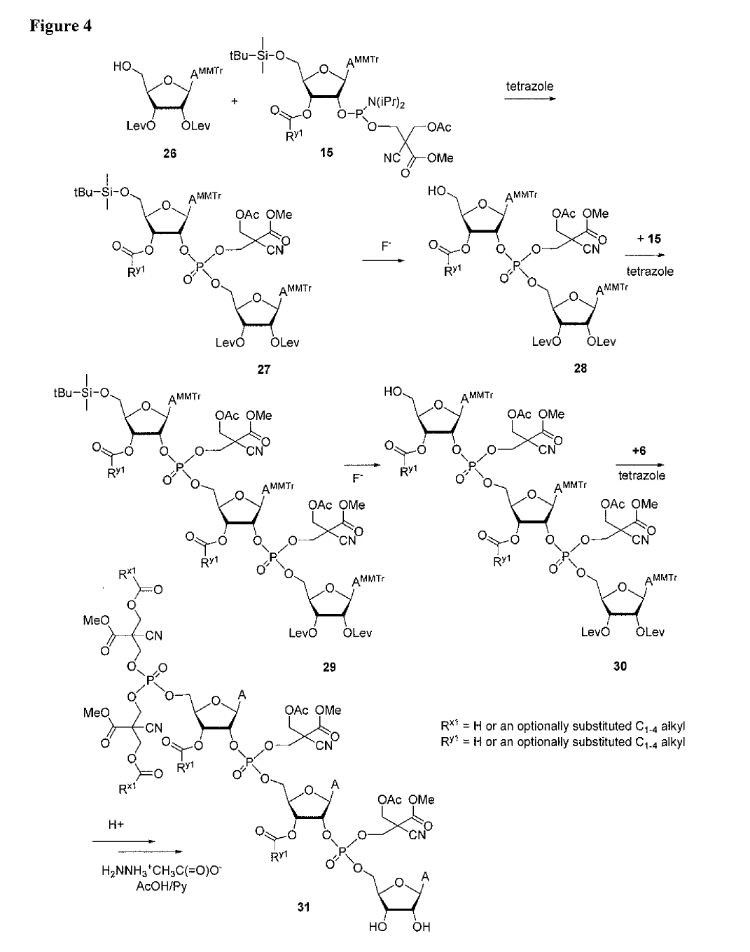

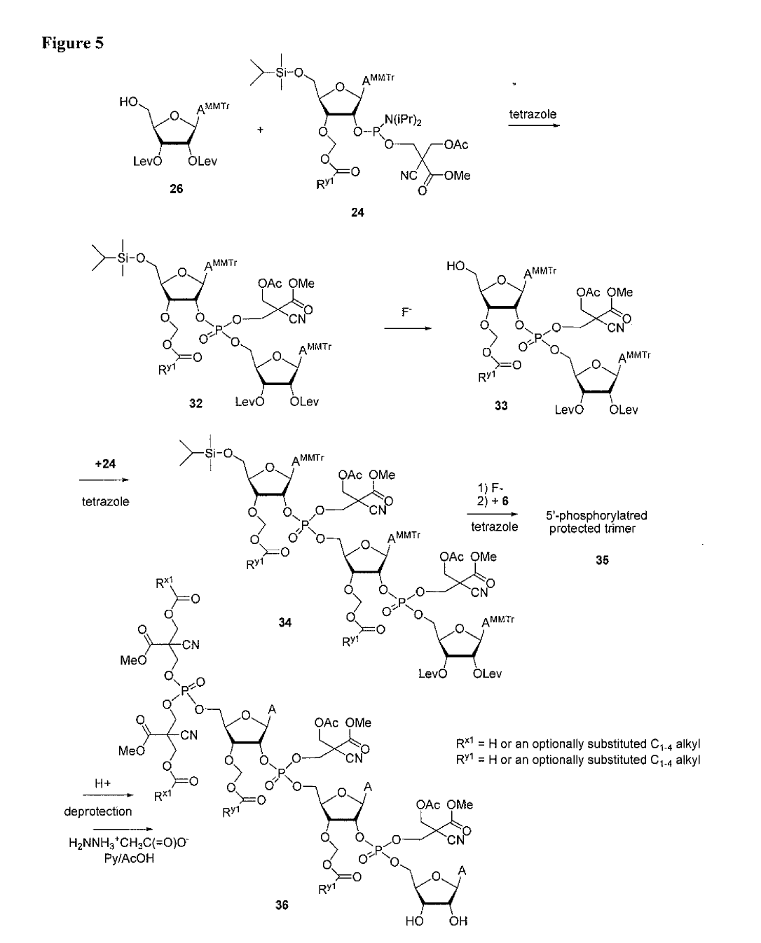

Exemplary synthetic routes to form trimers 31 and 36 are shown in the general scheme illustrated in FIGS. 4 and 5.

Compound 26 is condensed with phosphoramidite 15 in presence of tetrazole or a derivative thereof (e.g., S-Et or Bzl) to form the protected dimer 27. Removal of 5′ protecting silyl group on 27 leads to the formation of 5′-deprotected dimer 28 which undergoes another coupling with phosphoramidite 15 to form protected trimer 29. Removal of the 5′-silyl group from compound 29 provides 5′-deprotected intermediate 30 which is then coupled with phosphoramidite 6 in the presence of tetrazole or a derivative thereof. The N⁶ position of the adenosine residues are deprotected by acid treatment, The levulinyl groups at the 2′- and 3′-OH of the terminal 2′-adenosine moiety are also removed using for example H₂NNH₃-acetate/Py/AcOH. Final purification gives trimer 31 with protected phosphate functions and 3′-O-Acyl groups.

The 3′-O-Aacyloxymethyl trimer, compound 36, is assembled starting with compound 26 which is coupled in the presence of tetrazole or a derivative thereof (e.g., S-Et or Bzl) with phosphoramidite 24 to produce the protected dimer 32. The 5′-OH on dimer 24 is deprotected by removal of the silyl protecting group with F⁻ treatment. The 5′ deprotected dimer 33 is isolated and coupled again with phosphoramidite 24 resulting in the protected trimer, compound 34. The 5′ deprotection of trimer 34 by F³¹ treatment followed by coupling with phosphoramidite 6 results in 5′-phosphorylated protected trimer, compound 35. The N⁶ position of the adenosine residues, and 2′- and 3′-OH of the terminal 2′-adenosine moiety are deprotected as described above. Final purification provides compound 36 having protected phosphate functionalities and 3′-O-acyloxymethyl groups.

### Example 5

### Synthesis of Modified Trimers

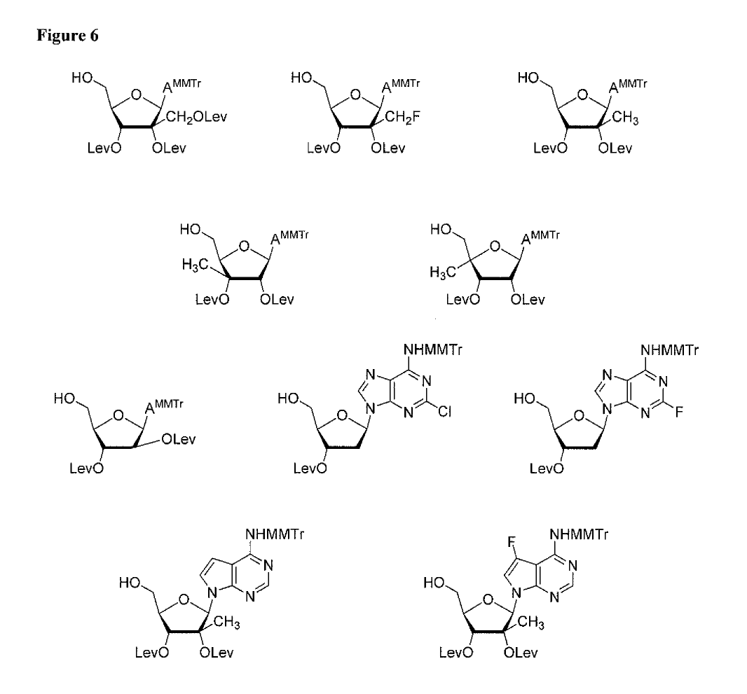

It is worth noting that the schemes shown in FIGS. 1-5 are universal and can be used for introduction of a modified nucleoside (e.g., an anti-viral, anti-neoplastic and/or anti-parasitic). Exemplary starting modified nucleosides are shown in FIG. 6. Preferably, the modified nucleoside analog has a 5′-OH.

### Example 6

### 1-Methyl 3-Acetoxy-2-Cyano-2-(Hydroxymethyl)Propanoate

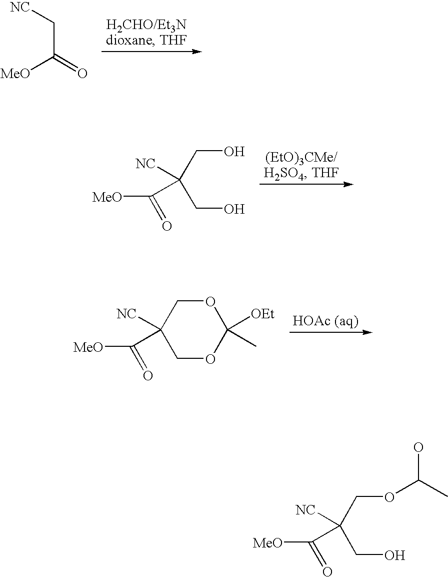

### Example 7

### 2-Cyano-3-(Ethylamino)-2-(Hydroxymethyl)-3-Oxopropyl Acetate

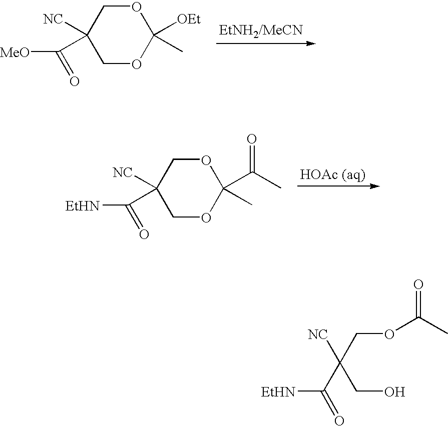

### Example 8

### Kinetic Studies

Preparation of the cell extract, 10×10⁶ of human prostate carcinoma cells (PC3) were treated with 10 mL of RIPA-buffer [15 mM Tris-HCl pH 7.5, 120 mM NaCl, 25 mM KCl, 2 mM EDTA, 2 mM EGTA, 0,1% Deoxycholic acid, 0,5% Triton X-100, 0,5% PMSF supplemented with Complete Protease Inhibitor Cocktail (Roche Diagnostics GmBH, Germany)] at 0° C. for 10 min. Most of the cells were disrupted by this hypotonic treatment and the remaining ones were disrupted mechanically. The cell extract obtained was centrifuged (900 rpm, 10 min) and the pellet was discarded. The extract was stored at −20° C.

Stability of Test Compounds in the cell extract. The cell extract was prepared as described above (1 mL), and was diluted with a 9-fold volume of HEPES buffer (0.02 mol L⁻¹, pH 7.5, I=0.1 mol L⁻¹ with NaCl). A test compound (0.1 mg) was added into 3 mL of this HEPES buffered cell extract and the mixture was kept at 22±1° C. Aliquots of 150 μL were withdrawn at appropriate intervals, filtered with SPARTAN 13A (0.2 μm) and cooled in an ice bath. The aliquots were analyzed immediately by HPLC-ESI mass spectroscopy (Hypersil RP 18, 4.6×20 cm, 5 μm). For the first 10 min, 0.1% aq formic acid containing 4% MeCN was used for elution and then the MeCN content was increased to 50% by a linear gradient during 40 min.

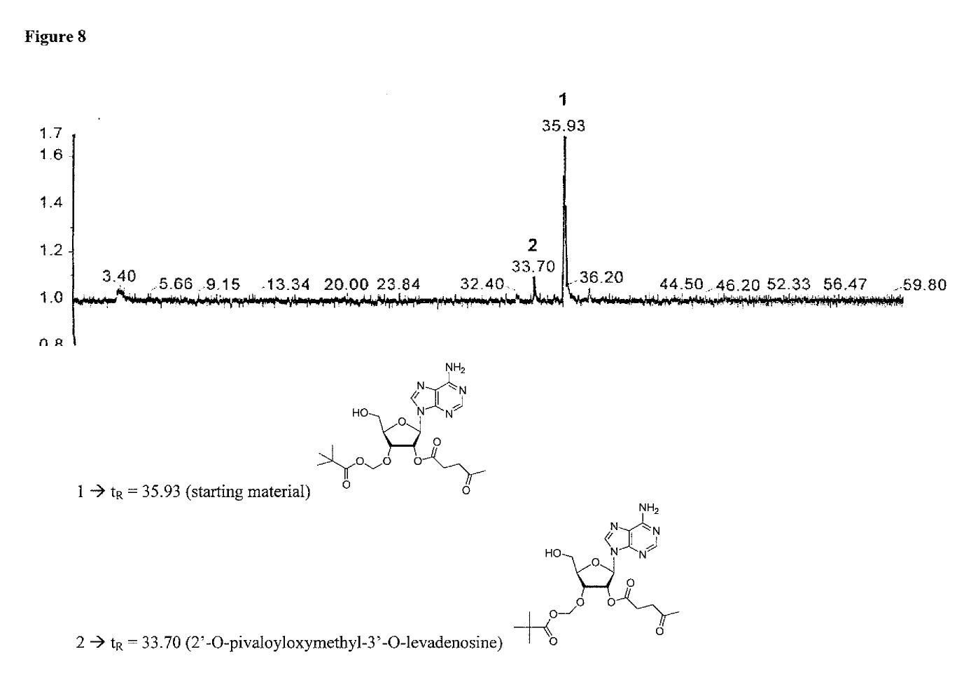

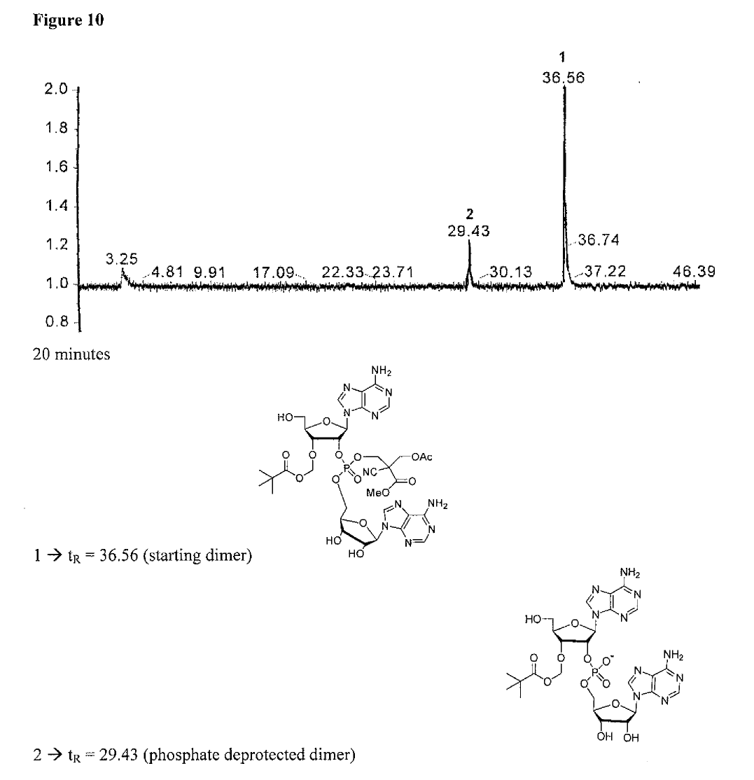

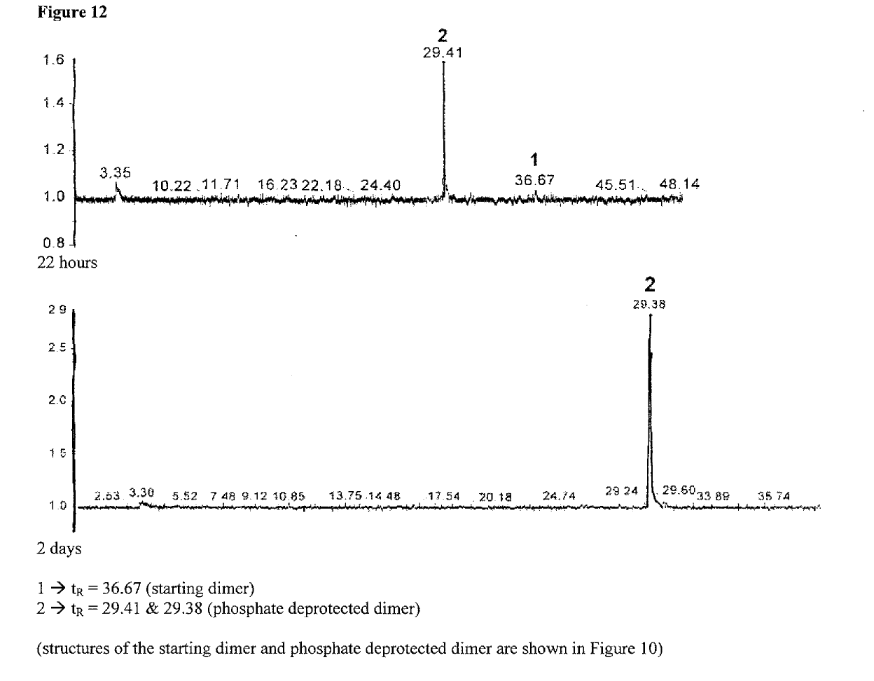

The results of the stability tests in cell extract are shown in FIGS. 8-13. FIG. 8 show a plot of a 3′O-acyloxymethyl protected mono-nucleoside after 10 minutes in cell extract diluted with HEPES buffer.

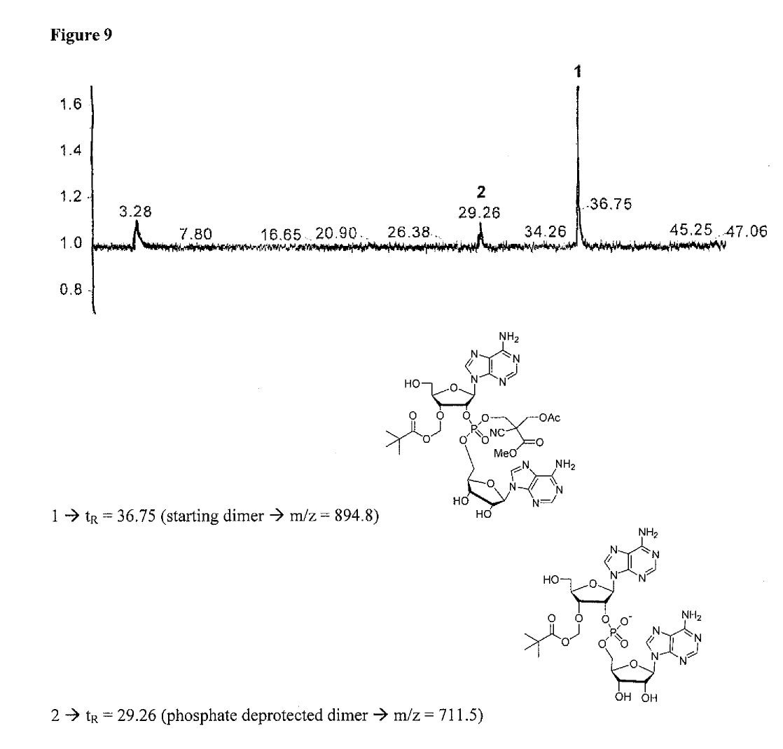

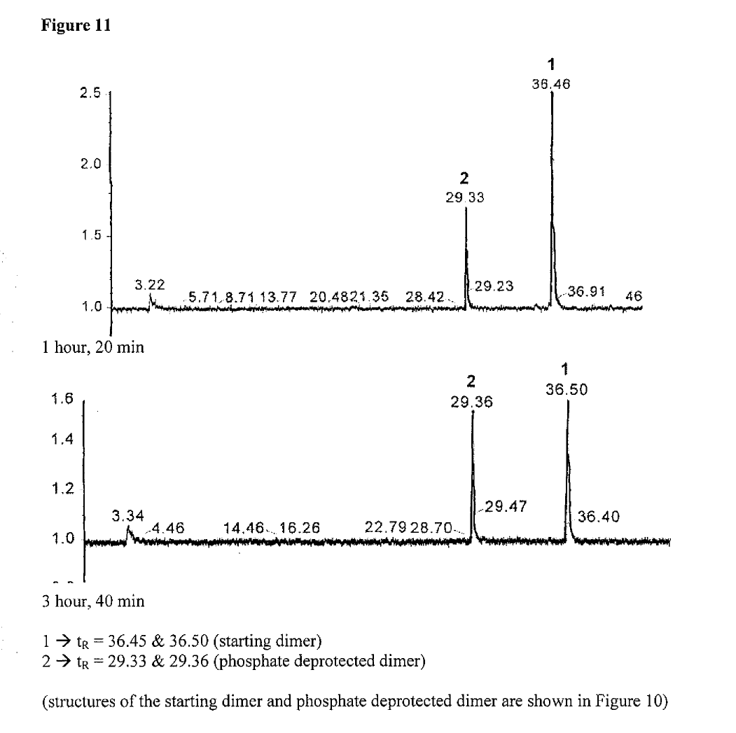

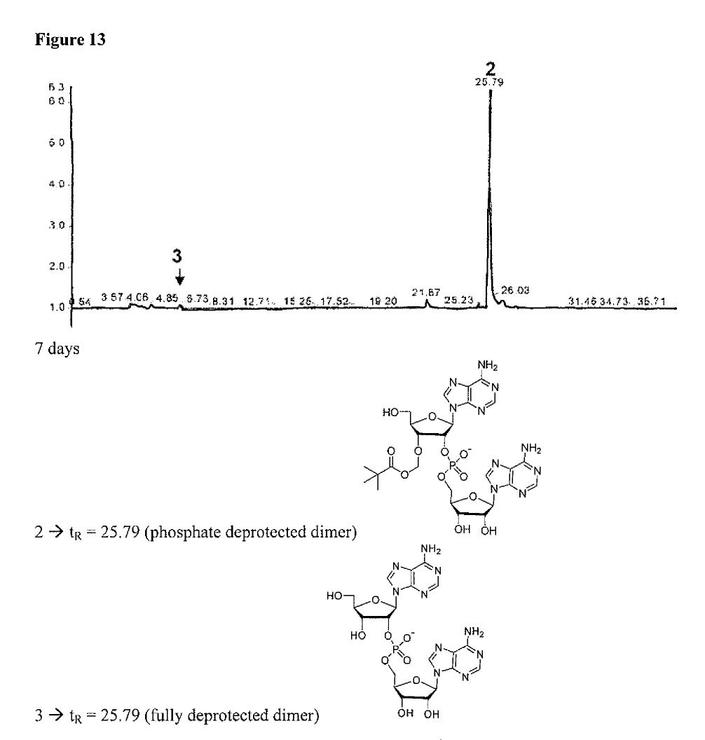

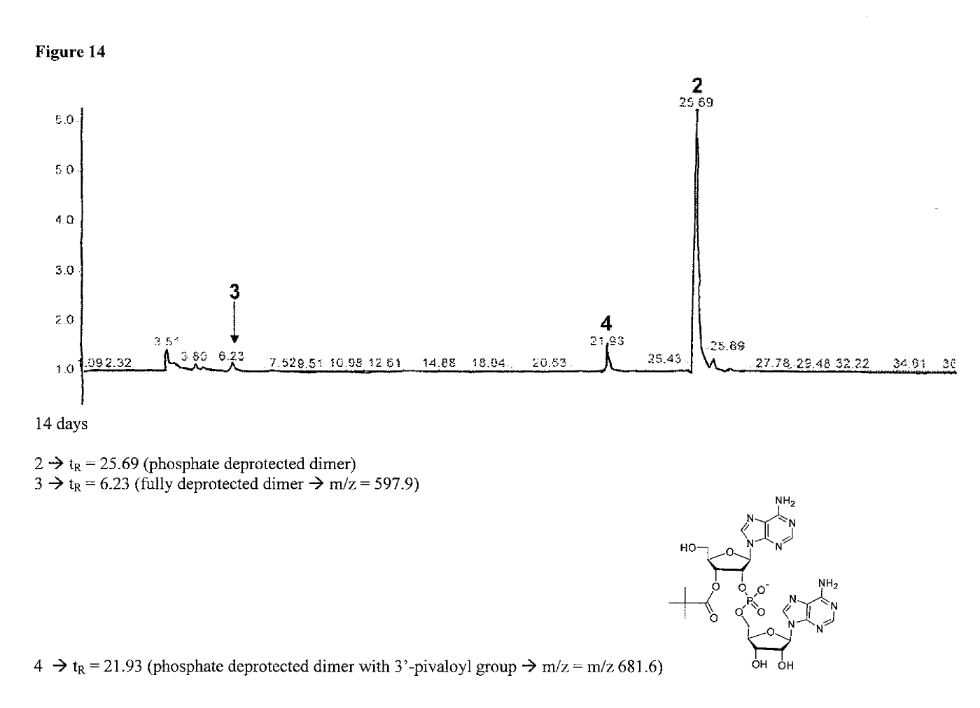

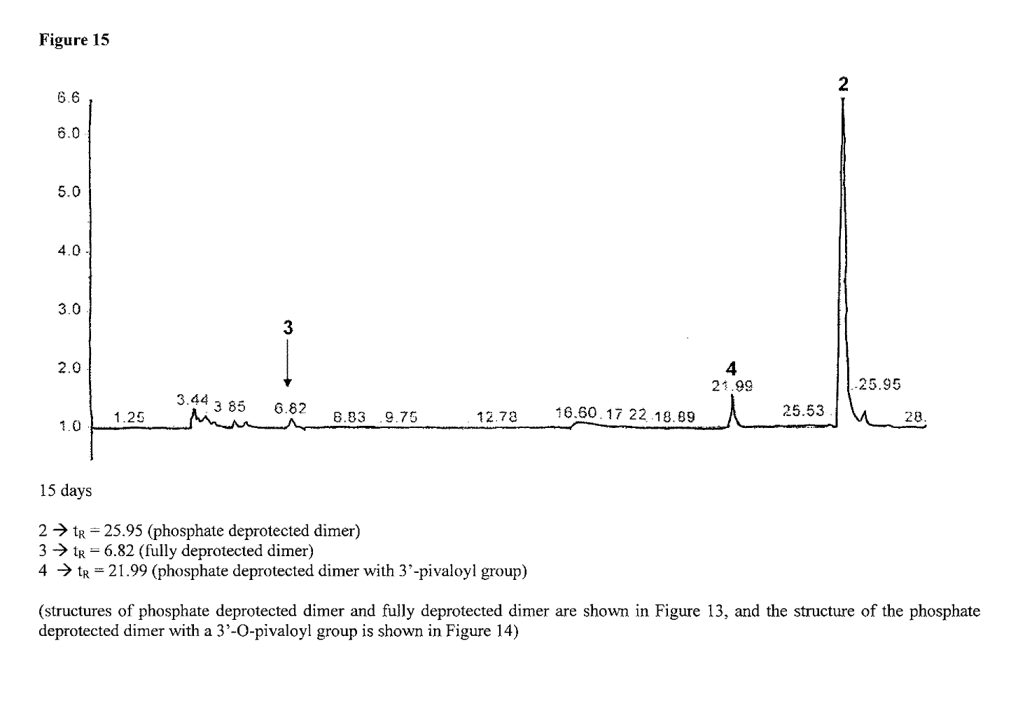

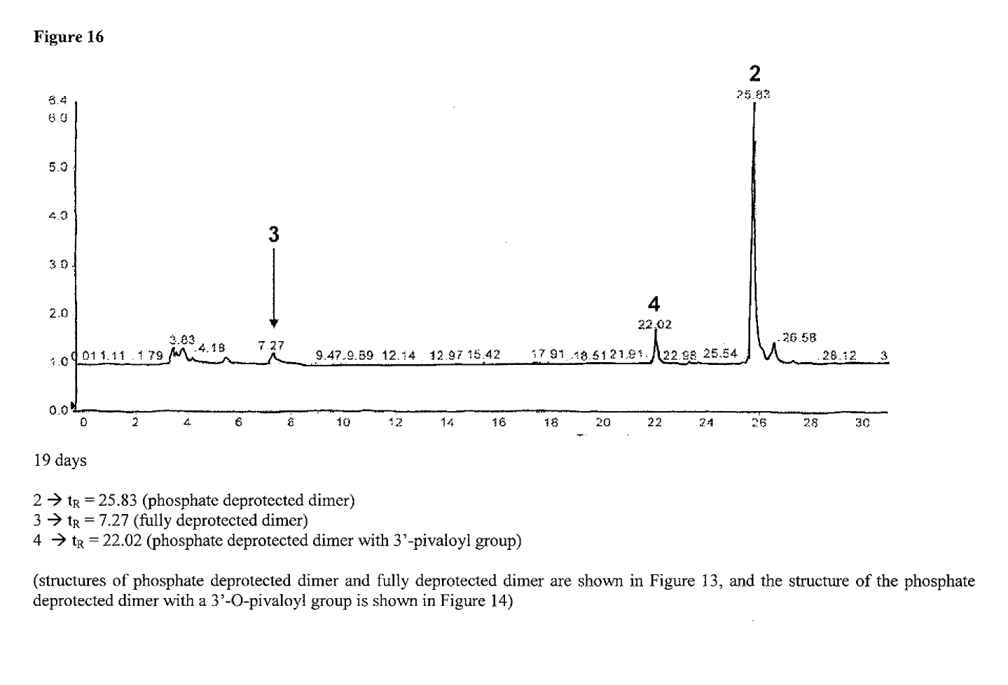

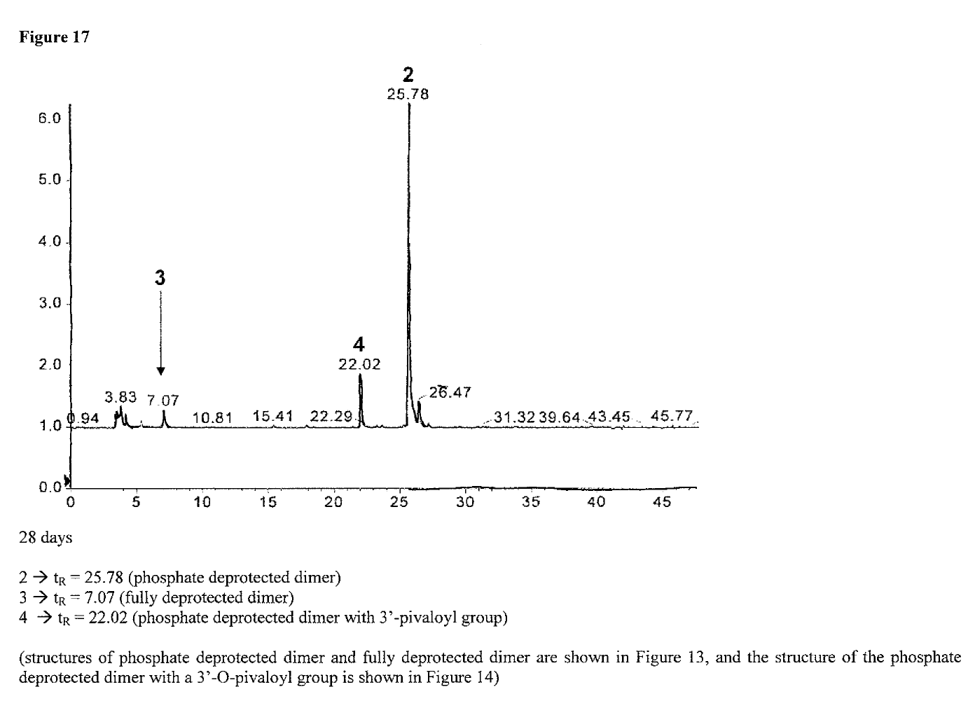

FIGS. 9-13 show plots of a 3′O-acyloxymethyl and phosphate protected dimer at time zero, 20 minutes, 1 hour and 20 minutes, 3 hours and 40 minutes, 2 days and 7 days in cell extract diluted with HEPES buffer. As shown in FIG. 11, the 2,2-disubstititued-3-acyloxypropyl protecting group was readily removed from the dimer. After almost a day, the starting dimer was completely converted to the deprotected phosphate dimer. The deprotected phosphate dimer then slowly converted to the fully deprotected dimer. See FIG. 13. Additional cell extract was then added to a concentration of (3 mL:10 mL cell extract:volume of solution). FIGS. 14-17 show plots of a 3′O-acyloxymethyl and phosphate protected dimer at 14 days, 15 days, 19 days and 28 days. As shown by FIGS. 14-17, the deprotected phosphate dimer continued to be converted to the fully deprotected dimer.

Stability of Test Compounds towards Porcine Liver Esterase. A test compound (1 mg) and 3 mg (48 units) of Sigma Porcine Liver Esterase (66H7075) were dissolved in 3 mL of HEPES buffer (0.02 mol L⁻¹, pH 7.5, I=0.1 mol L⁻¹ with NaCl). The stability test was carried out as described above for the cell extract.

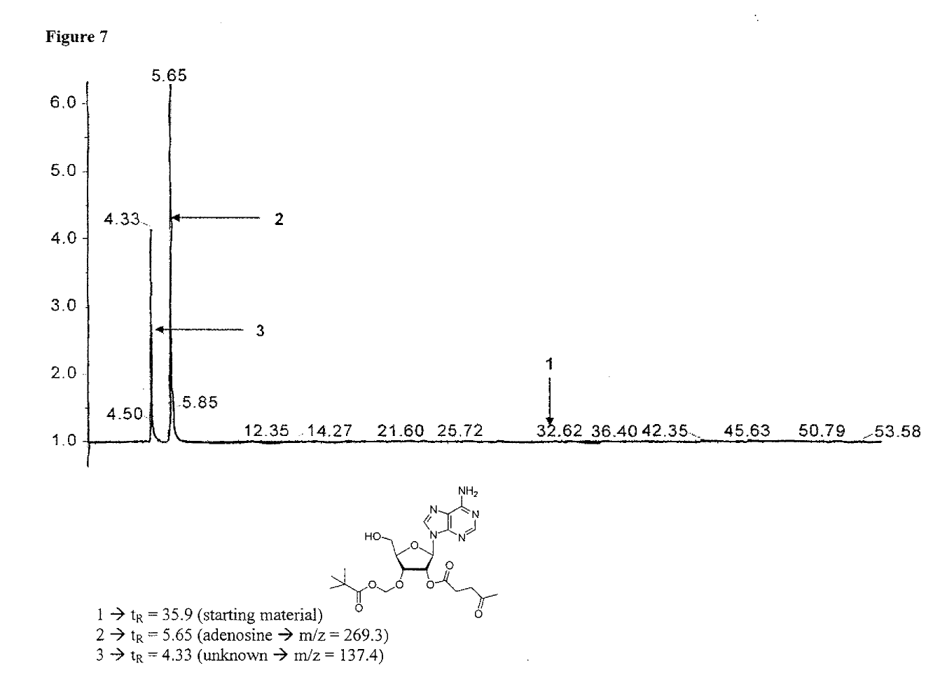

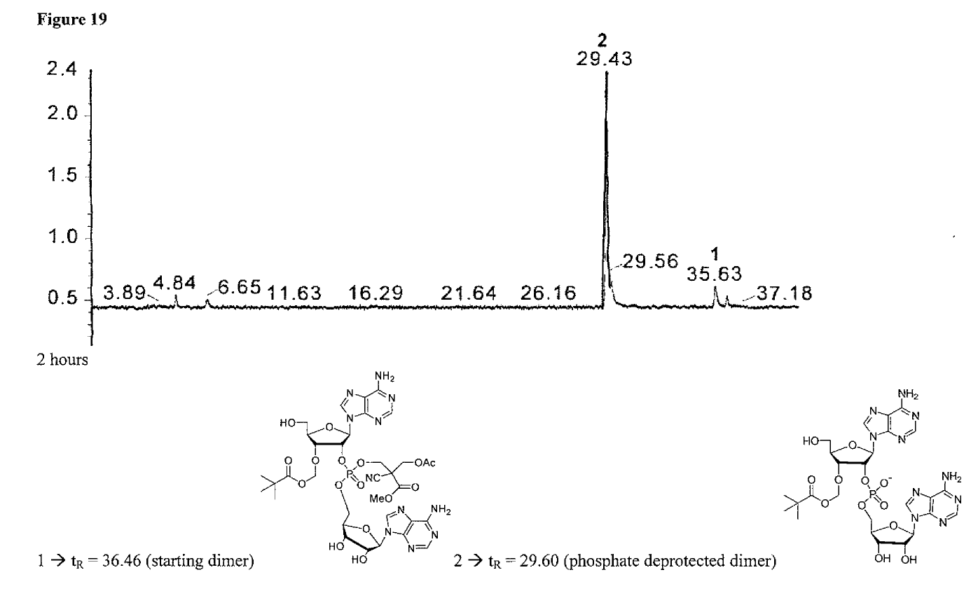

The results of the stability tests after exposures to porcine liver esterase (PLE) are shown in FIGS. 7 and 18-20. FIG. 7 shows a plot of a 3′O-acyloxymethyl protected mono-nucleoside after 5 days of exposure to PLE. As shown by FIG. 7, the PLE completely removed the 3′-O-acyloxymethyl group from the mono-nucleoside.

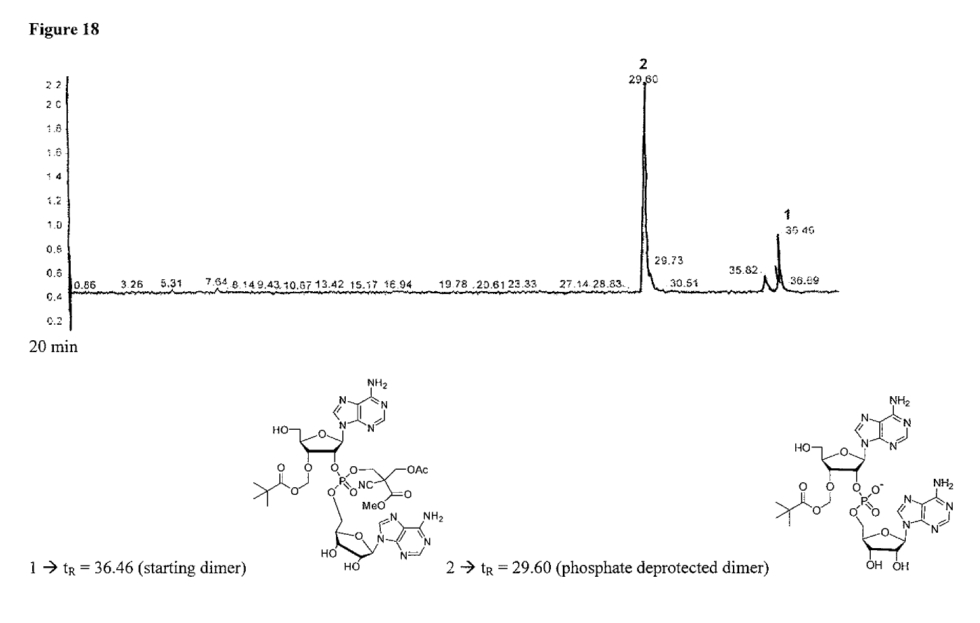

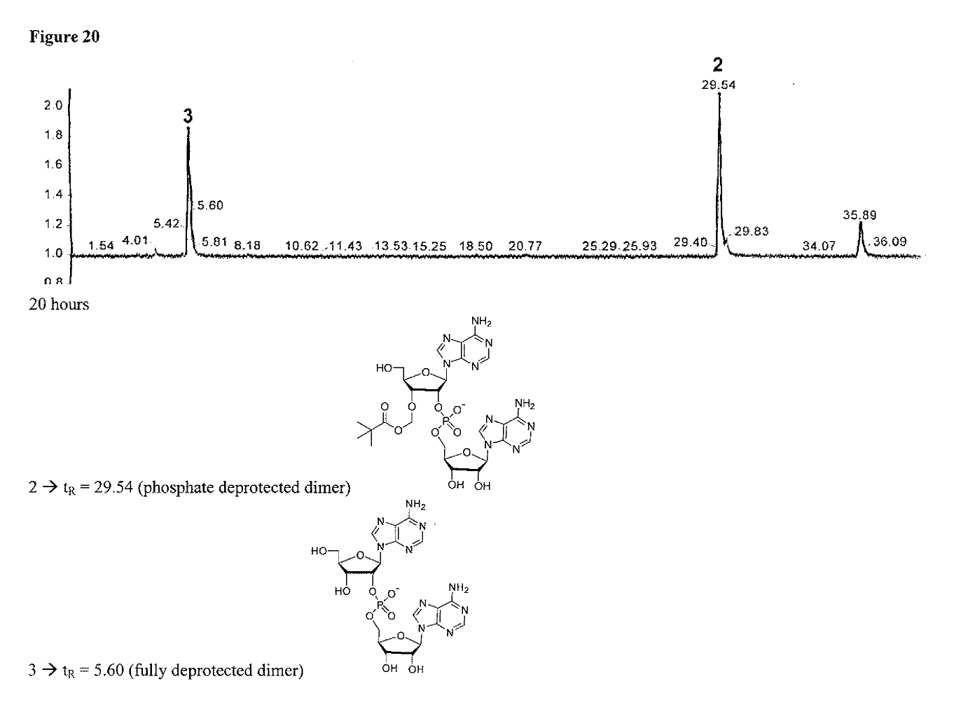

FIGS. 18-20 shows plots of a 3′O-acyloxymethyl and phosphate protected dimer after 20 minutes, 2 hours, and 20 hours of exposure PLE, respectively. The PLE easily removed the phosphate 2,2-disubstititued-3-acyloxypropyl protecting group from the dimer, as shown in FIG. 18. By comparison, the 3′-O-acyloxymethyl group on the dimer was removed by the PLE at a much slower rate. However, after about 20 hours, most of the starting dimer had been transformed to either the phosphate deprotected or fully deprotected dimer, as shown in FIG. 20.

Stability tests in human serum. Stability tests in human serum are carried out as described for the whole cell extract. The measurements are carried out in serum diluted 1:1 with HEPES buffer (0.02 mol L⁻¹, pH 7.5, I=0.1 mol L⁻¹ with NaCl).

It will be understood by those of skill in the art that numerous and various modification can be made without departing from the spirit of the present invention. Therefore, it should be clearly understood that the forms of the present invention are illustrative only and not intended to limit the scope of the present invention.

What is claimed is:

1. A compound of Formula (I), or a pharmaceutically acceptable salt, prodrug or prodrug ester thereof: 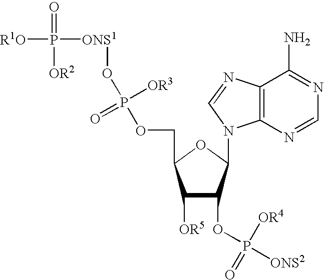
wherein:
each R¹, R², R³ and R⁴ are each independently absent, hydrogen or 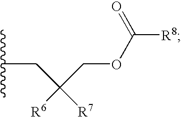
each R⁵ are each independently selected from the group consisting of hydrogen, —C(═O)R⁹, and —C(R¹⁰)₂—O—C(═O)R¹¹;
each R⁶ and each R⁷ are each independently selected from the group consisting of —C≡N, an optionally substituted 1-oxoalkyl, an optionally substituted alkoxycarbonyl and an optionally substituted alkylaminocarbonyl;
each R⁸, each R⁹, each R¹⁰ and each R¹¹ are each hydrogen or an optionally substituted C₁₋₄-alkyl;
NS¹ and NS² are independently selected from the group consisting of a nucleoside, a protected nucleoside, a nucleoside derivative and a protected nucleoside derivative.

2. The compound of claim 1, wherein R⁶ is —C≡N.

3. The compound of claim 2, wherein R⁷ is selected from the group consisting of an optionally substituted alkoxycarbonyl, an optionally substituted alkylaminocarbonyl and an optionally substituted 1-oxoalkyl.

4. The compound of claim 3, wherein the optionally substituted C₁₋₄ alkoxycarbonyl is —C(═O)OCH₃.

5. The compound of claim 3, wherein the optionally substituted C₁₋₄ alkylaminocarbonyl is —C(═O)NHCH₂CH₃.

6. The compound of claim 3, wherein the optionally substituted 1-oxoalkyl is —C(═O)CH₃.

7. The compound claim 3, wherein R⁸ is an optionally substituted C₁₋₄-alkyl.

8. The compound of claim 7, wherein each 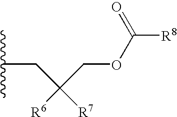
is independently

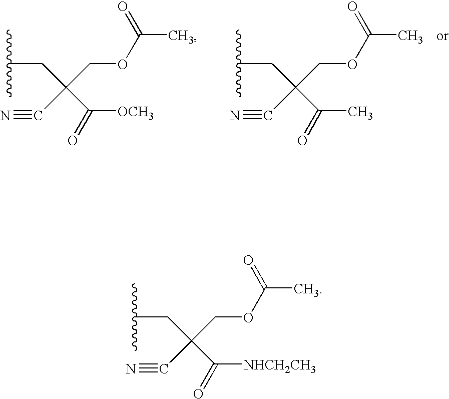

9. The compound of claim 1, wherein R⁵ is —C(═O)R⁹.

10. The compound of claim 9, wherein R⁹ is unsubstituted or substituted C₁₋₄-alkyl.

11. The compound of claim 1, wherein R⁵ is —C(R¹⁰)₂—O—C(═O)R¹¹.

12. The compound of claim 11, wherein each R¹⁰ is hydrogen and R¹¹ is unsubstituted or substituted C₁₋₄-alkyl.

13. The compound of claim 12, wherein R¹¹ is methyl or tert-butyl.

14. The compound of claim 1, wherein NS¹ is 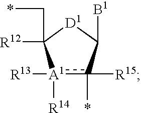
wherein:
A¹ is selected from the group consisting of C, O and S;
B¹ is an optionally substituted heterocyclic base or a derivative thereof;
D¹ is C═CH₂ or O;
R¹² is selected from the group consisting of hydrogen, azido, —CN, an optionally substituted C₁₋₄ alkyl and an optionally substituted C₁₋₄ alkoxy;
R¹³ is absent or selected from the group consisting of hydrogen, halogen, hydroxy and an optionally substituted C₁₋₄ alkyl;
R¹⁴ is absent or selected from the group consisting of hydrogen, halogen, azido, amino, hydroxy, —OC(═O)R¹⁶, and —OC(R¹⁷)₂—O—C(═O)R¹⁸;
R¹⁵ is selected from the group consisting of hydrogen, halogen, hydroxy, —CN, —NC, an optionally substituted C₁₋₄ alkyl, an optionally substituted haloalkyl and an optionally substituted hydroxyalkyl;
each R¹⁶, each R¹⁷ and each R¹⁸ are independently hydrogen or an optionally substituted C₁₋₄-alkyl; and
* represents a point of attachment.

15. The compound of claim 14, wherein R¹⁴ is —OC(═O)R¹⁶.

16. The compound of claim 15, wherein R¹⁶ is unsubstituted or substituted C₁₋₄-alkyl.

17. The compound of claim 14, wherein R¹⁴ is —OC(R¹⁷)₂—O—C(═O)R¹⁸.

18. The compound of claim 17, wherein each R¹⁷ is hydrogen and R¹⁸ is unsubstituted or substituted C₁₋₄-alkyl.

19. The compound of claim 14, wherein B¹ is selected from the group consisting of: 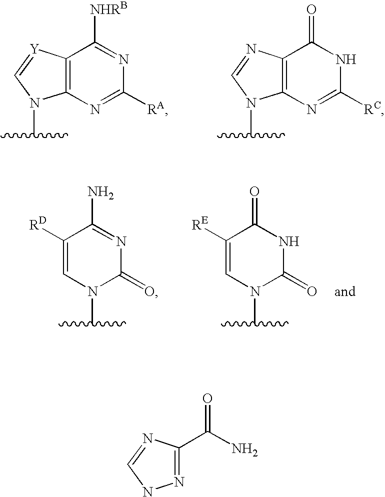
wherein:
R^{A} is hydrogen or halogen;
R^{B} is hydrogen, an optionally substituted C₁₋₄alkyl, or an optionally substituted C₃₋₈ cycloalkyl;
R^{C} is hydrogen or amino;
R^{D} is hydrogen or halogen;
R^{E} is hydrogen or an optionally substituted C₁₋₄alkyl; and
Y is N or CR^{F}, wherein R^{F} hydrogen, halogen or an optionally substituted C₁₋₄-alkyl.

20. The compound of claim 1, wherein NS¹ is selected from the group consisting of: 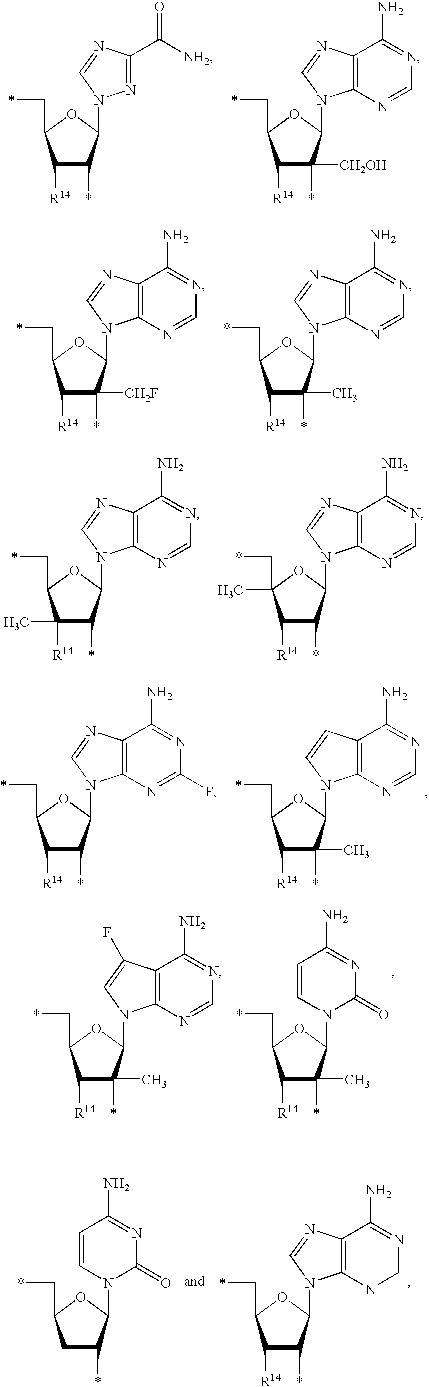
wherein:
R¹⁴ is absent or selected from the group consisting of hydrogen, halogen, azido, amino, hydroxy, —OC(═O)R¹⁶, and —OC(R¹⁷)₂—O—C(═O)R¹⁸, wherein each R¹⁶, each R¹⁷ and each R¹⁸ are independently hydrogen or an optionally substituted C₁₋₄-alkyl; and
* represents a point of attachment.

21. The compound of claim 1, wherein NS¹ is selected from the group consisting of anti-neoplastic agent, an anti-viral agent and an anti-parasitic agent.

22. The compound of claim 1, wherein NS² has the structure: 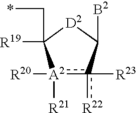
wherein:
A² is selected from the group consisting of C, O and S;
B² is an optionally substituted heterocyclic base or a derivative thereof;
D² is C═CH₂ or O;
R¹⁹ is selected from the group consisting of hydrogen, azido, —CN, an optionally substituted C₁₋₄ alkyl and an optionally substituted C₁₋₄ alkoxy;
R²⁰ is absent or selected from the group consisting of hydrogen, halogen, hydroxy and an optionally substituted C₁₋₄ alkyl;
R²¹ is absent or selected from the group consisting of hydrogen, halogen, azido, amino and hydroxy;
R²² is selected from the group consisting of hydrogen, halogen, hydroxy, —CN, —NC, an optionally substituted C₁₋₄ alkyl and an optionally substituted C₁₋₄ alkoxy;
R²³ is selected from the group consisting of hydrogen, halogen, hydroxy, —CN, —NC, an optionally substituted C₁₋₄ alkyl, an optionally substituted haloalkyl and an optionally substituted hydroxyalkyl, or when the bond to R²² indicated by [symbol] is a double bond, then R²² and R²³ can be taken together to form a C₁₋₄ alkenyl; and
* represents a point of attachment.

23. The compound of claims 22, wherein B″ is selected from the group consisting of: 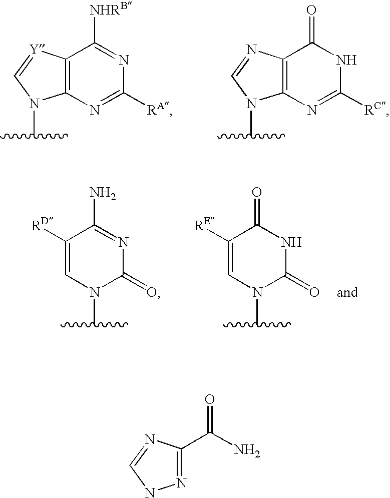
wherein:
R^{A″} is hydrogen or halogen;
R^{B″} is hydrogen, an optionally substituted C₁₋₄alkyl, or an optionally substituted C₃₋₈ cycloalkyl;
R^{C″} is hydrogen or amino;
R^{D″} is hydrogen or halogen;
R^{E″} is hydrogen or an optionally substituted C₁₋₄alkyl; and
Y is N or CR^{F″}, wherein R^{F″} hydrogen, halogen or an optionally substituted C₁₋₄-alkyl.

24. The compound of claim 1, wherein NS² is selected from the group consisting of: 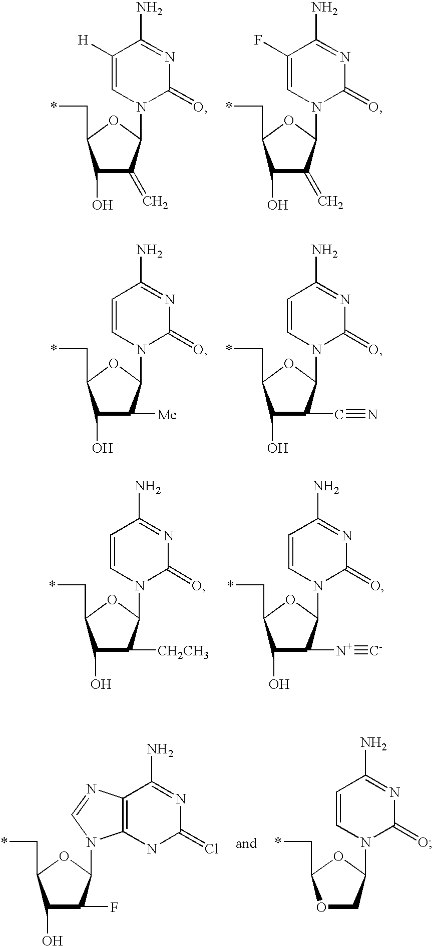
wherein * represents a point of attachment.

25. The compound of claim 1, wherein NS² is selected from the group consisting of: 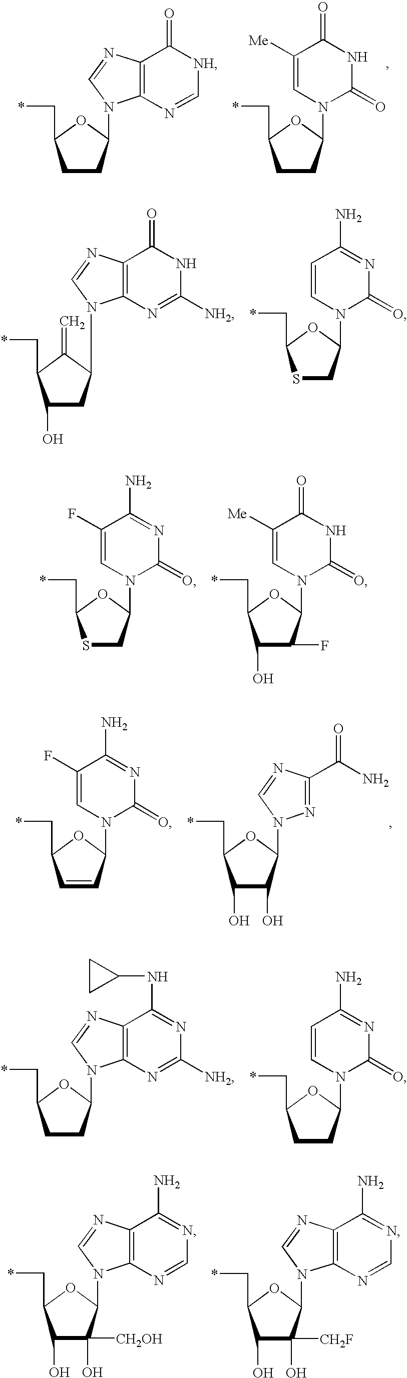 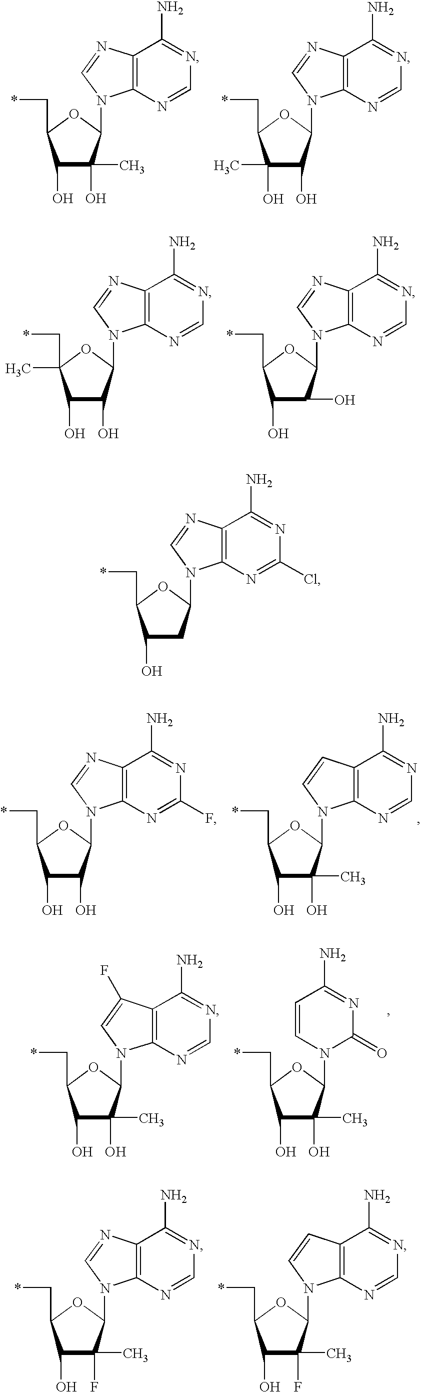 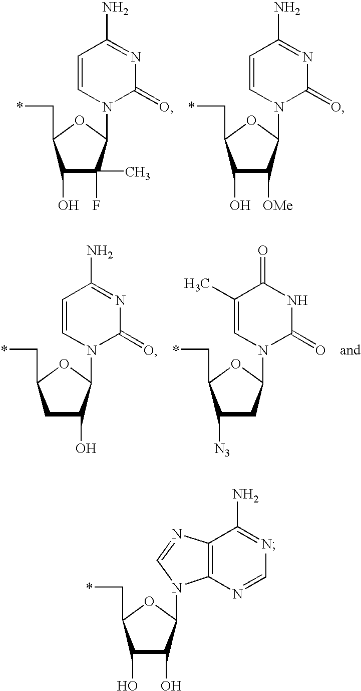
wherein * represents a point of attachment.

26. The compound of claim 1, wherein NS² is selected from the group consisting of anti-neoplastic agent, an anti-viral agent and an anti-parasitic agent.

27. A compound of Formula (Ia), or a pharmaceutically acceptable salt, prodrug or prodrug ester thereof: 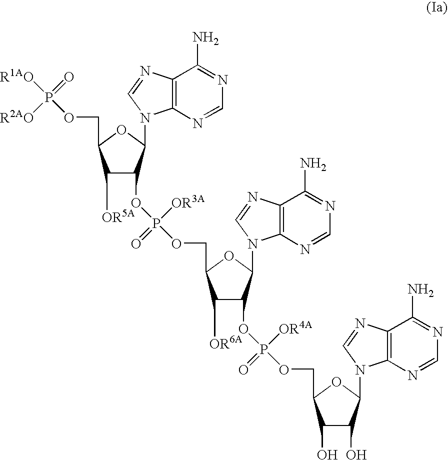
wherein:
R^{1A}, R^{2A}, R^{3A} and R^{4A} are each 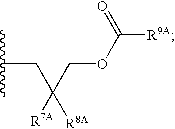
R^{5A} and R^{6A} are independently selected from the group consisting of hydrogen, —C(═O)R^{16A}, and —C(R^{11A})₂—O—C(═O)R^{12A};
each R^{7A} and each R^{8A} are each independently selected from the group consisting of —C≡N, an optionally substituted 1-oxoalkyl, an optionally substituted alkoxycarbonyl and an optionally substituted alkylaminocarbonyl;
each R^{9A}, each R^{10A}, each R^{11A} and each R^{12A} are each hydrogen or an optionally substituted C₁₋₄-alkyl;
wherein R^{1A}, R^{2A}, R^{3A} and R^{4A} can be the same or different from each other.

28. The compound of claim 27, wherein R^{7A} is —C≡N.

29. The compound of claim 28, wherein R^{8A} is selected from the group consisting of an optionally substituted alkoxycarbonyl, an optionally substituted alkylaminocarbonyl and an optionally substituted 1-oxoalkyl.

30. The compound of claim 29, wherein the optionally substituted C₁₋₄ alkoxycarbonyl is —C(═O)OCH₃.

31. The compound of claim 29, wherein the optionally substituted C₁₋₄ alkylaminocarbonyl is —C(═O)NHCH₂CH₃.

32. The compound of claim 29, wherein the optionally substituted 1-oxoalkyl is —C(═O)OCH₃.

33. The compound of claim 29, wherein R^{9A} is an optionally substituted C₁₋₄ alkyl.

34. The compound of claim 33, wherein R^{9A} is an optionally substituted C₁₋₄-alkyl.

35. The compound of claim 27, wherein 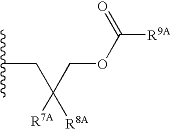
are each independently

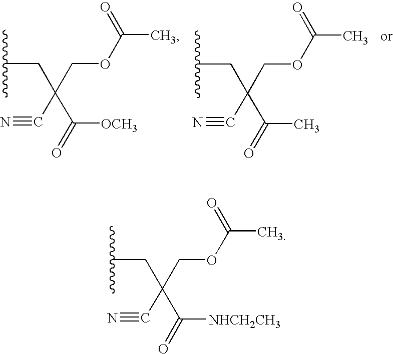

36. The compound of claim 27, wherein R^{5A} and R^{6A} are —C(═O)R^{10A}.

37. The compound of claim 36, wherein R^{10A} is unsubstituted or substituted C₁₋₄-alkyl.

38. The compound of claim 27, wherein R^{5A} and R^{6A} are —C(R^{11A})₂—O—C(═O)R^{12A}.

39. The compound of claim 38, wherein each R^{11A} is hydrogen and R^{12A} is unsubstituted or substituted C₁₋₄-alkyl.

40. The compound of claim 39, wherein R^{12A} is methyl or tert-butyl.

41. The compound of claim 27, wherein the compound of Formula (Ia) is selected from the group consisting of: 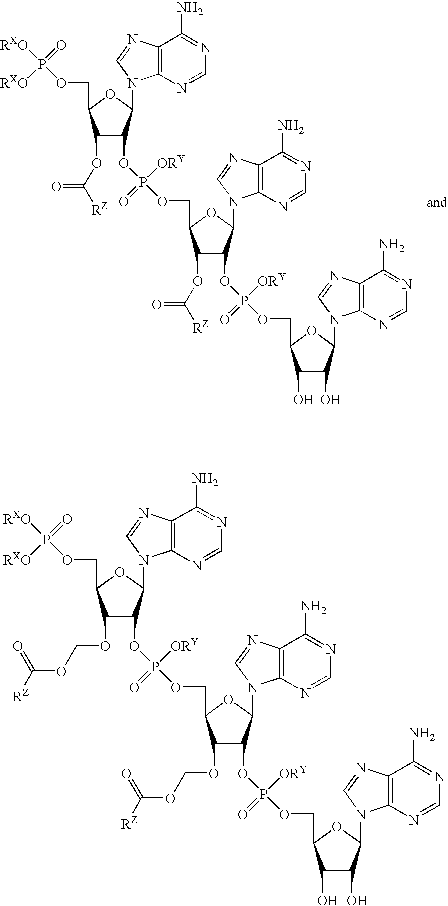
wherein: each R^{X} and each R^{Y} is 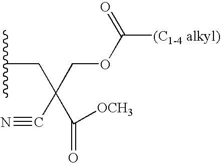
and each R² is selected from the group consisting of methyl, n-butyl and t-butyl.

42. A pharmaceutical composition comprising a compound of claim 1, and a pharmaceutically acceptable carrier, diluent, excipient or combination thereof.

43. A method of ameliorating or treating a neoplastic disease comprising administering to a subject suffering from a neoplastic disease a therapeutically effective amount of a compound of claim 1.

44. The method of claim 43, wherein the neoplastic disease is cancer.

45. The method of claim 43, wherein the neoplastic disease is a tumor.

46. The method of claim 45, wherein the tumor is a solid tumor.

47. The method of claim 43, wherein the neoplastic disease is leukemia.

48. The method of claim 47, wherein the leukemia is selected from the group consisting of acute lymphoblastic leukemia (ALL), acute myeloid leukemia (AML) and juvenile myelomonocytic leukemia (JMML).

49. A method of inhibiting the growth of a tumor comprising administering to a subject having the tumor a therapeutically effective amount of a compound of claim 1.

50. A method of ameliorating or treating a viral infection comprising administering to a subject suffering from a viral infection a therapeutically effective amount of a compound of claim 1.

51. The method of claim 50, wherein the viral infection is caused by a virus selected from the group consisting of an adenovirus, an Alphaviridae, an Arbovirus, an Astrovirus, a Bunyaviridae, a Coronaviridae, a Filoviridae, a Flaviviridae, a Hepadnaviridae, a Herpesviridae, an Alphaherpesvirinae, a Betaherpesvirinae, a Gammaherpesvirinae, a Norwalk Virus, an Astroviridae, a Caliciviridae, an Orthomyxoviridae, a Paramyxoviridae, a Paramyxoviruses, a Rubulavirus, a Morbillivirus, a Papovaviridae, a Parvoviridae, a Picornaviridae, an Aphthoviridae, a Cardioviridae, an Enteroviridae, a Coxsackie virus, a Polio Virus, a Rhinoviridae, a Phycodnaviridae, a Poxviridae, a Reoviridae, a Rotavirus, a Retroviridae, an A-Type Retrovirus, an Immunodeficiency Virus, a Leukemia Viruses, an Avian Sarcoma Viruses, a Rhabdoviruses, a Rubiviridae and a Togaviridae.

52. A method of ameliorating or treating a parasitic disease comprising administering to a subject suffering from a parasitic disease a therapeutically effective amount of a compound of claim 1.

53. The method of claim 52, wherein the parasitic disease is Chagas' disease.

54. A method of synthesizing a compound of Formula (I) comprising: 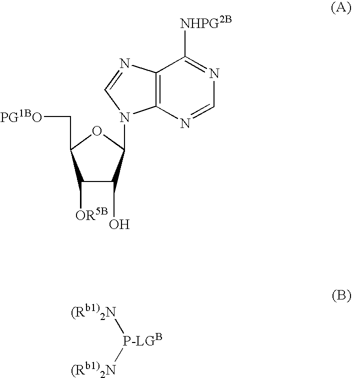
(a) forming phosphoamidite at the 2′-position of a compound of Formula A by reacting a compound of Formula B with the 2′-OH of the compound of Formula A to form a compound of Formula C; 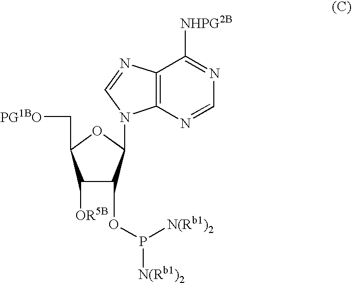
(b) adding R^{4B} to the compound of Formula C by reacting the compound of Formula C with a compound of Formula D to form a compound of Formula E: 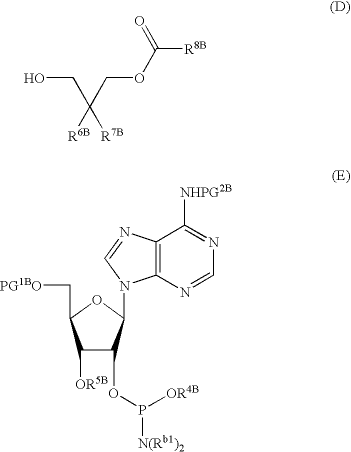
(c) adding NS^{2B}, wherein NS^{2B} has the structure of a compound of Formula F, to the compound of Formula E to form a compound of Formula G: 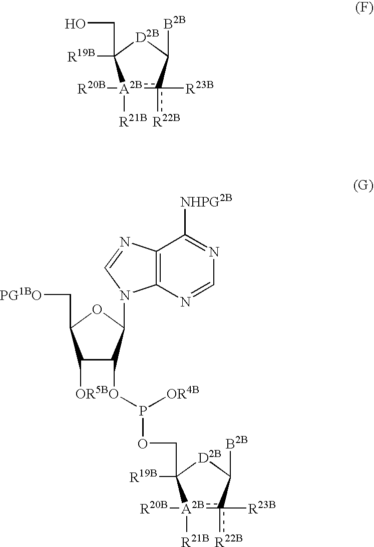
(d) oxidizing the phosphite of the compound of Formula G to a phosphate and forming a compound of Formula H; 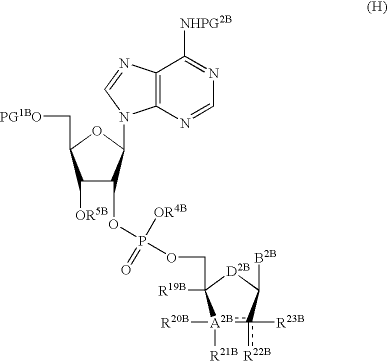
(e) removing PG^{1B} on the compound of Formula H to form a compound of Formula J: 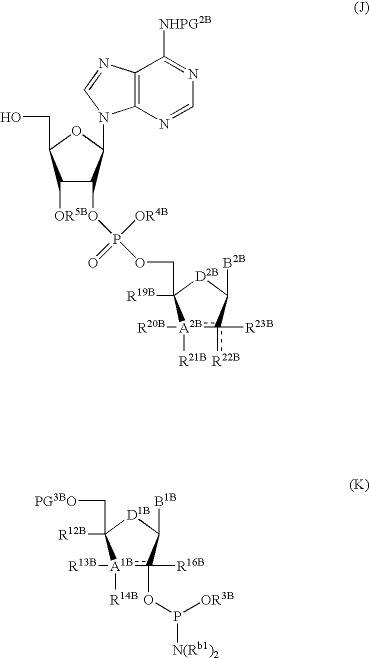
(f) adding NS^{1B}, wherein NS^{1B} has the structure of a compound of Formula K, to the 5′-OH of the compound of Formula J to form a compound of Formula L: 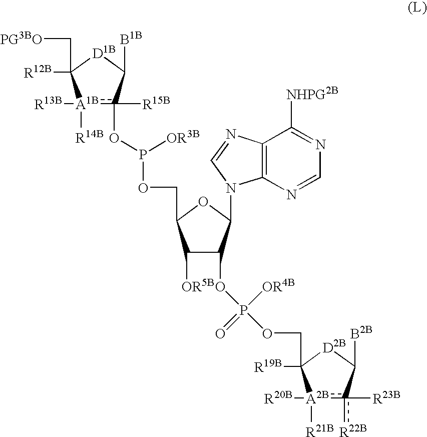
(g) oxidizing the phosphite of the compound of Formula L to a phosphate and forming a compound of Formula M; 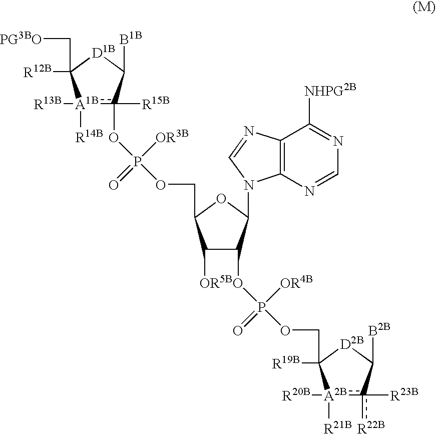
(h) removing PG^{3B} from the compound of Formula M to form a compound of Formula N: 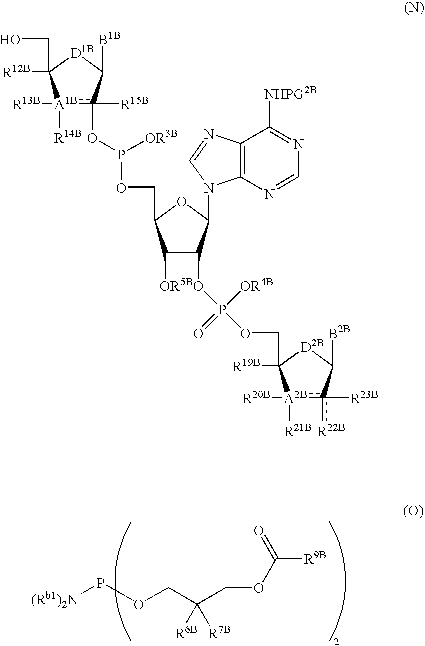
(i) adding a compound of Formula O to the 5′-OH on the compound of Formula N; and removing PG^{2B}, any protecting groups attached to the heterocyclic bases or the heterocyclic base derivatives of NS^{1B} and NS^{2B}, and any protecting group on to oxygens attached to NS^{1B} and NS^{2B} to form the compound of Formula (I);
wherein:
R^{1B}, R^{2B}, R^{3B} and R^{4B} are 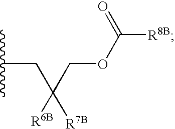
each R^{5B} are each independently selected from the group consisting of hydrogen, —C(═O)R^{9B}, and —C(R^{10B})₂—O—C(═O)R^{11B};
each R^{6B} and each R^{7B} are each independently selected from the group consisting of —C≡N, an optionally substituted 1-oxoalkyl, an optionally substituted alkoxycarbonyl and an optionally substituted alkylaminocarbonyl;
each R^{8B}, each R^{9B}, each R^{10B} and each R^{11B} are each hydrogen or an optionally substituted C₁₋₄-alkyl;
A^{1B} and A^{2B} are each independently selected from the group consisting of C, O and S;
D^{1B} and D^{2B} are each independently C═CH₂ or O;
B^{1B} and B^{2B} are each independently selected from the group consisting of an optionally substituted heterocyclic base, an optionally substituted heterocyclic base derivative, an optionally substituted protected heterocyclic base, and an optionally substituted protected heterocyclic base derivative;
R^{12B} is selected from the group consisting of hydrogen, azido, —CN, an optionally substituted C₁₋₄ alkyl and an optionally substituted C₁₋₄ alkoxy;
R^{13B} is absent or selected from the group consisting of hydrogen, halogen, hydroxy and an optionally substituted C₁₋₄ alkyl;
R^{14B} is absent or selected from the group consisting of hydrogen, halogen, azido, amino, hydroxy, —OC(═O)R^{16B}, and —OC(R^{17B})₂—O—C(═O)R^{18B};
R^{15B} is selected from the group consisting of hydrogen, halogen, hydroxy, —CN, —NC, an optionally substituted C₁₋₄ alkyl, an optionally substituted haloalkyl and an optionally substituted hydroxyalkyl;
each R^{16B}, each R^{17B} and each R^{18B} are independently hydrogen or an optionally substituted C₁₋₄-alkyl;
R^{19B} is selected from the group consisting of hydrogen, azido, —CN, an optionally substituted C₁₋₄ alkyl and an optionally substituted C₁₋₄ alkoxy;
R^{20B} is absent or selected from the group consisting of hydrogen, halogen, hydroxy and an optionally substituted C₁₋₄ alkyl;
R^{21B} is absent or selected from the group consisting of hydrogen, halogen, azido, amino, hydroxy and —OPG^{4B};
R^{22B} is selected from the group consisting of hydrogen, halogen, hydroxy, —CN, —NC, an optionally substituted C₁₋₄ alkyl, an optionally substituted C₁₋₄ alkoxy and —OPG^{5B};
R^{23B} is selected from the group consisting of hydrogen, halogen, hydroxy, —CN, —NC, an optionally substituted C₁₋₄ alkyl, an optionally substituted haloalkyl and an optionally substituted hydroxyalkyl, or when the bond to R^{22B} indicated by is a double bond, then R^{22B} and R^{23B} can be taken together to form a C₁₋₄ alkenyl;
each R^{b1} is independently an optionally substituted C₁₋₄ alkyl;
PG^{1B,} PG^{2B}, PG^{3B}, PG^{4B} and PG^{5B} are each independently a protecting group; and
LG^{B} is a leaving group.

55. The method of claim 54, wherein PG^{1B} and PG^{3B} are each a silyl ether protecting group.

56. The method of claim 54, wherein PG^{2B} is a triarylmethyl protecting group.

57. The method of claim 54, wherein PG^{4B} and PG^{5B} are each a levulinoyl group.

58. The method of claim 54, wherein B^{1B} and B^{2B} are each independently selected from: 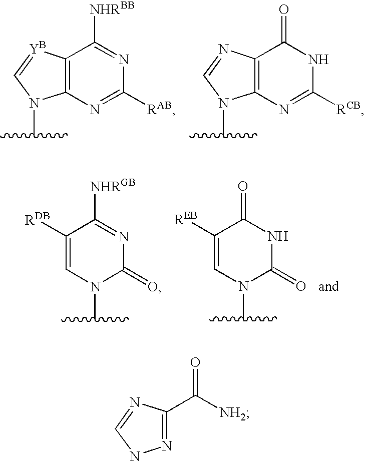
wherein:
R^{AB} is hydrogen or halogen;
R^{BB} is hydrogen, an optionally substituted C₁₋₄ alkyl, an optionally substituted C₃₋₈ cycloalkyl or a protecting group;
R^{CB} is hydrogen or amino;
R^{DB} is hydrogen or halogen;
R^{EB} is hydrogen or an optionally substituted C₁₋₄ alkyl;
Y^{B} can be N (nitrogen) or CR^{FB}, wherein R^{FB} hydrogen, halogen or an optionally substituted C₁₋₄ alkyl; and
R^{GB} can be a protecting group.

59. The method of claim 58 wherein R^{BB} and R^{GB} are triarylmethyl protecting groups.

60. A method of synthesizing a compound of Formula (Ia) comprising: 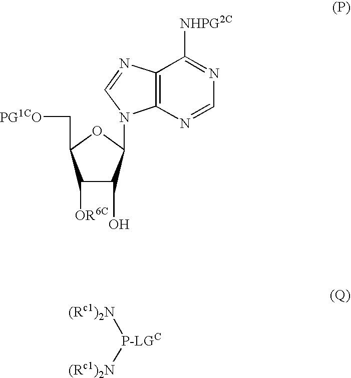
(a) forming phosphoamidite at the 2′-position of a compound of Formula P by reacting a compound of Formula Q with the 2′-OH of the compound of Formula P to form a compound of Formula R; 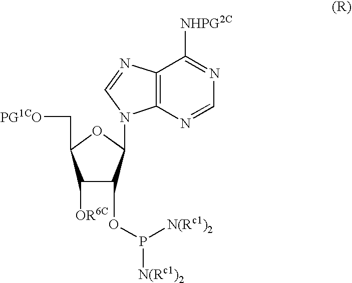
(b) adding R^{4C} to the compound of Formula R by reacting the compound of Formula R with a compound of Formula S to form a compound of Formula T: 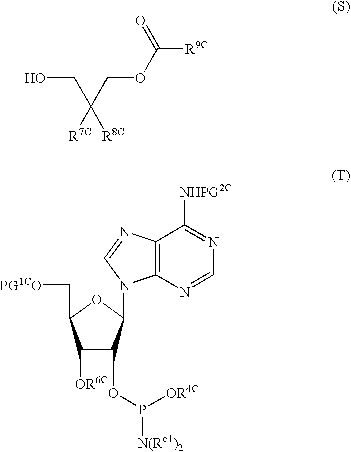
(c) adding a compound of Formula U to the compound of Formula T to form a compound of Formula V: 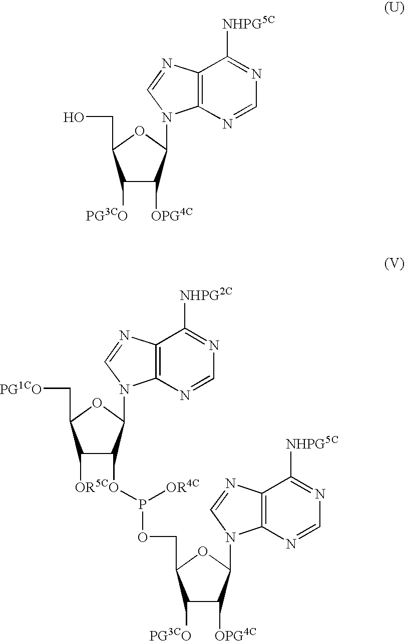
(d) oxidizing the phosphite of the compound of Formula V to a form a phosphate on a compound of Formula W; 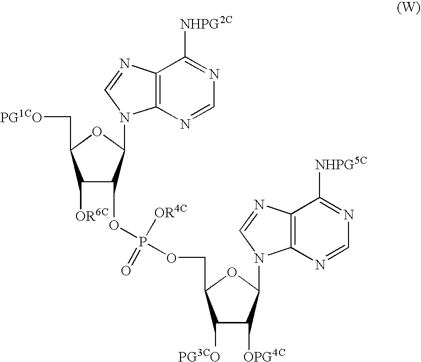
(e) removing PG^{1C} from the compound of Formula W to form a compound of Formula X: 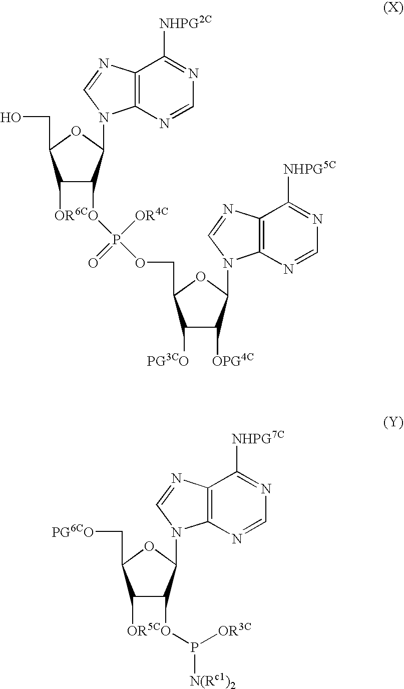
(f) adding a compound of Formula Y to the compound of Formula X to form a compound of Formula Z: 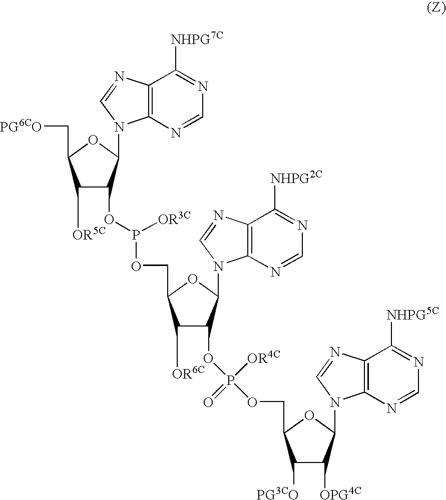
(g) oxidizing the phosphite of the compound of Formula Z to a form a phosphate and forming a compound of Formula AA; 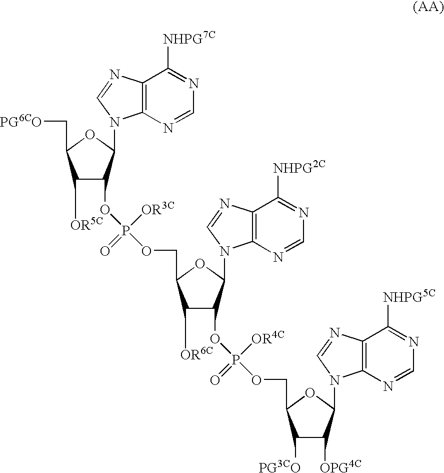
(h) removing PG^{6C} on the compound of Formula AA to form a compound of Formula BB: 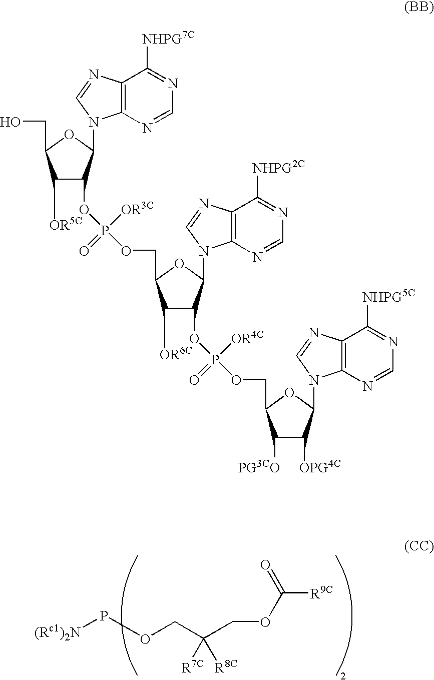
(i) adding a compound of Formula CC to the 5′-OH on the compound of Formula BB; and removing PG^{2C}, PG^{3C}, PG^{4C}, PG^{5C} and PG^{7C} to form a compound of Formula (Ia);
wherein:
R^{1C}, R^{2C}, R^{3C} and R^{4C} are each 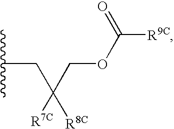
wherein R^{1C}, R^{2C}, R^{3C} and R^{4C} can be the same or different from each other;
R^{5C} and R^{6C} are independently selected from the group consisting of hydrogen, —C(═O)R^{10C}, and —C(R^{11C})₂—O—C(═O)R^{12C};
each R^{7C} and each R^{8C} are each independently selected from the group consisting of —C≡N, an optionally substituted 1-oxoalkyl, an optionally substituted alkoxycarbonyl and an optionally substituted alkylaminocarbonyl;
each R^{9C}, each R^{10C}, each R^{11C} and each R^{12C} are each hydrogen or an optionally substituted C₁₋₄-alkyl;
each R^{c1} is independently an optionally substituted C₁₋₄ alkyl;
PG^{1C}, PG^{2C}, PG^{3C}, PG^{4C}, PG^{5C}, PG^{6C} and PG^{7C} are each independently a protecting group; and
LG^{C} is a leaving group.

61. The method of claim 60, wherein PG^{1C} and PG^{6C} are each a silyl ether protecting group.

62. The method of claim 60, wherein PG^{2C}, PG^{5C} and PG^{7C} are each a triarylmethyl protecting group.

63. The method of claim 60, wherein PG^{3C} and PG^{4C} are each a levulinoyl group.
